SEQUENCE LISTING (9 sequences)

SEQ ID NO: 1 - protein, 291 aa, Homo sapiens
MRIFAVFIFMTYWHLLNAFTVTVPKDLYVVEYGSNMTIECKFPVEKQLDLAALIVYWEMEDKNIIQFVHGEEDLKVQHSSYRQRARLLKDQLSLGNAALQITDVKLPQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTHLVILGAILLCLGVALTFIFRLRKGRMMDVKKCGIQDTNSKKQSDTHLEET

SEQ ID NO: 2 - DNA, 1553 bp, Homo sapiens
  source 1..1553: mol_type = genomic DNA
cgaggctccgcaccagccgcgcttctgtccgcctgcagggcattccagaaagatgaggatatttgctgtctttatattcatgacctactggcatttgctgaacgcatttactgtcacggttcccaaggacctatatgtggtagagtatggtagcaatatgacaattgaatgcaaattcccagtagaaaaacaattagacctggctgcactaattgtctattgggaaatggaggataagaacattattcaatttgtgcatggagaggaagacctgaaggttcagcatagtagctacagacagagggcccggctgttgaaggaccagctctccctgggaaatgctgcacttcagatcacagatgtgaaattgcaggatgcaggggtgtaccgctgcatgatcagctatggtggtgccgactacaagcgaattactgtgaaagtcaatgccccatacaacaaaatcaaccaaagaattttggttgtggatccagtcacctctgaacatgaactgacatgtcaggctgagggctaccccaaggccgaagtcatctggacaagcagtgaccatcaagtcctgagtggtaagaccaccaccaccaattccaagagagaggagaagcttttcaatgtgaccagcacactgagaatcaacacaacaactaatgagattttctactgcacttttaggagattagatcctgaggaaaaccatacagctgaattggtcatcccagaactacctctggcacatcctccaaatgaaaggactcacttggtaattctgggagccatcttattatgccttggtgtagcactgacattcatcttccgtttaagaaaagggagaatgatggatgtgaaaaaatgtggcatccaagatacaaactcaaagaagcaaagtgatacacatttggaggagacgtaatccagcattggaacttctgatcttcaagcagggattctcaacctgtggtttaggggttcatcggggctgagcgtgacaagaggaaggaatgggcccgtgggatgcaggcaatgtgggacttaaaaggcccaagcactgaaaatggaacctggcgaaagcagaggaggagaatgaagaaagatggagtcaaacagggagcctggagggagaccttgatactttcaaatgcctgaggggctcatcgacgcctgtgacagggagaaaggatacttctgaacaaggagcctccaagcaaatcatccattgctcatcctaggaagacgggttgagaatccctaatttgagggtcagttcctgcagaagtgccctttgcctccactcaatgcctcaatttgttttctgcatgactgagagtctcagtgttggaacgggacagtatttatgtatgagtttttcctatttattttgagtctgtgaggtcttcttgtcatgtgagtgtggttgtgaatgatttcttttgaagatatattgtagtagatgttacaattttgtcgccaaactaaacttgctgcttaatgatttgctcacatctagtaaaacatggagtatttgtaaaaaaaaaaaaaaa

SEQ ID NO: 3 - protein, 9 aa, synthetic construct
LPYLGWLVF

SEQ ID NO: 4 - DNA, 22 bp, synthetic construct
  source 1..22: mol_type = other DNA
gctccaggatttctcggaagtc

SEQ ID NO: 5 - DNA, 22 bp, synthetic construct
  source 1..22: mol_type = other DNA
atggctactgctgtcaggtcca

SEQ ID NO: 6 - DNA, 22 bp, synthetic construct
  source 1..22: mol_type = other DNA
gttcaggacagcgacttctgga

SEQ ID NO: 7 - DNA, 22 bp, synthetic construct
  source 1..22: mol_type = other DNA
gaaggagaactccgctgactct

SEQ ID NO: 8 - DNA, 22 bp, synthetic construct
  source 1..22: mol_type = other DNA
actaccaagccagtgctgcgaa

SEQ ID NO: 9 - DNA, 22 bp, synthetic construct
  source 1..22: mol_type = other DNA
atcacaggcgaagtccaatccg

(19) United States
(12) Patent Application Publication
(10) Pub. No.: US 2025/0235403 A1
(43) Pub. Date: Jul. 24, 2025

(54) COMPOSITIONS AND METHODS OF THE DELIVERY OF ACTIVE AGENTS INCLUDING NUCLEIC ACIDS

(71) Applicant: Ohio State Innovation Foundation, Columbus, OH (US)

(72) Inventors: Chun-Tien KUO, New Rochelle, NY (US); Zhongkun ZHANG, Columbus, OH (US); Robert J. LEE, Lewis Center, OH (US)

(21) Appl. No.: 18/703,633

(22) PCT Filed: Oct. 24, 2022
(86) PCT No.: PCT/US2022/078612

§ 371 Date: Apr. 22, 2024

Related U.S. Application Data
(60) Provisional application No. 63/288,152, filed on Dec. 10, 2021.
(60) Provisional application No. 63/270,724, filed on Oct. 22, 2021.
(60) Provisional application No. 63/270,719, filed on Oct. 22, 2021.

(51) Int. Cl. A61K 9/1271; A61K 47/69; A61K 48/00; C12N 15/88

(52) U.S. Cl. CPC A61K 9/1271; A61K 47/6927; A61K 48/0008; A61K 48/005; C12N 15/88

(57) ABSTRACT

Disclosed herein are pH-sensitive nanoemulsions as well as methods of using thereof. These pH-sensitive nanoemulsions can comprise a lipid particle encapsulating an active agent. The lipid particle can comprise one or more ionizable lipids; one or more neutral lipids; one or more PEGylated lipids; and optionally one or more fusogenic oils. In some embodiments, these compositions can be buffered at an acidic pH (e.g., a pH of less than 6.5, such as a pH of from 4 to 6.5, or a pH of from 5.0 to 6.5). By buffering at an acidic pH, the delivery efficiency of the compositions can be enhanced as compared to otherwise identical compositions buffered at a pH of 7 or more.

### CROSS-REFERENCE TO RELATED APPLICATIONS

This application claims benefit of U.S. Provisional Application No. 63/270,719, filed Oct. 22, 2021, U.S. Provisional Application No. 63/270,724, filed Oct. 22, 2021, and U.S. Provisional Application No. 63/288,152, filed Dec. 10, 2021, each of which is hereby incorporated herein by reference in its entirety.

### BACKGROUND

Therapeutic nucleic acids include, e.g., small interfering RNA (siRNA), micro RNA (miRNA), antisense oligonucleotides, ribozymes, plasmids, mRNA, sgRNA, and immune stimulating nucleic acids. These nucleic acids act via a variety of mechanisms. In the case of siRNA or miRNA, these nucleic acids can down-regulate intracellular levels of specific proteins through a process termed RNA interference (RNAi). Following introduction of siRNA or miRNA into the cell cytoplasm, these double-stranded RNA constructs can enter into a protein complex termed RISC. The sense strand of the siRNA or miRNA is displaced from the RISC complex providing a template within RISC that can recognize and bind mRNA with a complementary sequence to that of the bound siRNA or miRNA. Having bound the complementary mRNA the RISC complex cleaves the mRNA and releases the cleaved strands. RNAi can provide down-regulation of specific proteins by targeting specific destruction of the corresponding mRNA that encodes for protein synthesis.

The therapeutic applications of RNAi are extremely broad, since siRNA and miRNA constructs can be synthesized with any nucleotide sequence directed against a target protein. To date, siRNA constructs have shown the ability to specifically down-regulate target proteins in both in vitro and in vivo models. In addition, siRNA constructs are currently being evaluated in clinical studies.

In spite of recent progress, there remains a need in the art for improved lipid-therapeutic nucleic acid compositions that are suitable for general therapeutic use. These compositions would, for example, encapsulate nucleic acids with high-efficiency, have high drug: lipid ratios, protect the encapsulated nucleic acid from degradation and clearance in serum, be suitable for systemic delivery, and provide intracellular delivery of the encapsulated nucleic acid. In addition, these lipid-nucleic acid particles should be well-tolerated and provide an adequate therapeutic index, such that patient treatment at an effective dose of the nucleic acid is not associated with significant toxicity and/or risk to the patient

### SUMMARY

Provided herein are pharmaceutical compositions that comprise a lipid particle encapsulating an active agent. The lipid particle can comprise one or more ionizable lipids; one or more neutral lipids; and one or more PEGylated lipids. These compositions can be buffered at an acidic pH (e.g., a pH of less than 6.5, such as a pH of from 4 to 6.5, or a pH of from 5.0 to 6.5). By buffering at an acidic pH, the delivery efficiency of the compositions can be enhanced as compared to otherwise identical compositions buffered at a pH of 7 or more.

In some embodiments, the one or more ionizable lipids are present in the lipid particle in an amount of from 20 mol % to 65 mol % (e.g., from 30 mol % to 50 mol %) of the total components forming the lipid particle. In some embodiments, the one or more ionizable lipids can comprise a lipid headgroup comprising a tertiary amine. In certain embodiments, the one or more ionizable lipids can comprise N,N-dimethyl-2,3-dioleyloxypropylamine (DODMA), [(4-hydroxybutyl)azanediyl]di(hexane-6,1-diyl)bis(2-hexyldecanoate) (ALC-0315); 9-heptadecanyl 8-{(2-hydroxyethyl) [6-oxo-6-(undecyloxy) hexyl]amino}octanoate (SM-102), DLin-MC3-DMA; DLin-KC2-DMA; or any combination thereof.

In some embodiments, the one or more neutral lipids are present in the lipid particle in an amount of from 35 mol % to 80 mol % (30 mol % to 50 mol %) of the total components forming the lipid particle. The one or more neutral lipids can comprise any suitable neutral lipid. For example, in some embodiments, the one or more neutral lipids can comprise dipalmitoylphosphatidylcholine (DPPC), dioleoylphosphatidylethanolamine (DOPE), palmitoyloleoylphosphatidylcholine (POPC), egg phosphatidylcholine (EPC), distearoylphosphatidylcholine (DSPC), cholesterol, or any combination thereof.

In some embodiments, the one or more PEGylated lipids are present in the lipid particle in an amount of from greater than 0 mol % to 5 mol % of the total components forming the lipid particle. The one or more PEGylated lipids can comprise, for example, a PEG-ditetradecylacetamide, a PEG-myristoyl diglyceride, a PEG-diacylglycerol, a PEG dialkyloxypropyl, a PEG-phospholipid, a PEG-ceramide, or any combinations thereof.

The lipid particles can have an average diameter of less than 1 micron, such as from from 50 nm to 750 nm, 50 nm to 250 nm, from 50 nm to 200 nm, from 50 nm to 150 nm, or from 50 nm to 100 nm. The lipid particles can have a polydispersity index (PDI) of less than 0.4.

The active agent encapsulated in the lipid particles can comprise any suitable active agent, such as a small molecule therapeutic agent, a diagnostic agent, a peptide, a protein, an antibody, or a nucleic acid. In certain embodiments, the active agent can comprise a nucleic acid, such as siRNA, mRNA, or any combination thereof.

Also provided are pharmaceutical compositions that comprise a lipid particle encapsulating an active agent, the lipid particle comprising: from 20 mol % to 65 mol % one or more ionizable lipids; from 35 mol % to 80 mol % one or more neutral lipids; from greater than 0 mol % to 5 mol % one or more PEGylated lipids; and from 5 mol % to 50 mol % one or more fusogenic oils. By incorporating the one or more fusogenic oils, the delivery efficiency of the compositions can be enhanced as compared to otherwise identical compositions lacking the one or more fusogenic oils.

Optionally, these compositions can be buffered at an acidic pH (e.g., a pH of less than 6.5, such as a pH of from 4 to 6.5, or a pH of from 5.0 to 6.5). By buffering at an acidic pH, the delivery efficiency of the compositions can be enhanced as compared to otherwise identical compositions buffered at a pH of 7 or more.

The one or more fusogenic oils can be present in the lipid particle in an amount of from 10 mol % to 40 mol % of the total components forming the lipid particle. In some embodiments, the fusagenic oil can comprise a C12-C40 hydrocarbon comprising fewer than 3 rings. In some cases, the C12-C40 hydrocarbon can comprise an alkyl or alkylene chain, such as an alkylene chain optionally comprises a least one cis-double bond. For example, the fusogenic oil can comprise squalene, squalane, pristane, pristene, farnesene, farnesane, retinol, phytol, a carotene, a tocopherol, a tocotrienol, phytomenadione, menaquinone, where valence permits esters thereof, or a combination thereof. In certain embodiments, the fusogenic oil can comprise squalene.

In some embodiments, the one or more ionizable lipids are present in the lipid particle in an amount of from 30 mol % to 50 mol % of the total components forming the lipid particle. The fusogenic oil and the one or more ionizable lipids can be present in the lipid particles at a molar ratio of from 0.25:1 to 1:1. In some embodiments, the one or more ionizable lipids can comprise a lipid headgroup comprising a tertiary amine. In certain embodiments, the one or more ionizable lipids can comprise N,N-dimethyl-2,3-dioleyloxypropylamine (DODMA), [(4-hydroxybutyl)azanediyl]di(hexane-6,1-diyl)bis(2-hexyldecanoate) (ALC-0315); 9-heptadecanyl 8-{(2-hydroxyethyl) [6-oxo-6-(undecyloxy) hexyl]amino}octanoate (SM-102), DLin-MC3-DMA; DLin-KC2-DMA; or any combination thereof.

In some embodiments, the one or more neutral lipids are present in the lipid particle in an amount of from 35 mol % to 80 mol % (30 mol % to 50 mol %) of the total components forming the lipid particle. The one or more neutral lipids can comprise any suitable neutral lipid. For example, in some embodiments, the one or more neutral lipids can comprise dipalmitoylphosphatidylcholine (DPPC), dioleoylphosphatidylethanolamine (DOPE), palmitoyloleoylphosphatidylcholine (POPC), egg phosphatidylcholine (EPC), distearoylphosphatidylcholine (DSPC), cholesterol, or any combination thereof.

In some embodiments, the one or more PEGylated lipids are present in the lipid particle in an amount of from greater than 0 mol % to 5 mol % of the total components forming the lipid particle. The fusogenic oil and the one or more PEGylated lipids can be present in the lipid particles at a molar ratio of from 5:1 to 20:1. The one or more PEGylated lipids can comprise, for example, a PEG-ditetradecylacetamide, a PEG-myristoyl diglyceride, a PEG-diacylglycerol, a PEG dialkyloxypropyl, a PEG-phospholipid, a PEG-ceramide, or any combinations thereof.

The lipid particles can have an average diameter of less than 1 micron, such as from from 50 nm to 750 nm, 50 nm to 250 nm, from 50 nm to 200 nm, from 50 nm to 150 nm, or from 50 nm to 100 nm. The lipid particles can have a polydispersity index (PDI) of less than 0.4.

The active agent encapsulated in the lipid particles can comprise any suitable active agent, such as a small molecule therapeutic agent, a diagnostic agent, a peptide, a protein, an antibody, or a nucleic acid. In certain embodiments, the active agent can comprise a nucleic acid, such as siRNA, mRNA, or any combination thereof.

The compositions described herein can be used to deliver one or more active agents to cells (e.g., in vivo, ex vivo, or in vitro). Accordingly, provided herein are method of delivering an active agent to a cell (e.g., in vivo, ex vivo, or in vitro) that comprise contacting the cell with a composition described herein. Also provided are methods for in vivo delivery of an active agent to a cell, said method comprising administering to a mammalian subject (e.g., a human) a composition described herein. In some embodiments, the administration can comprise systemic administration (eg., intravenous injection or infusion).

### DESCRIPTION OF DRAWINGS

FIG. 1 schematically illustrates the structure of R848-loaded squalene emulsion and chemical components.

FIGS. 2A-2C illustrate particle characterizations of R848 nanoemlusions. FIG. 2A shows the particle sizes of R848 NEs with lipid-to-R848 weight ratio of 20:1, 15:1, 10:1, 5:1, and 2:1. FIG. 2B shows the SEC chromatogram of R848 NE using a Sepharose CL-4B gel column. Absorbance at 320 nm was measured for the presence of R848. FIG. 2C shows the particle stability of empty NE and R848 NE stored at 4° C., up to 3 weeks

FIG. 3 shows 200× brightfield images of RAW 264.7 cells after 12 hours treated with (Panel A) complete medium only, (Panel B) empty NE, (Panel C) free R848, (Panel D) SD-101, (Panel E) R848 NE, and (Panel F) R848 NE with SD-101. R848 was treated at 50 μM individually, in emulsion, or in combination. SD-101 was treated at 300 nM individually or in combination.

FIGS. 4A-4C show the TNF-α (FIG. 4A), IL-6 (FIG. 4B), and IL-12p70 (FIG. 4C) concentrations secreted by RAW 264.7 cells after 12 hours treated with complete medium-only, empty NE, free R848, free SD-101, R848 NE, or R848 NE/SD-101 combination. R848 was treated at 50 μM individually, in nanoemulsion, or in combination. SD-101 was treated at 300 nM individually or in combination. One-way ANOVA: * p<0.05, **: p<0.01, *** p<0.001

FIG. 5 illustrates R848 NE and SD-101 treatments of murine colon adenocarcinoma (MC38) syngeneic C57BL/6N mouse model. Panel A shows a timeline for MC38 inoculation and treatment regimen. The mice were inoculated with 0.5 million MC38 subcutaneously on the right flank. Treatments began at 8 days after inoculation when tumors became palpable. Treatments were given every 3 days for up to 4 doses. Mice were euthanized after the fourth dose at day 10. Panels B-E show tumor growth over time for individual mice treated with (Panel B) saline, (Panel C) R848 NE, (Panel D) SD-101, or (Panel E) R848 NE/SD-101 combination (n=5). Panel F shows images of MC38 tumor tissues collected at day 10 with measured (Panel G) tumor sizes. Panel His a plot of the average tumor growth for each treatment group within 10 days. Data are presented as means±SEM (n=5). One-way ANOVA: * p<0.05, **: p<0.01, *** p<0.001.

FIG. 6A shows spleen tissues collected from mice at day 10. FIG. 6B is a plot of the measured spleen weights, normalized to individual body weight (n=5). One-way ANOVA* p<0.05, **: p<0.01, *** p<0.001.

FIGS. 7A-7C show the TNF-α (FIG. 7A), IL-6 (FIG. 7B), and IL-12p70 (FIG. 7C) concentration in mouse serum. Serum samples were isolated from whole blood at day 10 (n=3). One-way ANOVA: * p<0.05, **: p<0.01, *** p<0.001.

FIG. 8A-8B show the gene regulation of (FIG. 8A) Akt1, Bcl2, Pdl1, Calreticulin, Hmgb1, Cd3e, Cd4, and Cd8a in tumor tissues collected from C57BL/6N mice, and (FIG. 8B) Akt1, Bcl2, Pdl1, Foxp3, Ifng in spleen tissues collected from the mice within the same study. Data are presented as means±SEM (n=3). One-way ANOVA: * p<0.05, **: p<0.01, *** p<0.001.

FIG. 9 includes plots showing the PSNE particle sizes (left) and polydispersity indexes (PDI, right).

FIGS. 10A-10B show the results of a gel retardation assay (FIG. 10A) examining oligonucleotide encapsulation and size exclusion chromatogram (FIG. 10B) examining Resiquimod encapsulation. In FIG. 10A, Ln: 1: SD-101 ODN, 2: anti-PDL1 LNA ASO, 3: empty PSNE LNP, 4: empty PSNE LNP with Resiquimod, 5: PSNE/SD-101 ODN, 6: PSNE/anti-PDL1 LNA ASO, 7: PSNE/(SD-101 ODN+anti-PDL1 LNA ASO), 8: PSNE/(SD-101 ODN+Resiquimod.

FIG. 11 shows tumor growth curves of MC-38 subcutaneous murine syngeneic models treated with various PSNE lipid nanoparticle constructs throughout the treatment period.

FIG. 12 shows a tumor weight analysis and tumor size comparison.

FIG. 13 shows a spleen index analysis and spleen size comparison.

FIG. 14 shows the results of a flow cytometry analysis on various immune cells of interest. Gating Strategies: CD8⁺ T lymphocytes: CD3⁺CD8⁺; CD4⁺ T lymphocytes: CD3⁺CD4⁺; Regulatory T lymphocytes: CD3⁺CD4⁺Foxp3⁺; MDSC: CD11b⁻CD11c⁺Gr-1⁺.

FIG. 15 shows the results of a flow cytometry analysis of surface PD-L1 expression on immune cells (Left: CD8⁺ T lymphocytes, CD3⁺CD8⁺; Right: Macrophages, CD11b⁺F4/80⁺).

FIG. 16 shows the results of an RT-qPCR analysis on murine PD-L1 mRNA level in spleen.

FIG. 17 shows the results of a cytokine analysis (IL-10, IL-12p70, TNFα, IFNγ) by enzyme-linked immunosorbent assay (ELISA).

FIGS. 18A and 18B show the amino acid (SEQ ID NO: 1) and nucleotide (SEQ ID NO: 2) sequences, respectively, of human PD-L1. The signal peptide is indicated in italics in the amino acid sequence, and the coding region is indicated in bold in the nucleotide sequence.

FIG. 19 is a plot showing the tumor progression profiles of mice treated with various TLR agonist-incorporated cationic nanoemulsions.

FIG. 20 shows the tumor progression profiles of mice treated with various TLR agonist-incorporated cationic nanoemulsions, individual groups. Panel A. Normal saline. Panel B. Squalene vehicle control. Panel C. poly (I: C) cationic nanoemulsions. Panel D. CpG 2216 cationic nanoemulsions. Panel E. Imiquimod squalene nanoemulsions.

FIG. 21 shows a J558 tumor challenge on mice immunized with different cancer vaccine constructs.

FIG. 22 shows tumor progression profiles of mice treated with SD-101 CpG ODN and/or 2′-OMe anti-PDL1 ASO in standard LNPs.

FIG. 23 shows body weight profiles of mice treated with SD-101 CpG ODN and/or 2′-OMe anti-PDL1 ASO in standard LNPs.

FIG. 24 shows a gene downregulation efficacy analysis of different chemical modifications of anti-murine PD-L1 ASO utilizing RT-qPCR.

FIG. 25 shows the MC-38 tumor progression profile of mice treated with PSNE-based lipid nanoparticles (trial 1).

FIG. 26 shows the particle size and polydispersity index analysis of different PSNE LNP constructs encapsulating SD-101, anti-PDL1 LNA ASO, and resiquimod.

FIG. 27 shows a gel retardation assay assessing oligonucleotide encapsulation efficiencies of different PSNE LNPs.

FIG. 28 shows a size exclusion chromatography elusion chromatogram of Resiquimod-encapsulated PSNE on a CL-4B SEC column.

FIG. 29 shows individual mouse tumor progression curves in different PSNE lipid nanoparticle treatment groups.

FIG. 30 shows the MC-38 tumor progression profile of mice treated with PSNE-based lipid nanoparticles (trial 2).

FIG. 31 shows the tumor weight analysis of each PSNE lipid nanoparticle treatment group, along with images of tumors.

FIG. 32 shows the spleen weight/index analysis of each PSNE lipid nanoparticle treatment group, along with images of tumors.

FIG. 33 shows a splenocyte population analysis of each PSNE lipid nanoparticle treatment group through flow cytometry.

FIG. 34 shows surface PD-L1 expression analysis on splenic cytotoxic T lymphocytes and macrophages through flow cytometry.

FIG. 35 shows the results of a splenocyte Pdl1 mRNA level analysis on each PSNE lipid nanoparticle treatment group through RT-qPCR

FIG. 36 shows splenocyte cytokine (Il10 and Il12-p40) mRNA level analysis of each PSNE lipid nanoparticle treatment group through RT-qPCR.

FIG. 37 shows serum cytokine level analysis on each PSNE lipid nanoparticle treatment group through cytokine ELISA.

FIG. 38 shows murine Pdl1 mRNA regulation utilizing anti-PDL1 LNA ASO on different next generation PSNE LNP constructs

FIG. 39 shows murine Pdl1 mRNA regulation on Hepa1-6 and MC-38 cells utilizing next generation PSNE-encapsulated anti-PDL1 LNA ASO along with IFN-γ induction

FIGS. 40A-40B shows murine PD-L1 surface protein expression on Hepa1-6 (FIG. 40A) and MC-38 (FIG. 40B) cells utilizing next generation PSNE-encapsulated anti-PDL1 LNA ASO along with IFN-γ induction using flow cytometry.

FIG. 41 shows surface protein expression on RAW264.7 cells utilizing next generation PSNE-encapsulated SD-101/anti-PDL1 LNA ASO along with LPS induction using flow cytometry analysis. The left panel shows CD86 MI macrophage activation marker, BV605. The right panel shows PD-L1, APC.

FIG. 42 shows MC-38 cytotoxic analysis utilizing RAW264.7 condition media.

FIG. 43 shows MC-38 tumor progression profile of mice treated with next gen PSNE-based lipid nanoparticles, whole/selected treatment peried.

FIG. 44 shows individual mouse tumor progression curves in different next gen PSNE lipid nanoparticle treatment groups. Panel A, Normal saline; Panel B, PSNE-Chol/M5-SD-101; Panel C, PSNE-Chol/M5-anti-PDL1 LNA ASO; Panel D, PSNE-Chol/M5 Mixture; and Panel E, PSNE-Chol/M5 co-loading.

FIG. 45 shows a body weight analysis of tumor-bearing mice, treated with next gen PSNE based lipid nanoparticles. Panel A, Normal saline; Panel B, PSNE-Chol/M5-SD-101; Panel C, PSNE-Chol/M5-anti-PDL1 LNA ASO; Panel D, PSNE-Chol/M5 Mixture; and Panel E, PSNE-Chol/M5 co-loading.

FIG. 46 shows the tumor weight (Panel A) and spleen index (Panel B) analysis of each next gen PSNE lipid nanoparticle treatment groups.

FIG. 47 shows a splenocyte population analysis of each next gen PSNE lipid nanoparticle treatment group through flow cytometry. Panel A, CD4+ T lymphocytes; and Panel B, CD8+ T lymphocytes (cytotoxic T lymphocytes).

FIG. 48 shows a splenic regulatory T lymphocyte population analysis of each next gen PSNE lipid nanoparticle treatment group through flow cytometry

FIG. 49 shows a splenic mRNA level analysis on each next gen PSNE lipid nanoparticle treatment group through RT-qPCR. Panel A, Pdl1; Panel B, Siglech, a marker for plasmacytoid dendritic cells; Panel C, Foxp3, a marker for regulatory T lymphocytes

FIG. 50 shows tumor Pdl1 mRNA level analysis on each next gen PSNE lipid nanoparticle treatment group through RT-qPCR

FIG. 51 shows the results of a splenocyte cytokine mRNA level analysis on each next gen PSNE lipid nanoparticle treatment group through RT-qPCR. Panel A, TNF-α; Panel B, IFN-Y, Panel C, IL-10; Panel D, IL-6; and Panel E, TGF-B.

FIG. 52 shows the results of a hepatic Pdl1 mRNA level analysis of anti-PDL1 LNA ASO delivery by next gen PSNE lipid nanoparticles through RT-qPCR.

FIGS. 53A-53C show the particle characterization of R848-IVM NE. FIG. 53A shows the particle sizes of empty NE, R848 NE, IVM NE, and R848-IVM NE. FIG. 53B shows a SEC chromatogram of R848-IVM NE using a Sepharose CL-4B column. Absorbance at 320 nm and 245 nm were used to measure the presence of R848 and IVM, respectively. FIG. 53C shows the solubility of R848 and IVM in squalene NE and PBS determined by HPLC.

FIGS. 54A-54B show cell viability and IC50 determination of R848 and IVM. FIG. 54A shows the results obtained from free R848 and R848 NE used to treat MC38 cells for 72 hours followed by an MTS assay. FIG. 54B shows the results obtained from free IVM and IVM NE used to treat MC38 cells for 72 hours followed by an MTS assay. Data were presented as means±SD (n=3).

FIGS. 55A-55B show gene regulation of Calreticulin, Hmgb1, and Lc3b in MC38 cells. Cells were treated with (FIG. 55A) free R848 and R848 NE, or (FIG. 55B) free IVM and IVM NE. R848 and IVM were treated at 8 μM respectively as free drugs or in squalene-based NE. Data are presented as means±SD (n=3). One-way ANOVA: * p<0.05, **: p<0.01, **, *** p<0.001.

FIGS. 56A-56D show MC38 migration in response to treatment with R848 and IVM. FIG. 56A shows cell morphology before and after being treated with 8 μM of R848 NE, IVM NE, and R848-IVM NE. FIG. 56B shows the percentage of MC38 cells migrated into the wound region evaluated 24 hours following the generation of a scratch wound across confluent cells. FIG. 56C shows that R848-IVM NE dose-dependent inhibition in MC38 migration was determined. FIG. 56D shows gene regulation in Calreticulin, Hmgb1, and Lc3b in MC38 migration study treated with R848 NE, IVM NE, and R848-IVM NE (n=3). One-way ANOVA: * p<0.05, ** p<0.01, *** p<0.001.

FIG. 57 shows R848 NE, IVM NE, and R848-IVM NE treatments on murine colon adenocarcinoma (MC38) syngeneic C57BL/6N mouse model. (Panel A) Graphic illustration of tumor inoculation and treatment regimen. Mice were inoculated with 1 million MC38 cells subcutaneously on the right flanks. Treatments began 7 days after tumor inoculation when tumor sizes reached 100 mm³. Treatments were given every 3 days for 3 doses. Mice were euthanized after the third dose on day 9. (Panel B-Panel E) Tumor growth over time for individual mice treated with (Panel B) saline, (Panel C) R848 NE, (Panel D) IVM NE, and (Panel E) R848-IVM NE (n=5). (Panel F) Images of MC38 tumor tissues were collected on day 10 with measured (Panel G) tumor weights. (Panel H) Average tumor growth for each treatment group within 9 days. Data are presented as means±SEM (n=5). One-way ANOVA: * p<0.05, ** p<0.01, *** p<0.001.

FIGS. 58A-58D show gene regulation and immune cell populations in in tumor and spleen tissues from treated mice. (FIG. 58A) Calreticulin, Hmgb1, Lc3b, Cd3e, Cd4, and Cd8a mRNA expressions in tumor tissues collected from C57BL/6N mice. (FIGS. 58B-58C) Percentage of CTLs (FIG. 58B) and the ratio of CTL to Tregs (FIG. 58C) were determined by flow cytometry in the spleen tissues. (FIG. 58D) The protein expression of HMGB1 and Ki67 was determined by western blot in the same tumor tissues at (FIG. 58A). Data are presented as means±SD (n=3). One-way ANOVA: * p<0.05, ** p<0.01, *** p<0.001.

FIGS. 59A-59B show the results of studies demonstrating the efficacy of squalene as a fusogenic agent in pH-sensitive nanoemulsions.

FIG. 60 is a plot showing the downregulation of the Akt-1 gene by PSNE-siRNA compositions formulated at pH 7.4, 6.5, and 5.5. The y-axis represents the relevant mRNA level following administration of the PSNE-siRNA composition. Akt-1 mRNA was measured by qRT-PCR. A negative control is included for reference.

FIGS. 61A-61E show IVIS in vivo bioluminescence imaging of mice treated with i.m. injection of FFLuc mRNA (naked mRNA), FFLuc mRNA encapsulated in LNPs, FFLuc mRNA encapsulated in pH-sensitive micelles (PSMs), and FFLuc mRNA encapsulated in PSNEs (buffered at pH 6). As shown in FIGS. 3A-3E, the PSM and PSNE formulations were far more active than the LNP formulation, with the PSNE formulation exhibiting the highest activity in mRNA delivery.

FIG. 62 shows the delivery afforded by PSNEs based on DLin-MC3-DMA and based on DODMA carrying luciferase mRNA given to mice by i.m. injection. A high level of gene expression were seen for PSNEs in 50 mM pH 6 histidine buffer but not for PSNEs in 50 mM phosphate buffer pH 7.4. This suggests a very strong pH dependence for the formulation for i.m. delivery of mRNA. The same principles are applicable to LNPs containing appropriate ionizable lipids.

FIG. 63 is a plot quantifying the increase in bioluminesnce observed following i.m. injection of PSNEs based on DLin-MC3-DMA carrying luciferase mRNA buffered in 50 mM pH 6 histidine buffer versus 50 mM phosphate buffer pH 7.4. A 30-50x enhancement in luminsence was observed by lowering the pH from 7.4 to 6.

FIG. 64 includes plots showing the cellular uptake of fluorescence-labeled PSNEs in KB cells at differing buffering pH. The results showed much higher levels of cellular uptake (increased mean fluorescence intensity) by flow cytometry analysis as pH S and pH 6 as compared to pH 7. This illustrates a mechanism of pH-dependent cellular uptake/delivery of PSNES.

FIG. 65 shows the design of a syringe assembly with integrated size-exclusion cartridge for in-line ethanol removal and buffer exchange.

FIGS. 66A-66C are photographs showing a 4-syringe+1-cartridge assembly for instant generation of ethanol-free nanoparticles. The yield (% recovery of nucleic acid compared to input) for producing ethanol-free nanoparticles using this device was greater than 60% (specifically 68.8%).

### DETAILED DESCRIPTION

### Definitions

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

“Aqueous solution” refers to a composition comprising in whole, or in part, water.

“Organic lipid solution” refers to a composition comprising in whole, or in part, an organic solvent having a lipid. In some embodiments, the organic lipid solution can comprise an alkanol, most preferably ethanol. In certain embodiments, the compositions described herein can be free of organic solvents, such as ethanol.

“Lipid” refers to a group of organic compounds that are esters of fatty acids and are characterized by being insoluble in water but soluble in many organic solvents, e.g, fats, oils, waxes, phospholipids, glycolipids, and steroids.

“Amphipathic lipid” comprises a lipid in which hydrophilic characteristics derive from the presence of polar or charged groups such as carbohydrates, phosphato, carboxylic, sulfato, amino, sulfhydryl, nitro, hydroxy and other like groups, and hydrophobic characteristics can be conferred by the inclusion of a polar groups that include, but are not limited to, long chain saturated and unsaturated aliphatic hydrocarbon groups and such groups substituted by one or more aromatic, cycloaliphatic or heterocyclic group(s). Examples include phospholipids, aminolipids and sphingolipids. Phospholipids include phosphatidylcholine, phosphatidylethanolamine, phosphatidylserine, phosphatidylinositol, phosphatidic acid, palmitoyloleoyl phosphatidylcholine, lysophosphatidylcholine, lysophosphatidylethanolamine, dipalmitoylphosphatidylcholine, dioleoylphosphatidylcholine, distearoylphosphatidylcholine or dilinoleoylphosphatidylcholine. Amphipathic lipids also can lack phosphorus, such as sphingolipid, glycosphingolipid families, diacylglycerols and b-acyloxyacids.

“Anionic lipid” is any lipid that is negatively charged at physiological pH, including phosphatidylglycerol, cardiolipin, diacylphosphatidylserine, diacylphosphatidic acid, N-dodecanoyl phosphatidylethanolamines, N-succinyl phosphatidylethanolamines, N-glutarylphosphatidylethanolamines, lysylphosphatidylglycerols, and other anionic modifying groups joined to neutral lipids.

“Cationic lipid” carry a net positive charge at a selective pH, such as physiological pH, including N,N-dioleyl-N,N-dimethylammonium chloride (“DODAC”); N-(2,3-dioleyloxy) propyl)-N,N,N-trimethylammonium chloride (“DOTMA”); N,N-distearyl-N,N-dimethylammonium bromide (“DDAB”); N-(2,3-dioleoyloxy) propyl)-N,N,N-trimethylammonium chloride (“DOTAP”); and N-(1,2-dimyristyloxyprop-3-yl)-N,N-dimethyl-N-hydroxyethyl ammonium bromide (“DMRIE”). Additionally, a number of commercial preparations of cationic lipids are available which can be used in the present invention. These include, for example, LIPOFECTIN®, LIPOFECT AMINE®, and TRANSFECTAM®

### Pharmaceutical Compositions

Provided herein are pharmaceutical compositions that comprise a lipid particle encapsulating an active agent. The lipid particle can comprise one or more ionizable lipids; one or more neutral lipids; and one or more PEGylated lipids. These compositions can be buffered at an acidic pH (e.g., a pH of less than 6.5, such as a pH of from 4 to 6.5, or a pH of from 5.0 to 6.5). By buffering at an acidic pH, the delivery efficiency of the compositions can be enhanced as compared to otherwise identical compositions buffered at a pH of 7 or more.

Also provided are pharmaceutical compositions that comprise a lipid particle encapsulating an active agent, the lipid particle comprising: from 20 mol % to 65 mol % one or more ionizable lipids; from 35 mol % to 80 mol % one or more neutral lipids; from greater than 0 mol % to 5 mol % one or more PEGylated lipids; and from 5 mol % to 50 mol % one or more fusogenic oils. By incorporating the one or more fusogenic oils, the delivery efficiency of the compositions can be enhanced as compared to otherwise identical compositions lacking the one or more fusogenic oils. Optionally, these compositions can be buffered at an acidic pH (e.g., a pH of less than 6.5, such as a pH of from 4 to 6.5, or a pH of from 5.0 to 6.5). In some embodiments, these compositions can be buffered at a pH for from 6.5 to less than 6.8, or from 6.5 to less than 7. By buffering at an acidic pH, the delivery efficiency of the compositions can be enhanced as compared to otherwise identical compositions buffered at a pH of 7 or more. In other embodiments, these compositions can be buffered at a pH of from 7 to 7.4.

The lipid particles can have an average diameter of less than 1 micron, such as from from 50 nm to 750 nm, 50 nm to 250 nm, from 50 nm to 200 nm, from 50 nm to 150 nm, or from 50 nm to 100 nm. The lipid particles can have a polydispersity index (PDI) of less than 0.4.

The components of these compositions are described in more detail below.

### Ionizable Lipids

As described above, the compositions described herein can comprise one or more ionizable lipids. An “ionizable lipid” is a lipid that carries a charge that is pH-dependent. The one or more ionizable lipids in the composition described herein can comprise ionizable cationic lipids which carry a positive or neutral carge depending on pH.

Generally, in lipid-based formulations for nucleic acid delivery, either a cationic lipid or an ionizable lipid is used to enable electrostatic interaction with the negatively charged cargo. A cationic lipid is typically defined as a lipid that carries a permanent positive charge(s) that typically comes from a quaternary amine. Examples of a cationic lipids include DOTAP, DOTMA, DDAB, and DODAC. In contrast, ionizable lipids include a chemical moiety, such as a tertiary amine(s), which is positively charged at acidic pH but becomes uncharged at neutral to basic pH. Ionizable lipids can have a pKa value in a biologically relevant range. However, the pKa value of such a lipid is highly dependent on the method used to measure it, resulting in up to 3 units of difference in numerical values for the same lipid. This has been documented in a recent article by Carrasco et al. Communications Biology volume 4, Article number: 956 (2021).

Examples of ionizable lipids are DODMA (N,N-dimethyl-2,3-dioleyloxypropylamine), DODAP, DLinDMA (1,2-dilinoleyloxy-3-dimethylaminopropane), DLinMC3DMA (dilinoleylmethyl-4-dimethylaminobutyrate), DLinKC2DMA (2-dilinoleyl-4-dimethylaminoethyl-[1,3]-dioxolane), ALC-0315 ([(4-hydroxybutyl)azanediyl]di(hexane-6,1-diyl)bis(2-hexyldecanoate)), SM-102 (9-heptadecanyl 8-{(2-hydroxyethyl) [6-oxo-6-(undecyloxy) hexyl]amino}octanoate), Merck-32 (see e.g., WO 2012/018754), Acuitas-5 (see e.g., WO 2015/199952), KL-10 (see e.g., U.S. Patent Application Publication 2012/0295832), C12-200 (see e.g., Love, K T et al., PNAS, 107:1864 (2009)), 3-(N-(N′,N′-dimethylaminoethane)-carbamoyljcholesterol (“DC-Chol”) and the like. Ionizable lipids also include those disclosed in U.S. Pat. Nos. 8,158,601, 9,593,077, 9,365,610, 9,567,296, 9,580,711, and 9,670, 152, International Publication Nos. WO 2012/018754, WO 2015/199952, WO 2019/191780, and U.S. Patent Application Publication Nos. 2012/0295832, 2017/0190661 and 2017/0114010, each of which is incorporated herein by reference in its entirety.

In some embodiments, the one or more ionizable lipids can comprise a lipid headgroup comprising a tertiary amine. In certain embodiments, the one or more ionizable lipids can comprise N,N-dimethyl-2,3-dioleyloxypropylamine (DODMA), [(4-hydroxybutyl)azanediyl]di(hexane-6,1-diyl)bis(2-hexyldecanoate) (ALC-0315); 9-heptadecanyl 8-{(2-hydroxyethyl) [6-oxo-6-(undecyloxy) hexyl]amino}octanoate (SM-102), DLin-MC3-DMA; DLin-KC2-DMA; or any combination thereof.

In some embodiments, the one or more ionizable lipids comprise at least 20 mol % (e.g., at least 25 mol %, at least 30 mol %, at least 35 mol %, at least 40 mol %, at least 45 mol %, at least 50 mol %, at least 55 mol %, or at least 60 mol %) of the total components forming the lipid particle. In some embodiments, the one or more ionizable lipids comprise 65 mol % or less (e.g., 60 mol % or less, 55 mol % or less, 50 mol % or less, 45 mol % or less, 40 mol % or less, 35 mol % or less, 30 mol % or less, or 25 mol % or less) of the total components forming the lipid particle

The one or more ionizable lipids are present in the lipid particle in an amount ranging from any of the minimum values described above to any of the maximum values described above. For example, in some embodiments, the one or more ionizable lipids are present in the lipid particle in an amount of from 20 mol % to 65 mol % (e.g., from 30 mol % to 50 mol %) of the total components forming the lipid particle.

### Neutral Lipids

As described above, the compositions described herein can comprise one or more neutral lipids.

Examples of neutral lipids include phospholipids such as lecithin, phosphatidylethanolamine, lysolecithin, lysophosphatidylethanolamine, phosphatidylserine, phosphatidylinositol, sphingomyelin, egg sphingomyelin (ESM), cephalin, cardiolipin, phosphatidic acid, cerebrosides, dicetylphosphate, distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoylphosphatidylethanolamine (DOPE), palmitoyloleoyl-phosphatidylcholine (POPC), palmitoyloleoyl-phosphatidylethanolamine (POPE), palmitoyloleyol-phosphatidylglycerol (POPG), dioleoylphosphatidylethanolamine 4-(N-maleimidomethyl)-cyclohexane-1-carboxylate (DOPE-mal), dipalmitoyl-phosphatidylethanolamine (DPPE), dimyristoyl-phosphatidylethanolamine (DMPE), distearoyl-phosphatidylethanolamine (DSPE), monomethyl-phosphatidylethanolamine, dimethyl-phosphatidylethanolamine, dielaidoyl-phosphatidylethanolamine (DEPE), stearoyloleoyl-phosphatidylethanolamine (SOPE), lysophosphatidylcholine, dilinoleoylphosphatidylcholine, and mixtures thereof. Other diacylphosphatidylcholine and diacylphosphatidylethanolamine phospholipids can also be used. The acyl groups in these lipids are preferably acyl groups derived from fatty acids having Cio-C24 carbon chains, e.g., lauroyl, myristoyl, palmitoyl, stearoyl, or oleoyl.

Additional examples of neutral lipids include sterols such as cholesterol and derivatives thereof. Non-limiting examples of cholesterol derivatives include polar analogues such as 5a-cholestanol, 5a-coprostanol, cholesteryl-(2′-hydroxy)-ethyl ether, cholesteryl-(4′-hydroxy)-butyl ether, and 6-ketocholestanol; non-polar analogues such as Sa-cholestane, cholestenone, 5a-cholestanone, 5a-cholestanone, and cholesteryl decanoate; and mixtures thereof. In preferred embodiments, the cholesterol derivative is a polar analogue such as cholesteryl-(4′-hydroxy)-butyl ether. Other examples of neutral lipids include nonphosphorous containing lipids such as, e.g, stearylamine, dodecylamine, hexadecylamine, acetyl palmitate, glycerol ricinoleate, hexadecyl stearate, isopropyl myristate, amphoteric acrylic polymers, triethanolamine-lauryl sulfate, alkyl-aryl sulfate polyethyloxylated fatty acid amides, dioctadecyldimethyl ammonium bromide, ceramide, and sphingomyelin.

In some embodiments, the one or more neutral lipids can comprise dipalmitoylphosphatidylcholine (DPPC), dioleoylphosphatidylethanolamine (DOPE), palmitoyloleoylphosphatidylcholine (POPC), egg phosphatidylcholine (EPC), distearoylphosphatidylcholine (DSPC), cholesterol, or any combination thereof.

In some embodiments, the one or more neutral lipids comprise at least 35 mol % (e.g., at least 40 mol %, at least 45 mol %, at least 50 mol %, at least 55 mol %, at least 60 mol %, at least 65 mol %, at least 70 mol %, or at least 75 mol %) of the total components forming the lipid particle. In some embodiments, the one or more neutral lipids comprise 80 mol % or less (e.g., 75 mol % or less, 70 mol % or less, 65 mol % or less, 60 mol % or less, 55 mol % or less, 50 mol % or less, 45 mol % or less, or 40 mol % or less) of the total components forming the lipid particle

The one or more neutral lipids are present in the lipid particle in an amount ranging from any of the minimum values described above to any of the maximum values described above. For example, in some embodiments, the one or more neutral lipids are present in the lipid particle in an amount of from 35 mol % to 80 mol % (30 mol % to 50 mol %) of the total components forming the lipid particle.

### PEGylated Lipids

As described above, the compositions described herein can comprise one or more PEGylated lipids. The one or more PEGylated lipids are useful in that they can reduce or prevent the aggregation of lipid particles.

PEG is a linear, water-soluble polymer of ethylene PEG repeating units with two terminal hydroxyl groups. PEGs are classified by their molecular weights; and include the following: monomethoxypolyethylene glycol (MePEG-OH), monomethoxypolyethylene glycol-succinate (MePEG-S), monomethoxypolyethylene glycol-succinimidyl succinate (MePEG-S-NHS), monomethoxypolyethylene glycol-amine (MePEG-NEh), monomethoxypolyethylene glycol-tresylate (MePEG-TRES), monomethoxypolyethylene glycol-imidazolyl-carbonyl (MePEG-IM), as well as such compounds containing a terminal hydroxyl group instead of a terminal methoxy group (e.g., HO-PEG-S, HO-PEG-S-NHS, HO-PEG-NH2)

Examples of PEG-lipids include, but are not limited to, PEG coupled to dialkyloxypropyls (PEG-DAA), PEG coupled to diacylglycerol (PEG-DAG), PEG coupled to phospholipids such as phosphatidylethanolamine (PEG-PE), PEG conjugated to glycerides forming a glycol, e.g., 1,2-dimyristoyl-sn-glycerol, methoxy-PEG glycol (PEG-DMG), PEG conjugated to ceramides, PEG conjugated to cholesterol, or a derivative thereof, and mixtures thereof. In some examples, the one or more PEGylated lipids can comprise, for example, a PEG-ditetradecylacetamide, a PEG-myristoyl diglyceride, a PEG-diacylglycerol, a PEG dialkyloxypropyl, a PEG-phospholipid, a PEG-ceramide, or any combinations thereof.

The PEG moiety of the PEG-lipid conjugates described herein may comprise an average molecular weight ranging from 550 Daltons to 10,000 Daltons. In certain instances, the PEG moiety has an average molecular weight of from 750 Daltons to 5,000 Daltons (e.g, from 1,000 Daltons to 5,000 Daltons, from 1,500 Daltons to 3,000 Daltons, from 750 Daltons to 3,000 Daltons, from 750 Daltons to 2,000 Daltons). In some embodiments, the PEG moiety has an average molecular weight of 2,000 Daltons or 750 Daltons.

In certain instances, the PEG can be optionally substituted by an alkyl, alkoxy, acyl, or aryl group. The PEG can be conjugated directly to the lipid or may be linked to the lipid via a linker moiety. Any linker moiety suitable for coupling the PEG to a lipid can be used including, e.g., non-ester-containing linker moieties and ester-containing linker moieties. In one embodiment, the linker moiety is a non-ester-containing linker moiety. Suitable non-ester-containing linker moieties include, but are not limited to, amido (—C(O)NH—), amino (—NR—), carbonyl (—C(O)—), carbamate (—NHC(O)O—), urea (—NHC(O)NH—), disulphide (—S—S—), ether (—O—), succinyl (-(0)CCH2CH2C(0)-), succinamidyl (—NHC(0) CH2CH2C(0) NH—), ether, disulphide, as well as combinations thereof (such as a linker containing both a carbamate linker moiety and an amido linker moiety). In some embodiments, a carbamate linker is used to couple the PEG to the lipid. In other embodiments, an ester-containing linker moiety can be used to couple the PEG to the lipid. Suitable ester-containing linker moieties include, e.g., carbonate (—OC(O)O—), succinoyl, phosphate esters (—O—(O)POH—O—), sulfonate esters, and combinations thereof.

The term “diacylglycerol” or “DAG” includes a compound having 2 fatty acyl chains, R¹ and R², both of which have independently between 2 and 30 carbons bonded to the 1- and 2-position of glycerol by ester linkages. The acyl groups can be saturated or have varying degrees of unsaturation. Suitable acyl groups include, but are not limited to, lauroyl (C12), myristoyl (Ci₄), palmitoyl (Ci6), stearoyl (Cis), and icosoyl (C20). In preferred embodiments, R¹ and R² are the same, i.e., R¹ and R² are both myristoyl (i.e., dimyristoyl), R¹ and R² are both stearoyl (i.e., distearoyl).

The term “dialkyloxyalkyl” or “DAA” includes a compound having 2 alkyl chains, R and R′, both of which have independently between 2 and 30 carbons. The alkyl groups can be saturated or have varying degrees of unsaturation.

Examples of PEG-DAA conjugates include PEG-didecyloxypropyl (C10), a PEG-dilauryloxypropyl (C12), a PEG-dimyristyloxypropyl (C14), a PEG-dipalmityloxypropyl (C16), and PEG-distearyloxypropyl (C18). In some of these embodiments, the PEG can have an average molecular weight of 750 or 2,000 Daltons. In certain embodiments, the terminal hydroxyl group of the PEG can be substituted with a methyl group.

In addition to the foregoing, other hydrophilic polymers can be used in place of PEG. Examples of suitable polymers that can be used in place of PEG include, but are not limited to, polyvinylpyrrolidone, polymethyloxazoline, polyethyloxazoline, polyhydroxypropyl methacrylamide, polymethacrylamide and polydimethylacrylamide, polylactic acid, polyglycolic acid, and derivatized celluloses such as hydroxymethylcellulose or hydroxyethylcellulose.

In some embodiments, the one or more PEGylated lipids comprise greater than 0 mol % (e.g., at least 0.5 mol %, at least 1 mol %, at least 1.5 mol %, at least 2 mol %, at least 2.5 mol %, at least 3 mol %, at least 3.5 mol %, at least 4 mol %, or at least 4.5 mol %) of the total components forming the lipid particle. In some embodiments, the one or more PEGylated lipids comprise 5 mol % or less (e.g., 4.5 mol % or less, 4 mol % or less, 3.5 mol % or less, 3 mol % or less, 2.5 mol % or less, 2 mol % or less, 1.5 mol % or less, 1 mol % or less, or 0.5 mol % or less) of the total components forming the lipid particle

The one or more PEGylated lipids are present in the lipid particle in an amount ranging from any of the minimum values described above to any of the maximum values described above. For example, in some embodiments, the one or more PEGylated lipids are present in the lipid particle in an amount of from greater than 0 mol % to 5 mol % of the total components forming the lipid particle.

### Fusogenic Oils

In some embodiments, the compositions described herein can comprise one or more fusogenic oils.

In some embodiments, the fusagenic oil can comprise a C12-C40 hydrocarbon (e.g., a C12 hydrocarbon, a C13 hydrocarbon, a C14 hydrocarbon, a C15 hydrocarbon, a C16 hydrocarbon, a C17 hydrocarbon, a C18 hydrocarbon, a C19 hydrocarbon, a C20 hydrocarbon, a C21 hydrocarbon, a C22 hydrocarbon, a C23 hydrocarbon, a C24 hydrocarbon, a C25 hydrocarbon, a C26 hydrocarbon, a C27 hydrocarbon, a C28 hydrocarbon, a C29 hydrocarbon, a C30 hydrocarbon, a C31 hydrocarbon, a C32 hydrocarbon, a C33 hydrocarbon, a C34 hydrocarbon, a C35 hydrocarbon, a C36 hydrocarbon, a C37 hydrocarbon, a C38 hydrocarbon, a C39 hydrocarbon, or a C40 hydrocarbon). In some cases, the C12-C40 hydrocarbon can comprise an alkyl or alkylene chain. Alkylene chains can include one or more double bonds (e.g., from one to five double bonds, or from one to three double bonds). In some embodiments, the C12-C40 hydrocarbon can comprise an alkylene chain, optionally comprising a least one cis-double bond. In some embodiments, the fusogenic oil can comprise fewer than three rings (e.g., fewer than two rings, or no rings).

In some examples, the fusogenic oil can comprise squalene, squalane, pristane, pristene, farnesene, farnesane, retinol, phytol, a carotene, a tocopherol, a tocotrienol, phytomenadione, menaquinone, where valence permits esters thereof, or a combination thereof. In certain embodiments, the fusogenic oil can comprise squalene.

When present in the compositions described herein, in some embodiments, the one or more fusogenic oils comprise at least 10 mol % (e.g., at least 15 mol %, at least 20 mol %, at least 25 mol %, at least 30 mol %, or at least 35 mol %) of the total components forming the lipid particle. When present in the compositions described herein, the one or more fusogenic oils comprise 40 mol % or less (e.g., 35 mol % or less, 30 mol % or less, 25 mol % or less, 20 mol % or less, or 15 mol % or less) of the total components forming the lipid particle

The one or more fusogenic oils are present in the lipid particle in an amount ranging from any of the minimum values described above to any of the maximum values described above. For example, in some embodiments, the one or more fusogenic oils can be present in the lipid particle in an amount of from 10 mol % to 40 mol % of the total components forming the lipid particle.

When present in the compositions described herein, in some embodiments, the fusogenic oil and the one or more PEGylated lipids can be present in the lipid particles at a molar ratio of at least 5:1 (e.g., at least 10:1, or at least 15:1). When present in the compositions described herein, in some embodiments, the fusogenic oil and the one or more PEGylated lipids can be present in the lipid particles at a molar ratio of 20:1 or less (e.g., 15:1 or less, or 10:1 or less).

The fusogenic oil and the one or more PEGylated lipids can be present in the lipid particles at a molar ratio ranging from any of the minimum values described above to any of the maximum values described above. For example, in some embodiments, the fusogenic oil and the one or more PEGylated lipids can be present in the lipid particles at a molar ratio of from 5:1 to 20:1.

When present in the compositions described herein, in some embodiments, the fusogenic oil and the one or more ionizable lipids can be present in the lipid particles at a molar ratio of at least 0.25:1 (e.g., at least 0.5:1, or at least 0.75:1). When present in the compositions described herein, in some embodiments, the fusogenic oil and the one or more ionizable lipids can be present in the lipid particles at a molar ratio of 1:1 or less (e.g., 0.75:1 or less, or 0.5:1 or less).

The fusogenic oil and the one or more ionizable lipids can be present in the lipid particles at a molar ratio ranging from any of the minimum values described above to any of the maximum values described above. For example, in some embodiments, the fusogenic oil and the one or more ionizable lipids can be present in the lipid particles at a molar ratio of from 0.25:1 to 1:1.

### Active Agents

The active agent encapsulated in the lipid particles can comprise any suitable active agent, such as a small molecule therapeutic agent, a diagnostic agent, a peptide, a protein, an antibody, or a nucleic acid.

In certain embodiments, the active agent can comprise a nucleic acid.

A nucleic acid (NA, e.g., a polynucleotide or oligonucleotide) encoding a peptide may be used to produce an antigenic peptide in vitro. The NA may be, e.g., DNA, cDNA, PNA, CNA, RNA, either single- and/or double-stranded, or native or stabilized forms of polynucleotides, such as e.g., polynucleotides with a phosphorothioate backbone, or combinations thereof and it may or may not contain introns so long as it codes for the peptide. In one embodiment in vitro translation is used to produce the peptide. Many exemplary systems exist that one skilled in the art could utilize.

In some embodiments, the active agent can comprise a mRNA or an expression vector capable of expressing a polypeptide. Expression vectors for different cell types are well known in the art and can be selected without undue experimentation. Generally, the DNA is inserted into an expression vector, such as a plasmid, in proper orientation and correct reading frame for expression, if necessary, the DNA may be linked to the appropriate transcriptional and translational regulatory control nucleotide sequences recognized by the desired host (e.g., bacteria), although such controls are generally available in the expression vector. The vector is then introduced into the host bacteria for cloning using standard techniques (see, e.g., Sambrook et al. (1989) MOLECULAR CLONING, A LABORATORY MANUAL, Cold Spring Harbor Laboratory, NY).

The term “nucleic acid encoding a polypeptide” encompasses a NA that includes only coding sequences for the polypeptide as well as a NA that includes additional coding and/or non-coding sequences. NA can be in the form of RNA or in the form of DNA. DNA includes cDNA, genomic DNA, and synthetic DNA; and can be double-stranded or single-stranded, and if single stranded can be the coding strand or non-coding (anti-sense) strand.

The NA may comprise the coding sequence for the peptide, either an antibody or an antigen, fused in the same reading frame to a polynucleotide which aids, for example, in expression and/or secretion of a polypeptide from a host cell (e.g, a leader sequence which functions as a secretory sequence for controlling transport of a polypeptide from the cell). The polypeptide having a leader sequence is a pre-protein and can have the leader sequence cleaved by the host cell to form the mature form of the polypeptide.

A NA sequence encoding a polypeptide of interest would be constructed by chemical synthesis using an oligonucleotide synthesizer. Such oligonucleotides can be designed based on the amino acid sequence of the desired polypeptide and selecting those codons that are favored in the host cell in which the recombinant polypeptide of interest is produced. Standard methods can be applied to synthesize an isolated polynucleotide sequence encoding an isolated polypeptide of interest. An oligomer containing a NA sequence coding for the particular isolated polypeptide can be synthesized. For example, several small oligonucleotides coding for portions of the desired polypeptide can be synthesized and then ligated. The individual oligonucleotides typically contain 5′ or 3′ overhangs for complementary assembly.

Once assembled (e.g., by synthesis, site-directed mutagenesis, or another method), the polynucleotide sequences encoding a particular isolated polypeptide of interest is inserted into an expression vector and optionally operatively linked to an expression control sequence appropriate for expression of the protein in a desired host. Proper assembly can be confirmed by nucleotide sequencing, restriction mapping, and expression of a biologically active polypeptide in a suitable host. As well known in the art, in order to obtain high expression levels of a transfected gene in a host, the gene can be operatively linked to transcriptional and translational expression control sequences that are functional in the chosen expression host.

Recombinant expression vectors may be used to amplify and express DNA encoding antibodies or antigenic peptides. Recombinant expression vectors are replicable DNA constructs having synthetic or cDNA-derived DNA fragments operatively linked to suitable transcriptional or translational regulator elements derived from mammalian, microbial, viral or insect genes. A transcriptional unit generally comprises an assembly of a genetic element or elements having a regulatory role in gene expression, for example, transcriptional promoters or enhancers, a structural or coding sequence which is transcribed into mRNA and translated into protein, and appropriate transcription and translation initiation and termination sequences, as described in detail herein. Such regulatory elements can include an operator sequence to control transcription. Generally, operatively linked means contiguous, and in the case of secretory leaders, means contiguous and in reading frame. Structural elements intended for use in yeast expression systems include a leader sequence enabling extracellular secretion of translated protein by a host cell. Alternatively, where recombinant protein is expressed without a leader or transport sequence, it can include an N-terminal methionine residue. This residue can optionally be subsequently cleaved from the expressed recombinant protein to provide a final product.

“Ribonucleic acid” or “RNA” refers to a polymer containing at least two ribonucleotides. “Ribonucleotides” contain a sugar ribose, a base, and a phosphate group. Nucleotides are linked together through the phosphate groups. “Bases” include purines and pyrimidines, which further include natural compounds adenine, thymine, guanine, cytosine, uracil, inosine, and natural analogs, and synthetic derivatives of purines and pyrimidines, which include, but are not limited to, modifications which place new reactive groups such as, but not limited to, amines, alcohols, thiols, carboxylates, and alkyl halides.

RNA may be in the form of oligonucleotide RNA, tRNA (transfer RNA), snRNA (small nuclear RNA), rRNA (ribosomal RNA), mRNA (messenger RNA), antisense RNA, siRNA (small interfering RNA), self-replicating RNA, ribozymes, chimeric sequences, or derivatives of these groups.

The RNA can include (in addition to any 5′ cap structure) one or more nucleotides having a modified nucleobase, including m5C (5-methylcytidine), mSU (5-methyluridine), m6A (N6-methyladenosine), s2U (2-thiouridine), Um (2′-0-methyluridine), mlA (1-methyladenosine); m2A (2-methyladenosine); Am (2′-0-methyladenosine); ms2m6A (2-methylthio-N6-methyladenosine); 16A (N6-isopentenyladenosine); ms216A (2-methylthio-N6isopentenyladenosine); io6A (N6-(cis-hydroxyisopentenyl)adenosine); ms2io6A (2-methylthio-N6-(cis-hydroxyisopentenyl) adenosine); g6A (N6-glycinylcarbamoyladenosine); t6A (N6-threonyl carbamoyladenosine); ms2t6A (2-methylthio-N6-threonyl carbamoyladenosine); m6t6A (N6-methyl-N6-threonylcarbamoyladenosine); hn6A(N6.-hydroxynorvalylcarbamoyl adenosine); ms2hn6A (2-methylthio-N6-hydroxynorvalyl carbamoyladenosine); Ar(p) (2′-0-ribosyladenosine (phosphate)); I (inosine); ml 1 (1-methylinosine); m′lm (1,2′-0-dimethylinosine); m3C (3-methylcytidine); Cm (2T-0-methylcytidine); s2C (2-thiocytidine); ac4C (N4-acetylcytidine); f5C (5-fonnylcytidine); m5Cm (5,2-O-dimethylcytidine); ac4Cm (N4acetyl2TOmethylcytidine); k2C (lysidine); mIG (l-methylguanosine); m2G (N2-methylguanosine); m7G (7-methylguanosine); Gm (2′-0-methylguanosine); m22G (N2,N2-dimethylguanosine); m2Gm (N2,2′-0-dimethylguanosine); m22Gm (N2,N2,2′-0-trimethvlguanosine); Gr(p) (2′-0-ribosylguanosine (phosphate)); yW (wybutosine); o2yW (peroxywybutosine); OHyW (hydroxywybutosine); OHyW* (undermodified hydroxywybutosine); imG (wyosine); mimG (methylguanosine); Q (queuosine); oQ (epoxyqueuosine); galQ (galtactosyl-queuosine); manQ (mannosyl-queuosine); preQo (7-cyano-7-deazaguanosine); preQi (7-aminomethyl-7-deazaguanosine); G* (archaeosine); D (dihydrouridine); m5Um (5,2′-0-dimethyluridine); s4U (4-thiouridine); m5s2U (5-methyl-2-thiouridine); s2Um (2-thio-2′-0-methyluridine); acp3U (3-(3-amino-3-carboxypropyl)uri dine); ho5U (5-hydroxyuridine); mo5U (5-methoxyuridine); cmo5U (uridine 5-oxyacetic acid); mcmo5U (uridine 5-oxyacetic acid methyl ester); chm5U (5-(carboxyhydroxymethyl)uridine)); mchm5U (5-(carboxyhydroxymethyl)uridine methyl ester); mcm5U (5-methoxycarbonyl methyluridine); mcm5Um (S-methoxycarbonylmethyl-2-0-methyluridine); mcm5s2U (5-methoxycarbonylmethyl-2-thiouridine); nm5s2U (5-aminomethyl-2-thiouridine); mnm5U (5- methylaminomethyluridine); mnm5s2U (5-methylaminomethyl-2-thiouridine); mnm5se2U (5- methylaminomethyl-2-selenouridine); ncm5U (5-carbamoylmethyl uridine); ncm5Um (5- carbamoylmethyl-2′-0-methyluridine); cmnm5U (5-carboxymethylaminomethyluridine); cnmm5Um (5-carboxymethylaminomethyl-2-L-0-methyluridine); cmnm5s2U (5-carboxymethylaminomethyl-2-thiouridine); m62A (N6,N6-dimethyladenosine); Tm (2′-0-methylinosine); m4C (N4-methylcytidine); m4Cm (N4,2-0-dimethylcytidine); hm5C (5-hydroxymethylcytidine); m3U (3-methyluridine); cm5U (5-carboxymethyluridine); m6Am (N6,T-0-dimethyladenosine); rn62Am (N6,N6,0-2-trimethyladenosine); m2′7G (N2,7-dimethylguanosine); m2′2′7G (N2,N2,7-trimethylguanosine); m3Um (3,2T-0-dimethyluridine); m5D (5-methyldihydrouridine); f5Cm (5-formyl-2′-0-methylcytidine); mlGm (1,2′-0- dimethylguanosine); m′Am (1, 2-O-dimethyl adenosine) irinomethyluridine); tm5s2U (S- taurinomethyl-2-thiouridine)); imG-14 (4-demethyl guanosine); imG2 (isoguanosine); or ac6A (N6-acetyladenosine), hypoxanthine, inosine, 8-oxo-adenine, 7-substituted derivatives thereof, dihydrouracil, pseudouracil, 2-thiouracil, 4-thiouracil, 5-aminouracil, 5-(Cl-C6)-alkyluracil, 5-methyluracil, 5-(C2-C6)-alkenyluracil, 5-(C2-C6)-alkynyluracil, 5-(hydroxymethyl)uracil, 5-chlorouracil, 5-fluorouracil, 5-bromouracil, 5-hydroxy cytosine, 5-(C1-C6)-alkylcytosine, 5-methylcytosine, 5-(C2-C6)-alkenylcytosine, 5-(C2-C6)-alkynylcytosine, 5-chlorocytosine, 5-fluorocytosine, 5-bromocytosine, N2-dimethylguanine, 7-deazaguanine, 8-azaguanine, 7-deaza- 7-substituted guanine, 7-deaza-7-(C2-C6)alkynylguanine, 7-deaza-8-substituted guanine, 8-hydroxyguanine, 6-thioguanine, 8-oxoguanine, 2-aminopurine, 2-amino-6-chloropurine, 2,4- diaminopurine, 2,6-diaminopurine, 8-azapurine, substituted 7-deazapurine, 7-deaza-7-substituted purine, 7-deaza-8-substituted purine, or an abasic nucleotide.

The RNA may optionally comprise one or more UNA molecules, e.g., as disclosed in U.S. Pat. Nos. 8,314,227, 9,051,570, 9,303,260, 9,297,009, and 9,340,789, and U.S. Patent Publication No. 2016/0168567, incorporated herein in their entirety.

The RNA or self-replicating RNA can include one or more modified pyrimidine nucleobases, such as pseudouridine and/or 5-methylcytosine residues.

The RNA may include a 5′ cap comprising a 7′-methylguanosine, and the first 1, 2 or 3 5′ ribonucleotides may be methylated at the 2′ position of the ribose. The RNA can contain a 5′ trinucleotide cap structure as described by Tanis, et al., U.S. application Ser. No. 15/788,742, filed Oct. 19, 2017, herein incorporated by reference in its entirety.

Natural RNA have a phosphate backbone, RNA as described herein may contain other types of backbones and bases including peptide nucleic acids, phosphothionates, phosphoramidate, phosphorothioate, and/or methylphosphonate linkages.

“Antisense” is a polynucleotide that interferes with the function of DNA and/or RNA. This may result in suppression of expression.

“Gene” refers to a nucleic acid (e.g., DNA) sequence that comprises coding sequences necessary for the production of a polypeptide or precursor. The polypeptide can be encoded by a full length coding sequence or by any portion of the coding sequence so long as the desired activity or functional properties (e.g, enzymatic activity, ligand binding, signal transduction, and the like) of the full-length polypeptide or fragment thereof are retained.

### Methods of Use

These compositions can be prepared as described herein or elsewhere, and can be administered by a variety of routes, depending upon whether local or systemic treatment is desired and upon the area to be treated. Administration may be topical (including transdermal, epidermal, ophthalmic and to mucous membranes including intranasal, vaginal and rectal delivery), pulmonary (e.g., by inhalation or insufflation of powders or aerosols, including by nebulizer; intratracheal or intranasal), oral, or parenteral. Parenteral administration includes intravenous, intraarterial, subcutaneous, intraperitoneal intramuscular or injection or infusion; or intracranial, (e.g., intrathecal or intraventricular, administration). Parenteral administration can be in the form of a single bolus dose, or may be, for example, by a continuous perfusion pump. In some embodiments, the compounds provided herein, or a pharmaceutically acceptable salt thereof, are suitable for parenteral administration. In some embodiments, the compounds provided herein are suitable for intravenous administration. In some embodiments, the compounds provided herein are suitable for oral administration. In some embodiments, the compounds provided herein are suitable for topical administration.

Pharmaceutical compositions and formulations for topical administration may include, but are not limited to, transdermal patches, ointments, lotions, creams, gels, drops, suppositories, sprays, liquids and powders. Conventional pharmaceutical carriers, aqueous, powder or oily bases, thickeners and the like may be necessary or desirable. In some embodiments, the pharmaceutical compositions provided herein are suitable for parenteral administration. In some embodiments, the pharmaceutical compositions provided herein are suitable for intravenous administration. In some embodiments, the pharmaceutical compositions provided herein are suitable for oral administration. In some embodiments, the pharmaceutical compositions provided herein are suitable for topical administration.

The compositions described herein can be used to deliver one or more active agents to cells (e.g., in vivo, ex vivo, or in vitro). Accordingly, provided herein are method of delivering an active agent to a cell (e.g., in vivo, ex vivo, or in vitro) that comprise contacting the cell with a composition described herein. Also provided are methods for in vivo delivery of an active agent to a cell, said method comprising administering to a mammalian subject (e.g., a human) a composition described herein. In some embodiments, the administration can comprise systemic administration (eg., intravenous injection or infusion).

Reference will now be made in detail to the present exemplary embodiments, examples of which are illustrated in the accompanying drawings. Other embodiments will be apparent to those skilled in the art from consideration of the specification and practice disclosed herein. The embodiments are further explained in the following examples. These examples do not limit the scope of the claims, but merely serve to clarify certain embodiments. It is intended that the specification and examples be considered as exemplary only, with a true scope and spirit being indicated by the following claims.

### EXAMPLES

The invention will be described in greater detail by way of specific examples. The following examples are offered for illustrative purposes, and are not intended to limit the invention in any manner. Those of skill in the art will readily recognize a variety of non-critical parameters which can be changed or modified to yield essentially the same results.

### Example 1: A Squalene-Based Nanoemulsion for Therapeutic Delivery of TLR Agonists

### Overview

Agonists for toll-like receptors (TLRs) have shown promising activities against cancer. In this example, a squalene-based nanoemulsion was loaded with resiquimod, a TLR7/8 agonist for therapeutic delivery. When combined with SD-101, a CpG-containing TLR9 agonist, strong anti-tumor activity was observed in MC38 murine colon carcinoma model. The treatment induced PD-L1 upregulation in tumors, suggesting a potential therapeutic synergy with immune checkpoint inhibitors.

### Background

Toll-like receptors (TLRs) play critical roles in immune responses by recognizing pathogen-associated molecule patterns (PAMP) followed by inducing cytokine production and activating adaptive immunity. TLRs are expressed either on the plasma membrane (TLR1/2/4/5/6/10) or in endosomes (TLR3/7/8/9) in antigen-presenting cells (APCs) such as dendritic cells and macrophages. TLR activation leads to the MyD88/NF-κB pathway induction and naïve T cell repertoires activation in adaptive immune responses[1]. Studies have shown that endosomal TLR agonists worked effectively as adjuvants in cancer vaccines due to their strong immunostimulatory activities [2]. Endosomal TLR agonists have been shown to activate plasmacytoid dendritic cells (pDCs) and cytotoxic T lymphocytes (CTLs), enhancing T cell-mediated immunity. Three agents with TLR agonist activity have been approved by FDA for cancer treatments including bacillus Calmette-Guerin (TLR2&4 agonists mixture), monophosphoryl lipid A (TLR2/4 agonists mixture), and imiquimod (TLR7 agonist)[2]. Overall the clinical efficacy of TLR agonists has been mixed [3-4]. Intra-tumoral injection has been investigated in recent CpG TLR9 agonists. However, this mode of administration is difficult in clinical practice for most solid tumors.

Strong innate and adaptive immune system activation and promising antitumor efficacy can be achieved using TLR7/8 and TLR9 combinations [5-7]. Synergistic cytokine release and antibody productions were observed using a Schistosoma japonicum DNA vaccine, containing a combination of TLR 7/8 and TLR9 agonists [6]. Another study has shown significant tumor suppression and synergistic IFN-γ secretion by TLR7/8/9 combination treatments[5-8]. However, these results on duo-TLR activation lacked an efficient platform for the delivery of TLR agonists.

Resiquimod (R848) is a TLR7/8 agonist that has shown antitumor activity in murine tumor models[9-12]. However, due to its limited solubility, an injectable formulation is needed for the clinical application of R848. Oil-in-water nanoemulsions (NE) are effective delivery systems for hydrophobic drugs[13-16]. NE consists of an oil core stabilized by surfactants, where the oil core could work as an efficient reservoir for poorly water-soluble drugs [17]. In addition, squalene-based NE has been shown as an efficient vaccine adjuvant by adaptive immunity activation[18]. Squalene-based NE vaccine adjuvants MF59 and AddaVax have been administered to more than 100 million people in more than 30 countries, in both seasonal and pandemic influenza vaccines. It is also noticed that the squalene core in NE, which was originally derived from shark liver oil, has been reported to potentiate both immune responses and antitumor efficacy[19].

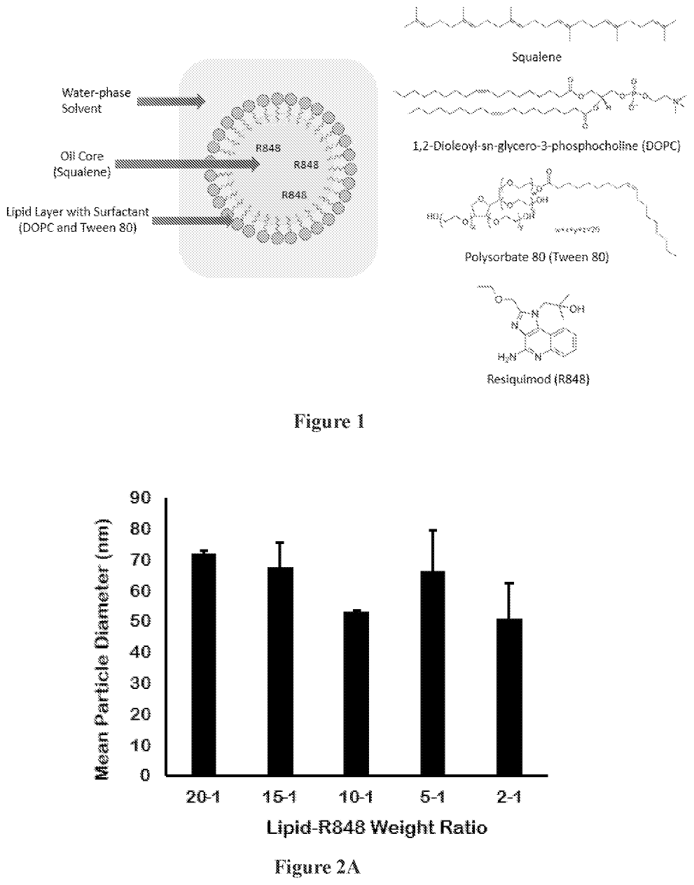

In this study, we developed a squalene-based NE using 1,2-dioleoyl-sn-glycero-3-phosphocholine (DOPC) and polysorbate 80 (Tween 80) as surfactants to encapsulate R848 (FIG. 1). Here, the squalene core was utilized not only to potentiate immune response but also to dissolve R848, which is a poor water-soluble molecule. We evaluated the R848 encapsulation efficiency in squalene-based NE and optimized drug-loading capacity by adjusting lipid-to-drug ratio. The R848-loaded squalene-based NE also showed long-term stability up to 1 month at 4° C.

Besides R848, we incorporated SD-101 as the other component to boost immunity activation. SD-101 is a second-generation TLR9 agonist containing cytidine-phospho-guanosine (CpG) dinucleotides. This class-C oligonucleotide stimulates TLR9, majorly expressed on pDCs and potentiates both innate and adaptive immune responses[20]. Furthermore, SD-101 has been shown to have promising antitumor efficacy in combination with immune checkpoint inhibitors or radiation therapies in clinical trials[20-22]. Our study demonstrated the synergistic antitumor activity through adaptive immune stimulation by R848-loaded NE and SD-101. The combination treatment of R848-loaded NE and SD-101 also showed upregulation of Pdl1 mRNA level in a mouse model, suggesting a therapeutic strategy based on combining TLR activation and PDL1 targeting.

### Materials and Methods

### Materials.

Squalene was obtained from Sigma-Aldrich (St. Louis, MO, USA). 1,2-dioleoyl-sn-glycero-3-phosphocholine (DOPC) was purchased from Avanti Polar Lipids (Birmingham, AL). Resiquimod (R848) was purchased from MedChemExpress (Monmouth Junction, NJ), and SD-101 was synthesized by Alpha DNA (Montreal, Quebec, Canada). TNF-α, IL-6, and IL-12p70 mouse uncoated ELISA kits and high-capacity cDNA reverse transcription kit were purchased from Invitrogen (Waltham, MA). SsoAdvanced™ Universal SYBR® Green Supermix was purchased from Bio-Rad Laboratories (Hercules, CA). Real-time PCR pre-designed primers for murine Akt1, Bcl2, Hif1a, Pd1 (Cd274), Calreticulin, Hmgb1, and Actb were obtained from Sigma-Aldrich (St. Louis, MO). Primers for murine Cd3e, CD4, CD8a, Foxp3 and Ifng were designed and were synthesized by ThermoFisher Scientific (Waltham, MA). Polysorbate 80 (Tween 80) and all other chemicals and buffers otherwise stated were purchased from Fisher Scientific (Hampton, NH).

### R848-NE Formulation and Characterization.

Squalene-based NE were prepared by hand-rapid injection of oil-lipid mixture into phosphate buffered saline (PBS). Squalene, DOPC, and Tween 80 were prepared at a molar ratio of 1/1/1 in ethanol. R848 was then added to the lipid-ethanol solution, maintaining lipid to R848 ratio at 10:1 (w/w). The final total lipid concentration of the nanoemulsion was 8 mg/mL, and the final R848 concentration was 0.8 mg/mL. R848-loaded NE (R848 NE) with lipid to R848 weight ratio of 20:1, 15:1, 10.1, 5:1, and 2:1 were developed to optimize R848 loading capacity in squalene-based nanoemulsion based on particle sizes and R848 solubility. Particle sizes were measured by dynamic light scattering (DLS) using a NICOMP NANO ZLS Z3000 (Entegris, Billerica, MA). Empty nanoemulsion (empty NE) was generated using similar procedures without adding R848. Empty NE and R848 NE were stored at 4° C. prior to characterization and long-term stability.

Sepharose CL-4B size exclusion chromatography was performed to examine the encapsulation efficiency of R848 within the squalene nanoemulsions. R848 concentrations were determined by UV-Vis spectrometry at 320 nm using a NanoDrop 2000 spectrophotometer [23]. Resiquimod loading efficiency was determined by the formula:

$$Loading Efficiency \% = \frac{UV absprbance at 320 nm of fractions 3 - 6}{UV absorbance at 320 nm of the entire fractions} \times 100 \%$$

### Cell Culture.

RAW 264.7 murine macrophage cell line and MC38 murine coloractal carcinoma cell line were kind gifts given by Dr. Peixuan Guo and Dr. Christopher Coss at The Ohio State University College of Pharmacy, respectively. RAW 264.7 and MC38 were grown in DMEM supplemented with 10% FBS and 1x antibiotic-antimycotic and maintained at 37° C. under a humidified atmosphere containing 5% C02.

### In Vitro Macrophage Stimulation Imaging.

RAW 264.7 cells were seeded at a density of 1.5×10⁵ cells/well in 24-well plates 24 hours prior to treatments. Cells were treated with empty NE, R848, SD-101, R848 NE, or R848 NE/SD-101 combination for 24 hours. R848 was treated at 50 μM either independently, in nanoemulsion, or in combination with SD-101. SD-101 was treated at 300 nM individually or in combination. The morphological changes of RAW 264.7 cells were visualized under 200× brightfield by a Nikon Eclipse Ti-S microscope (Nikon, Tokyo, Japan) after 24-hour incubation.

### In Vitro Cytokine Induction Evaluation by Enzyme-Linked Immunosorbent Assay (ELISA).

RAW 264.7 cells were seeded at a density of 3×10⁵ cells/well in 6-well plates 24 hours prior to treatments. Cells were treated with empty NE, R848, SD-101, R848 NE, or R848 NE/SD-101 combination for 24 hours. R848 was treated at 50 μM either individually, in nanoemulsion or in combination. SD-101 was treated at 300 nM either individually or in combination. Supernatant was collected and stored at −80° C. prior to cytokine quantification by ELISA. Supernatants were 6-fold pre-diluted by PBS, and TNF-a, IL-6, and IL-12p70 concentrations were measured by mouse uncoated ELISA kits per manufacturer's protocol.

### In Vivo Antitumor Efficacy.

MC38 murine colorectal syngeneic model was generated by subcutaneously inoculating C57BL/6N mice (obtained from Charles River Labortories) with 0.5×10⁶ cells per mouse on the right flack. Treatments were initiated once tumors reached approximately ˜100 mm³. Mice (n=5) were intraperitoneally treated with saline, 4 mg/kg R848 NE, 2 mg/kg SD-101, or R848 NE/SD-101 combination (4 mg/kg R848 NE and 2 mg/kg SD-101). All treatment solutions were prepared in PBS. Mice were dosed every 3 days for 4 doses. Tumor growth and body weight were monitored and the tumor volumes were calculated according to the formula:

$$Tumor Volume = \frac{Length \times {Width}^{2}}{2}$$

All animal studies were reviewed and approved by The Ohio State University Institutional Laboratory Animal Care and Use Committee (IACUC). All mice were euthanized on day 10, 6 hours after the fourth dose to peak the serum cytokine concentrations. Whole blood was collected through cardiac puncture. Tumor and spleen tissues were harvested and weighed for comparison. Spleen weights were normalized to individual body weights for comparison between treatment groups. Tissues and sera were stored at −80° C. prior to in vivo cytokine and gene regulation studies. Tumor growth inhibition (% TGI) on day 10 was determined by the formula:

$$\% TGI = \frac{1 - ( {T}_{10} / {T}_{0} ) / ( {C}_{10} / {C}_{0} )}{1 - {C}_{0} / {C}_{10}} \times 100 \%$$

Where T₁₀ stands for average tumor volume of treatment group at day 10, T₀ stands for average tumor volume of treatment group at day 0, Cio stands for average tumor volume of control group at day 10, and Co stands for average tumor volume of control group at day 0. % TGI>50% was considered meaningful.

### In Vivo Cytokine Measurement.

Mouse sera were collected by placing whole blood at room temperature for 30 minutes followed by 2000×g centrifugation for 20 minutes. Samples were collected and stored at −80° C. prior to cytokine quantification. Murine TNF-αc, IL-6, and IL12p70 cytokine concentrations were determined by ELISA per manufacturer's protocol.

In Vivo Gene Regulation by Real-Time qPCR.

Tumor and spleen tissues were homogenized in TRI reagent using probe sonication, and total RNA was extracted per manufacturer's protocol. cDNA was prepared using High-Capacity cDNA Reverse Transcription Kit per manufacturer's protocol. Real-time PCR was conducted on a QuantStudio 7 Flex Real-Time PCR System with target genes Akt1, Bcl2, and Pdl1 in spleen tissue samples, and Akt1, Bcl2, Hif1a, Pdl1, Calreticulin, Hmgb1 in tumor tissue samples. All genes were normalized to Actb as the housekeeping gene. The relative amount of RNA level was calculated and compared according to the 2^{-ΔΔCt} method[24,25].

### Statistical Analysis.

All studies were done in triplicate. Data are presented as means±standard deviations unless otherwise indicated. Statistical analysis will be conducted using Microsoft Excel. One-way ANOVA will be used to determine variances in means between two or more treatment groups. A p-value of 0.05 was selected as the cutoff for statistical significance.

### Results and Discussion

### Particle Characterization.

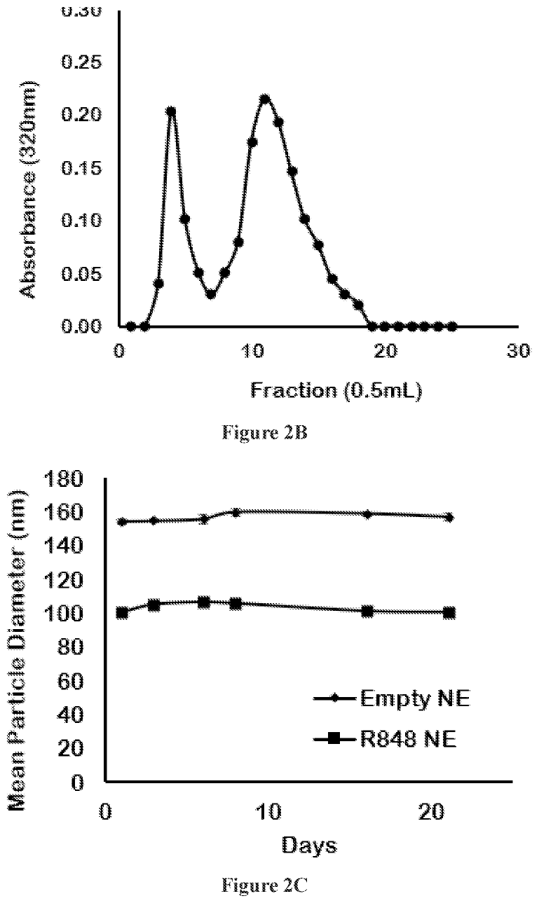

As a TLR7/8 agonist, resiquimod has been demonstrated to have great potency for cancer immunotherapy compared with other imidazoquinoline-analogs[26]. However, the tolerance induction and adverse effects limit its development as a strong candidate for a market drug. Several studies utilized polymer-based nanoparticles such as polylactic acid (PLA) or β-cyclodextrin to carry R848 to overcome these limitations[27-29]. Nonetheless, the disadvantages of polymeric nanoparticles include an toxic degradation for polymer materials and self-aggregation[30]. In our study, we utilized a squalene-based oil-in-water nanoemulsion as a carrier for R848 (FIG. 1). The neutrally charged components (squalene, DOPC, and Tween 80) could eliminate the cytotoxicity carried out by cationic lipid nanoparticles and polymer nanoparticles[31]. R848 NE was approximately 50-100 nm in size (FIG. 2A). There was no significant changes in particle sizes among lipid-to-R848 weight ratio through 20:1 to 2:1, indicating the addition of R848 not affecting the structural stability of nanoemulsions. However, R848 precipitation due to insufficient oil phase was observed in R848 NE samples with 2:1 lipid-to-R848 weight ratio after storing overnight at 4° C. and with 5:1 lipid-to-R848 weight ratio after 1-week storage at 4° C. (data not shown). Therefore, R848 NE with 10:1 lipid-to-R848 weight ratio was selected for further studies with maximized R848 loading amount. Size exclusion chromatography using a Sepharose CL-4B gel column (FIG. 2B) showed 35.9%±0.53% of R848 within the NE-encapsulated fractions, which was much higher than another liposomal formulation of R848 with only 7% encapsulation efficiency[32]. The result indicated that oil-in-water nanoemulsion worked better than liposomes in encapsulating the poor water-soluble agent. We further demonstrated that both empty NE and R848 NE exhibited high colloidal stability under storage at 4° C. over a period of 3 weeks (FIG. 2C). However, there was an unexpected particle size shrinkage between empty NE and R848 NE where R848 NE showed approximately 50 nm smaller than the empty NE in median particle diameter (R848 NE˜100 nm, empty NE˜150 nm). The reduction in particle sizes after R848 loading may result from the hydrophilic interactions between R848 (FIG. 1) and aqueous phase, which decreased the hydrophobic interactions between emulsion particles and aqueous phase.

### In Vitro Macrophage Stimulation.

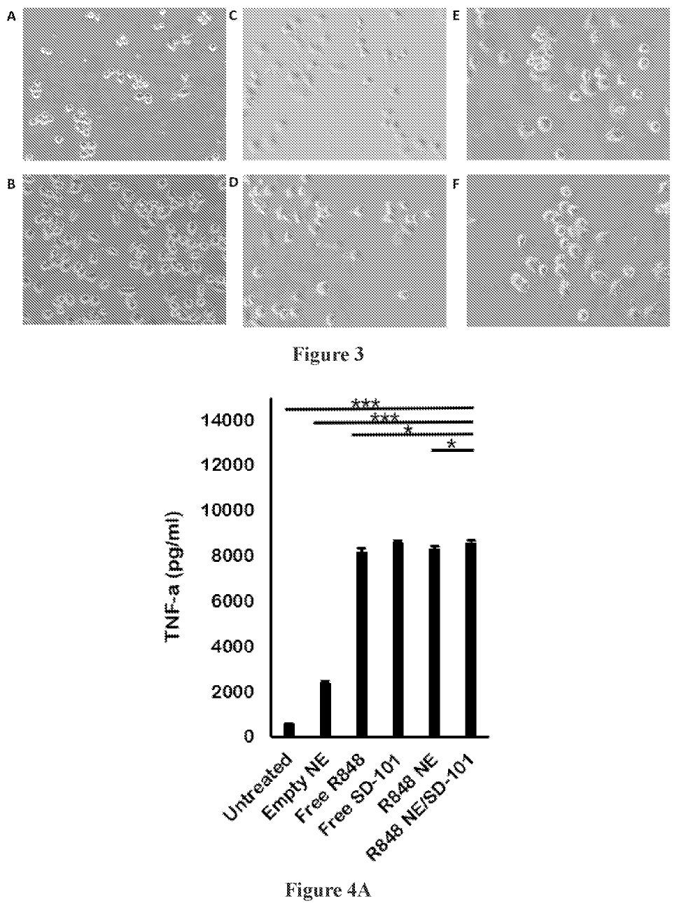

Morphological changes of macrophages upon activation can be visualized under miscroscopes, which can further be utilized to study the factors modulating pro-inflammatory (M1) and anti-inflammatory (M2) activation[33,34]. TLR7, 8, and 9 are expressed majorly on pDCs and macrophages[26,35-39], which further polarizing naïve macrophages to M1 activation[40,41]. We utilized RAW 264.7 murine macrophage cells to examine the immune stimulation carried out by R848 NE treatments as well as addition with SD-101 treatment. Our result showed that untreated RAW 264.7 generally exhibited a round form whereas empty NE-treated RAW 264.7 exhibited round form but became forming filopodia (FIG. 3, Panels A and B). In R848-treated and SD-101-treated RAW 264.7 showed partially activated macrophages with partially expansion and lamellipodia formation (FIG. 3, Panels C and D). Finally, RAW 264.7 treated with R848 NE and R848 NE/SD-101 combination showed fully activated macrophages with accelerated spreading and lamellipodia formation (FIG. 3, Panels E and F).

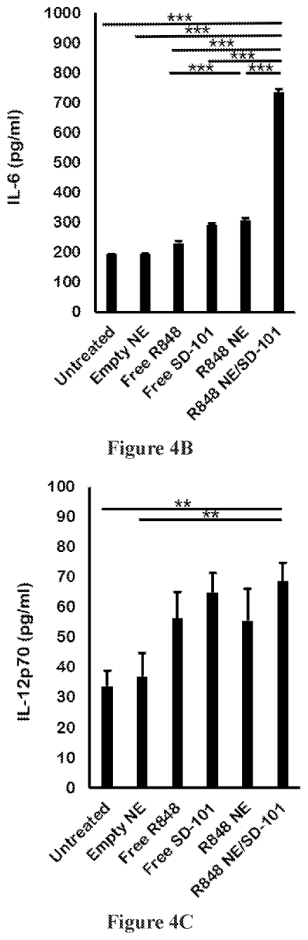

TNF-α is a pro-inflammatory cytokine which is indispensable for early immune response generation. We showed that all R848 and SD-101 treatments produced significant level of TNF-α inductions compared to the untreated group. Empty NE also induced moderate level of TNF-α due to the immune stimulation property carried out by squalene (FIG. 4A). This result corresponded with the previous research on TNFα induction by TLR7, 8 and 9 activation[42,43]. However, squalene-triggered TNF-α production was not significant compared to R848 and R848 NE treatments. In addition, treatments with SD-101 or R848 NE/SD-101 combination showed slightly higher level of TNF-α secretion compared to R848 or R848 NE treatments (FIG. 4A), suggesting that TNF-α production was favorable in TLR9 activation by SD-101 compared to TLR7/8 activation by R848.

Both TLR7/8 and TLR9 stimulate IL-6 production, which acts as both pro-inflammatory and anti-inflammatory cytokine[44-47]. Here we reported that R848 NE treatment promoted production of IL-6 compared with R848 treatment, whereas empty NE did not show significant IL-6 production (FIG. 4B). We also reported that our R848 NE and SD-101, as TLR7/8 and TLR9 agonists, respectively, synergized IL-6 production compared with individual treatments (FIG. 4B).

Lastly, IL-12p70 production biases Th1 activation resulting cellular immune responses[48,49]. There were moderate IL-12p70 production in both R848 NE and SD-101 treatment individually or in combination (FIG. 4C). We observed that squalene-based NE could potentiate IL-12p70 production stimulated by R848. In addition, the total IL-12p70 levels triggered by SD-101 and R848 NE/SD-101 combination were slightly higher than those triggered by R848 or R848 NE. In contrary to previous report on the synergistic IL-12p70 production by co-activation of duo TLRs[8,50-52], we showed that IL-12p70 production was not synergized by TLR7/8 and TLR9 co-activation (FIG. 4C).

### In Vivo Antitumor Efficacy.

Research indicated that TLR7 and TLR9 agonists generate the highest immunogenicity compared with other endosomal TLR agonists by intranasal or oral administration[53]. In addition, intratumoral combination treatment of TLR7/8 and TLR9 agonists was also demonstrated to induce the highest tumor-specific immunity compared with each agent alone[5]. However, intraperitoneal administration is easy, quick, and minimally stressful for both animal studies and patients with metastasis-stage cancer in practice. In the present study, we utilized 4 mg/kg R848 and 2 mg/kg SD-101 individually or in combination to show a synergized antitumor efficacy through intraperitoneal administration. The moderate antitumor efficacies by R848 NE and SD-101 individual treatments were observed (FIG. 5, Panels C, D. and F) compared with saline control (FIG. 5, Panel B), which correspond with the previous studies for R848 and SD-101[54,55]Although individual treatments of R848 NE or SD-101 showed significant tumor growth inhibition (50.72%±16.83% and 65.65%±12.88%), R848 NE/SD-101 combination treatment reached approximately 84.62%±28.05% total tumor growth inhibition at the end of study (Table 1) with the median tumor growth inhibition of 98.05%, suggesting the synergistic antitumor efficacy carried out by R848 NE/SD-101 combination treatment. No significant differences in body weight indicated mice treated with R848 NE and SD-101 individually or in combination had minor systemic toxicity. Among individual mouse treated with R848 NE/SD-101 combination, one mouse was observed with initial tumor size over 200 mm³ which had slightly higher tumor growth rate compared with other individuals within the same group (FIG. 5, Panel E). This suggests that additional treatments should be concorded with R848 NE/SD-101 combination strategy to eliminate large solid tumors through intraperitoneal administration.

TABLE 1
Tumor growth inhibition (TGI %) at day 10 for R848
NE or SD-101 individually or in combination.

| Treatment | Mean | Standard |
|---|---|---|
| Group | TGI % | Deviation |
| R848 NE | 50.72 | 16.83 |
| SD-101 | 65.65 | 12.88 |
| R848 | 84.62 | 28.05 |
| NE/SD-101 | | |

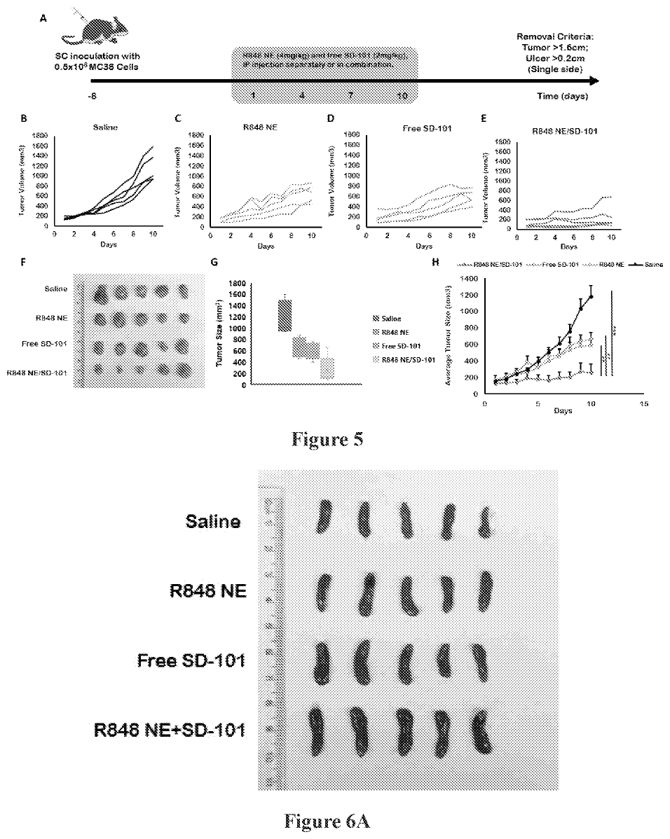

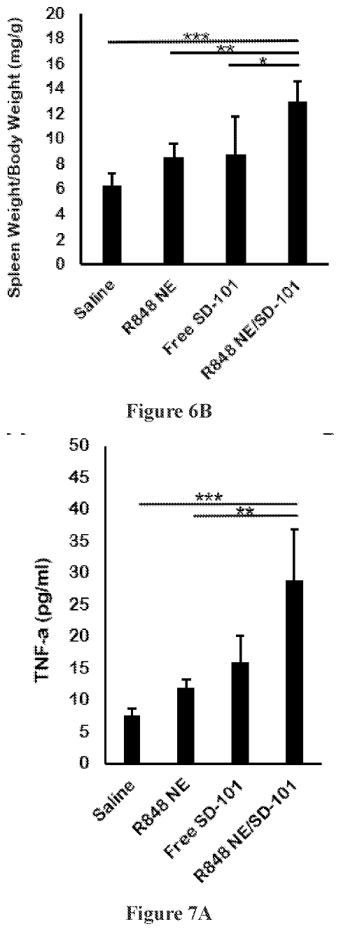

Splenomegaly indicates T cell activation and natural killer cell (NK cell) expansion[56]. Besides the significant tumor inhibition in R848 NE/SD-101 combination treatment group, we also observed a remarkable splenomegaly in R848 NE/SD-101 combination treatment group compared with individual R848 NE or SD-101-only treatment group as well as the saline control (FIGS. 6A and 6B). The remarkable splenomegaly demonstrated a synergistic antitumor immunity activation carried out by R848 NE/SD-101 combination treatment.

### In Vivo Cytokine Production.

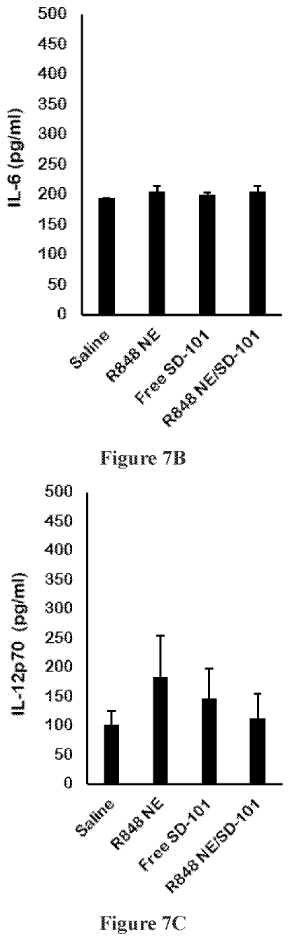

The presence of TNF-α indicates a strong pro-inflammatory cytokine release which further potentiate the tumor cell apoptosis[57]. In mouse sera, we observed a significant increase of TNF-α level among mice treated with R848 NE/SD-101 combination (FIG. 7A). This, again, demonstrated the synergistic antitumor immunity activation carried out by R848 NE/SD-101 combination treatment along with the remarkable splenomegaly and tumor inhibition. No significant change in IL-6 and IL-12p70 level was observed among mice treated with R848 NE and SD-101 individually or in combination compared with saline control (FIG. 7C), though TLR7 activation-biased IL-12p70 production was observed in mice treated with R848 NE compared with mice treated with SD-101 or R848 NE/SD-101.

### In Vivo Gene Regulation.

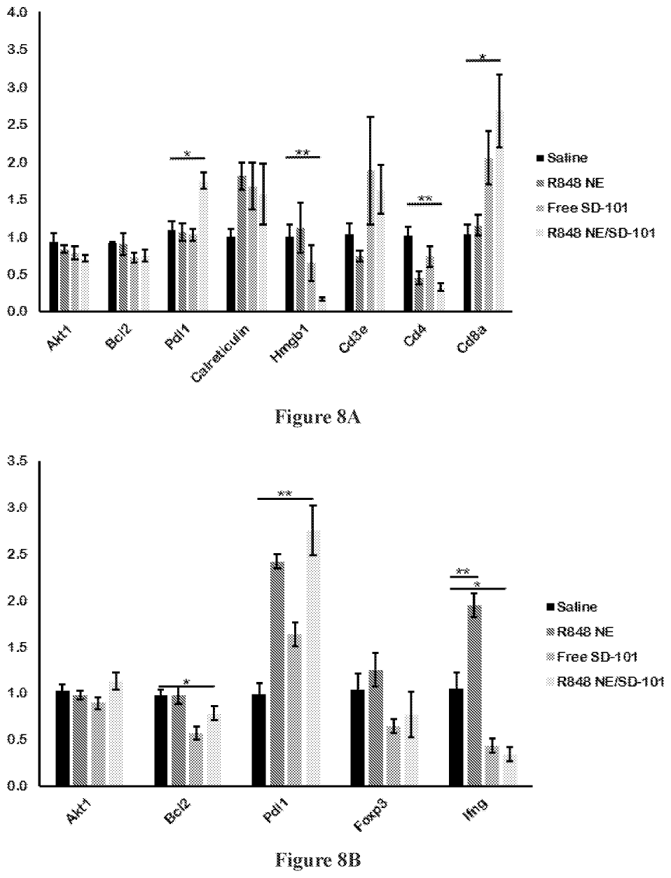

Antitumor immunity carried out by R848 NE/SD-101 combination can be attributed to cellular-mediated cytotoxicity such as CTLs or NK cells activation in concordance with immunogenic cell death (ICD) processes[58,59]. Cd8a mRNA was significantly upregulated in tumor tissues from mice treated with R848 NE/SD-101 combination along with elevated Calreticulin and Cd3e (FIG. 8A), indicating that systemic treatment with R848 NE and SD-101 synergized the antitumor immunity through CTLs and NK cells activation. Different immune regulations were observed in mice treated with R848 NE or SD-101. Foxp3, a transcription factor known as the regulatory T cell marker, was downregulated in spleens treated with SD-101 or R848 NE/SD-101 combination, suggesting that less regulatory T cells may be generated after TLR9 activation and therefore suppress the antitumor immunity by CTLs and NK cells. Cd3e and Cd4 mRNA expressions were significantly lower in tumors from mice treated with R848 NE compared with mice treated with SD-I01 (FIG. 8A), indicating less amount of CD4 T cells infiltrated into tumor microenvironment (TME) in mice treated with R848 NE and R848 NE/SD-101 combination. However, the low expression of Cd3e and Cd4 mRNA did not interfere the high expression of Cd8a mRNA in TME (FIG. 8A), potentially suggesting that CTLs and NK cells cytotoxicity directed by R848 NE or SD-I01 were T helper cell-independent in TME. Treatment with R848 NE also successfully induced Ifng expression in mice spleens whereas treatment with SD-101 suppressed Ifng expression in mice spleens (FIG. 8B). It is possible that SD-101 directed TLR9 activation increased TNF-α production (FIG. 7A) which enhanced CTLs and NK cells cytotoxicity in TME. The tumor cell lysis directed by elevated TNF-α will further trigger tumor antigen presentation and more rapidly promote immune evasion of CTLs and NK cells in TME[60,61].

Calreticulin mRNA upregulation (FIG. 8A) was observed in mice tumors treated with R848 NE and SD-I01 individually or in combination, where increase of calreticulin exposure would lead to ICD from tumor antigen phagocytosis by APCs[62]. HMGB1 has been known to negatively regulate cell proliferation where the releasement of HMGB1 induce ICD[63,64]. Hmgb1 mRNA was significantly downregulated in mice treated with SD-101 or R848 NE/SD-101 (FIG. 8A), which demonstrated that R848 NE/SD-101 treatment generates a TME that is unfavorable to cell proliferations. AKT-1, a member of AKT family, has been demonstrated to play an important role in cellular survival and to be associated with oncogenesis [65,66]. In addition, the presence of AKT-1 has also been demonstrated to concord with BCL-2 expression which functions to inhibit apoptosis and promote tumor proliferation[67-69]. ICD induced by R848 NE and SD-101 alone or in combination also showed slightly Akt1 and Bcl2 mRNA downregulation (FIG. 8A), also indicating a proliferation unfavorable TME.

TLR7/8 and TLR9 activation in cancer cells, lymphocytes, and pDCs are also associated with PD-L1 upregulation, which is an immune checkpoint for tumor immune escape[70-73]. Mice treated with R848 NE/SD-101 combination exhibited the highest level of Pdl1 mRNA in both spleen and tumor tissue compared with mice treated with individual treatments or saline control (FIGS. 8A and 8B). Although people commonly accept the concept of immune escape established by the binding between PD-L1 on cancer cells and PD-1 on T cells[74], more research now indicate that cancer cells can express both PD-1 and PD-L1, and PD-L1 is also found on certain immune cells such as macrophages and dendritic cells[75-77]. Considering the role of PD-L 1 on antigen-presenting cells that may inhibit T cell function[77], we suggest that the R848 NE/SD-101 combination treatment through intraperitoneal injection could further exhibit higher antitumor efficacy by incorporating anti-PD-L1 therapeutics to eliminate large tumors or metastatic tumors

### Conclusions

Our study presented a R848-loaded squalene-based nanoemulsion formulation that had better encapsulation efficiency compared with reported liposomal formulations. We also showed that R848 NE is highly stable during long-term storage at 4° C. Elevated TNF-a level in vitro demonstrated the strong immune activation by R848 NE and SD-101 individually. Brightfield images and cytokine levels in vitro and in vivo further illustrated a synergistic immune activation when R848 NE and SD-101 were treated in combination. Ultimately, we demonstrated that R848 NE/SD-101 combination treatment strongly enhanced the antitumor efficacy in vivo by greatly suppressed tumor growth over 80%. Our work indicated that intraperitoneal administration of R848 NE and SD-101 in combination could be a strong approach for CTLs and NK cells-dependent systemic antitumor treatment. Furthermore, the synergized PD-L1 upregulation in vivo by R848 NE/SD-101 combination implied greater antitumor potential while incorporating R848 NE/SD-101 treatment with anti-PD-L 1 therapeutics.

### Example 2: pH-Sensitive Lipid Nanoemulsion (PSNE) Formulation as an Effective Delivery System to Deliver CpG Oligonucleotides and Therapeutic Oligonucleotides for Anticancer Therapeutics

### Overview

Cancer vaccines are designed to trigger a specific and long-lasting immune response against tumor antigens. A well-designed vaccine should trigger dendritic cell (DC) activation by defined tumor antigens and include adjuvants that stimulate the expansion of naïve T cell repertoire into effector T cells. It has been shown that a combination of toll-like receptor (TLR) agonists with tumor-associated antigens (TAAs) triggers DC maturation, cytotoxic T cell (CTL) activation, and tumor regression. However, TLR activation induces PD-L1 expression on monocytes and dendritic cells, and even cancer cells, which protect them from CTL attack. The elevation of PD-L 1 on cancer cells or monocytes can lead to CTL exhaustion and dysfunction. Here, we hypothesize that co-delivery of TLR agonists and anti-PD-L1 (LNA ASO/siRNA) leads to a higher level of dendritic cell activation and maturation, which induces potent T cell activation and anti-tumor activities. In addition, by co-delivering TLR agonists with anti-PDL1 ASO, the overall immuno-suppressive environment could be skewed back to immuno-permissive conditions, which is associated with regulatory T cell population reduction and macrophage repolarization. A novel pH-sensitive lipid nanoemulsion formulation, or PSNE, was developed to co-deliver CpG oligonucleotide-based TLR agonists as well as anti-PDL1 LNA ASO in a single particle. The PSNE lipid nanoparticles had particle sizes of ˜85-95 nm, which was ideal for delivery into the tumor microenvironment (TME) through enhanced permeation and retention (EPR) effect. In an MC-38 syngeneic murine tumor model, single agents of either oligonucleotide-based TLR agonist (class-C CpG) or anti-PDL1 LNA ASO in PSNE could lead to ˜45% of tumor growth inhibition (TGI), whereas co-encapsulation of both CpG and ASO into a single particle lead to a higher TGI at ˜73%. Flow cytometry analysis showed that inhibitory regulatory T lymphocytes (Treg) population frequency in the spleen was reduced in single-agent groups and further reduced in the combination group, indicating the reversal of immune-suppressive environment. Furthermore, PD-L1 expression was assessed on splenic macrophages, T lymphocytes as well as overall splenocytes by flow cytometry and RT-qPCR. The results showed that PD-L1 expression was downregulated in macrophages in all treatment groups, but not in splenic T lymphocytes. In addition, PD-L 1 mRNA levels were reduced by ˜60% in groups treated with anti-PDL1 ASO, in both single-agent and combination groups, illustrating that the PSNE lipid nanoparticles were taken up by phagocytes and overcame PD-L1 upregulation by TLR activation.

In summary, we demonstrated that co-encapsulation of CpG-oligonucleotide-based TLR agonist and anti-PDL1 ASO in the PSNE formulation enhanced the delivery of both agents to the tumor and the spleen and was more effective in activating the immune system against the tumor.

### Materials and Methods

Squalene was purchased from Sigma-Aldrich (St. Louis, MO). DOPC and DOPE were purchased from Avanti Polar Lipids (Alabaster, AL). DODMA and DMG-PEG₂₀₀₀ were purchased from NOF America (White Plains, NY). Any chemicals or buffers unless otherwise stated were purchased from Fisher Scientific (Hampton, NH).

DOPC, DOPE, Squalene, DODMA and DMG-PEG2000 were dissolved in ethanol as a mixture at a molar ratio of 15:28:10:45:2. Next, the lipid ethanol solution was rapidly injected into an acidic buffer to form empty pH-sensitive nanoemulsions (PSNEs) at a lipid concentration of 8 mg/mL. In the meantime, nucleic acid cargos, including SD-101 and anti-PDL1 locked nucleic acid (LNA) antisense oligonucleotides (ASO), were dissolved in nuclease-free water at 0.4 mg/mL. Empty PSNEs and nucleic acid cargos were warmed up to 60° C. prior to the mixing. Then, the cargo was added dropwise to the empty PSNEs on slow vortex at a weight ratio of 1 to 20 until nucleic acid-encapsulated PSNEs were formed. The products were incubated at 37° C. for 10 minutes and stored at 4° C. before use. The particle sizes and zeta potential (ζ) of nucleic acid-loaded PSNEs were analyzed by dynamic light scattering on a NICOMP Z3000 Nano DLS/ZLS System (Entegris, Billerica, MA).

### Animal Models.

C57BL/6 mice were purchased from Charles River Laboratory. Animals were housed in a temperature-controlled room under a 12 hr light/12 hr dark cycle and fed normal chow diet. All animal studies were reviewed and approved by the Ohio State University Institutional Laboratory Animal Care and Use Committee. Both male and female mice were used for experiments.

MC-38 murine colorectal cancer cell line was a kind gift obtained from Dr. Christopher Coss at The Ohio State University College of Pharmacy. MC-38 syngeneic subcutaneous murine colorectal cancer model was developed by injecting 1 million MC-38 cells suspended in PBS into the right flank of each mouse subcutaneously. Tumor sizes and body weights were monitored daily using a digital caliper and a lab scale, respectively.

The treatments were started when the average tumor sizes reached 80 mm³. Mice were randomized into 6 groups of 5 mice per group. The treatments, including normal saline control, SD-101 oligonucleotides, SD-101 in PSNE, anti-PDL1 LNA in PSNE, SD-101 and anti-PDL1 LNA in PSNE, and Resiquimod and SD-101 in PSNE, were given once every three days with a total of 5 doses. Mice were sacrificed 6 hours after the final dose and organs as well as whole blood samples were collected for further analysis.

Serum samples were obtained from whole blood samples by incubating blood at RT for 30 minutes, followed by 2,000×g centrifugation for 20 minutes. The samples were stored at −80° C. prior to analysis. Cytokine concentrations were measured by uncoated ELISA kits purchased from Invitrogen per manufacturer's protocols.

Single cell splenocyte suspensions were harvested from spleens by the following procedures. Spleens were gently meshed through 70 um nylon meshes (Thermo Scientific) using a sterile 5-mL syringe plunge and washed with cold RPMI medium twice into six-well plates. Crude splenocyte suspensions were then centrifuged at 500×g for 5 minutes at 4° C. to obtain cell pellets. Cell pellets were resuspended in 1×RBC lysis buffer to lyse red blood cells for 5 minutes at RT and 10-fold volume of PBS was added to stop the reaction. After 500×g centrifugation for 5 minutes, the cell pellets were then resuspended in FACS staining buffer to form single cell splenocyte suspensions.

Antibodies were purchased from Biolegend (San Diego, CA), and surface cell staining was done per manufacturer's protocol. Intracellular staining was done by using Biolegend True-Nuclear™ Transcription Factor Buffer Set per manufacture protocol. The stained cells were analyzed on a BD LSRForetessa Flow Cytometer at the Flow Cytometry Shared Resources at The Ohio State University Comprehensive Cancer Center.

Splenocyte messenger RNA for gene expression analysis was extracted from splenocyte suspensions using TRI reagent per manufacturer's protocol. cDNA was prepared using High-Capacity cDNA Reverse Transcription Kit (Invitrogen). Real-time PCR was conducted on a QuantStudio 7 Flex Real-Time PCR System. The relative amount of RNA level was calculated and compared according to the 2^{-ΔΔCt} method.

### Results and Discussion

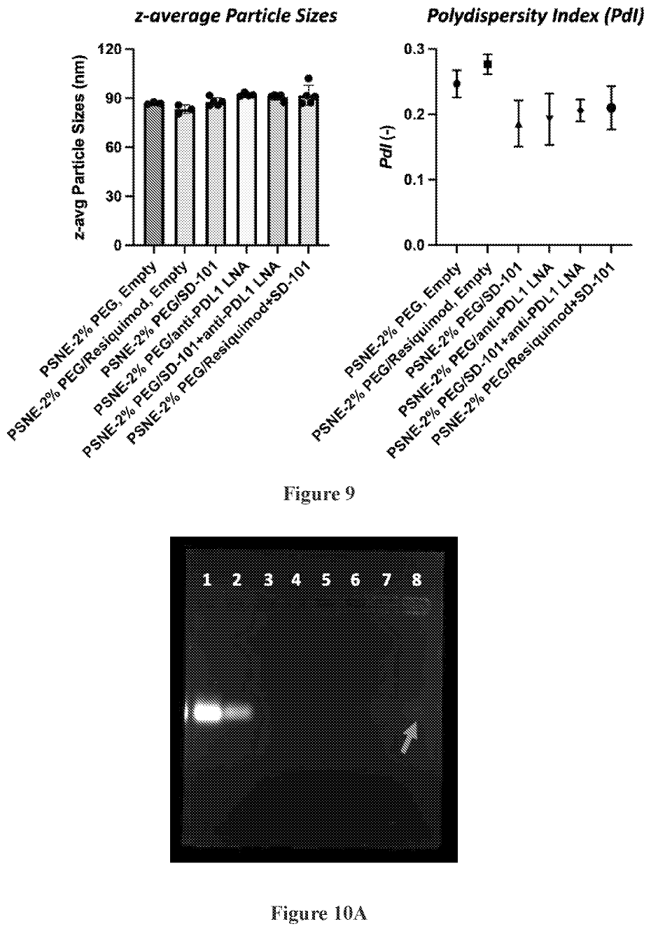

PSNEs encapsulating oligonucleotides and small molecules were successfully formulated with high colloidal stability. Particle sizes ˜80-100 nm were obtained for both empty PSNEs and PSNEs loaded with either SD-101 CpG ODNs or anti-PDL1 LNA ASOs. The polydispersity indexes (PdI) were ˜0.15-0.25, indicating narrowly distributed particle sizes were obtained in all samples (FIG. 9). Empty PSNEs had a mean zeta potential at +8.06 mV in 20 mM PB at pH 4, and encapsulated PSNEs had mean zeta potentials around neutral in 10 mM phosphate buffer at pH 7.

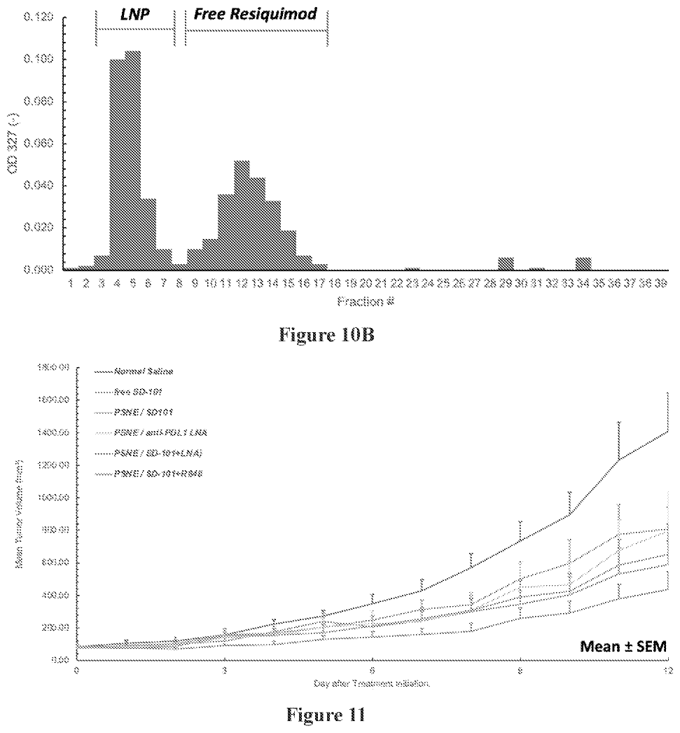

Next, the encapsulations of ODN and small hydrophobic molecule cargos in PSNEs were analyzed by gel retardation assay and size exclusion chromatography, respectively. The gel image showed that ODNs were highly encapsulated in PSNEs, with a small amount of SD-101 adsorbed onto the PSNE surface while Resiquimod was added to the formulation. The percentage encapsulation of Resiquimod in PSNEs was ˜53.8% by calculating the ratios of two peaks on the chromatogram obtained by OD327 using a NanoDrop spectrophotometer (FIGS. 10A-10B).

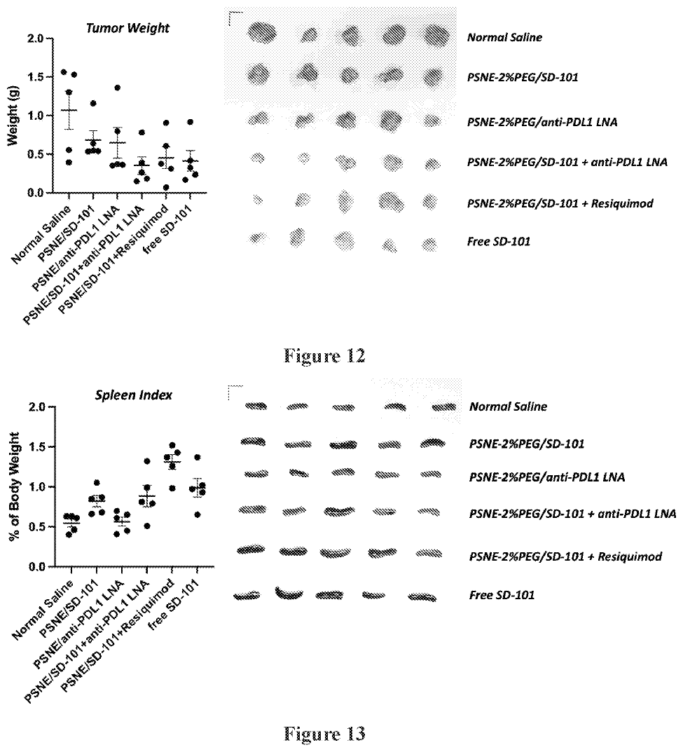

The combination of SD-101 CpG ODNs and anti-PDL1 LNA ASOs in PSNEs showed a good antitumor effect in MC-38 murine syngeneic colon cancer model with a mean tumor growth inhibition (TGI) ˜73%. Two groups of mice treated with single agent in PSNE (either SD-101 ODNs or anti-PDL1 LNA ASOs) both showed TGIs of ˜46%, while mice treated with PSNE encapsulating SD-101 and Resiquimod had a mean TGI of 57% (FIG. 11). The tumor weights were recorded and were represented the actual tumor sizes after the treatments. The results showed the similar trend with the tumor volumes, as the PSNE combination group showed significant in tumor growth inhibition compared to normal saline control using one-way ANOVA (FIG. 12).

The spleen index (spleen weight presented in the percentage of body weight, FIG. 13) result indicated that PSNEs had similar immune system activation effects as SD-101 oligonucleotides. Also, dual TLR agonists in combinations further activated the immune system (by expanding immune cell populations). Besides, the results also indicated that PSNEs encapsulating anti-PDL1 LNA ASOs did not have immune system activation as monotherapy. The tumor growth inhibition of PSNE/anti-PDL1 LNA ASOs is likely a result of PD-L1 downregulations in tumor sites, either from LNA delivery to cancer cells or TME immune cells.

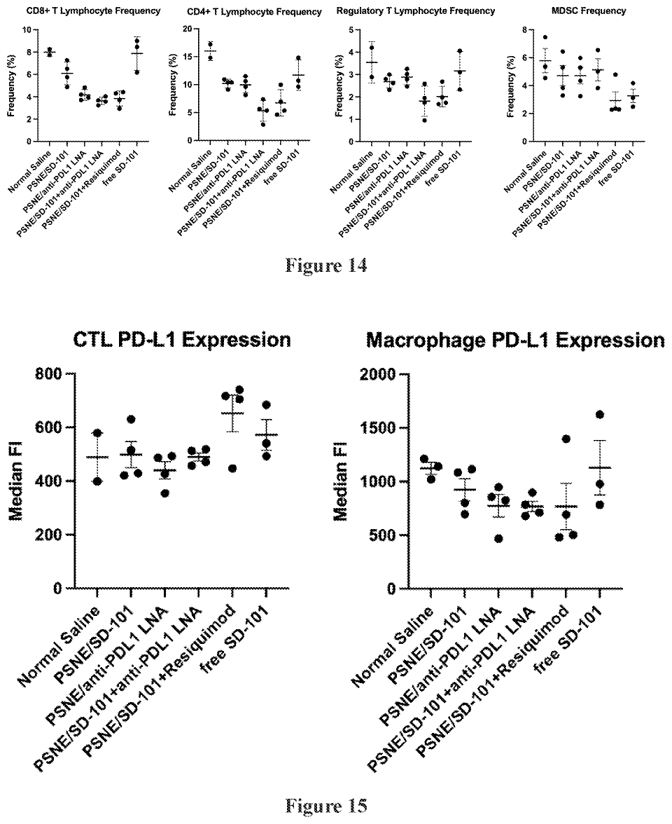

In a flow cytometry analysis (FIG. 14), all PNSE-based treatment groups had significant decreases in T lymphocyte population frequencies, either CD4+ T helper cells, CD8+ cytotoxic T cells or regulatory T cells, compared to the normal saline control group. The results could be interpreted as T cell activation followed by infiltration to TME. IHC staining T cell populations can be done to confirm the hypothesis. MDSC frequencies had no significant changes after all treatments in MC-38 tumor model.

Surface PD-L1 markers on both lymphocytes and myeloid cells were examined by flow cytometry to investigate the PD-L1 protein expressions on immune cells after treatments (FIG. 15). The results showed that surface PD-L1 protein levels on macrophages were downregulated in groups treated with anti-PDL1 LNA ASOs, given that macrophages had the ability to phagocytose PSNEs and anti-PDL1 LNA can induce mRNA downregulation. Nonetheless, PD-L1 protein expressions on cytotoxic T lymphocytes did not have significant downregulations after treatment with anti-PDL1 LNAs, suggesting relatively low transfection of T cells. As we expected, PD-L1 protein expression level in TLR (SD-101 ODNs and Resiquimod) treatment groups were upregulated due to NFκB-MyD88 activation pathway. This also indicated that anti-PDL1 LNA was able to overcome the PD-L1 upregulation by TLR agonists.

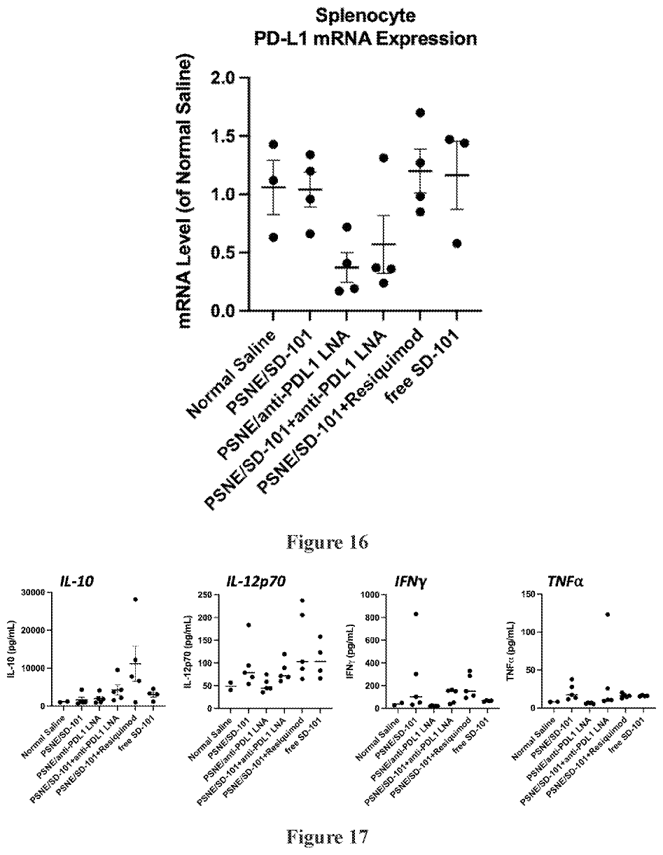

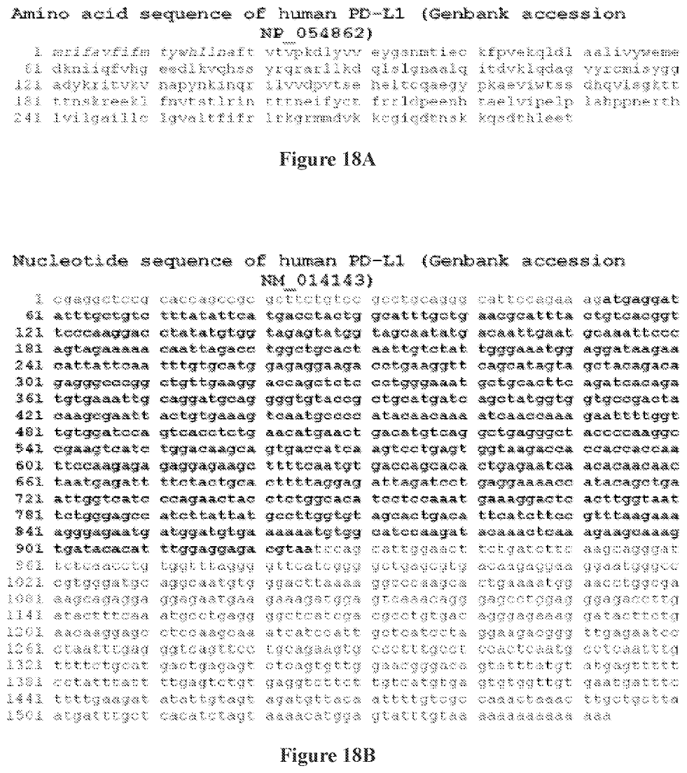

By doing splenocyte mRNA analysis, we assessed the cytokine secretion profile as well as overall PD-L1 levels after treating with TLR agonists and/or anti-PDL1 LNA. The results demonstrated an excellent activity for LNA in downregulating PD-L1 mRNA among splenocytes (FIG. 16).

Based on the cytokine ELISA results (FIG. 17), it was clear that mice treated with PSNE/anti-PDL1 LNA were not in an inflammatory state (basal levels of proinflammatory cytokines TNFα and IFNγ); therefore, the tumor growth inhibition was related to the anti-PDL1 LNA ASO delivery to the tumors. Also, the results also showed that TLR agonists were able to prime the immune system toward pro-inflammatory stage by elevating [L-12 levels.

### Conclusion

In conclusion, this study showed that PSNEs were effective delivery vehicles of anti-PD-L1 LNA ASOs for antitumor therapy and was able to co-deliver ASOs with resiquimod. It showed that Anti-PD-L1 acted synergistically with TLR activation, therefore, the combination of anti-PD-L I oligo (also siRNA) therapy and TLR agonists are highly synergistic. It was further shown that PSNE-delivered resiquimod was very effective, and was synergistic with CpG ODN SD-101, providing a novel approach to delivery TLR combinations for therapeutic purposes. Triple combination of dual TLR agonists and anti-PD-L1 produced the best overall therapeutic response.

### Example 3: Nanoemulsion-Based Lipid Nanoparticles as a Delivery Platform for Toll-Like Receptor Agonist-Based Immunotherapeutics Against Cancers

Traditional cancer therapies, including surgery, radiation therapy, and chemotherapeutics have been practiced for decades to abolish tumor tissues, but those treatments have significant side effects. To reduce side effects, next-generation medications, including targeted therapy and immunotherapy, came into clinics after the first-approved monoclonal antibody rituximab in 1997. Compared to the targeted therapy targeting cancer cell itself, immunotherapy opens a new battlefield by targeting the immune system. Well-known immunotherapies, including immune checkpoint blockade (ICB), chimeric antigen receptor (CAR) T cell therapy, oncolytic viruses, and cancer vaccines have drawn increasing attention in treating cancer patients.

Cancer vaccines establish a promising strategy to trigger a specific and long-lasting immune response against tumor antigens. A well-designed cancer vaccine should trigger dendritic cell (DC) activation by defined tumor antigens and proper adjuvants, which stimulates the enlargement of naïve T cell repertoire into effector T cells. Many research groups have shown that combining toll-like receptor (TLR) agonists with tumor-associated antigens (TAAs) could initiate DC maturation, cytotoxic T cell (CTL) activation, and tumor shrinkage.

Tumor progression can be described by immunosurveillance with three stages: elimination of cancer cells by the immune system, cancer equilibrium, and tumor escape. The tumor escape evolves in genetic alternations of tumor antigens that avoid immune recognition as well as the tumor-extrinsic mechanisms with active immune suppression. Low expression level of major histocompatibility complex (MHC) class I on cancer cell surface reduces the recognition and eradication of CD8⁺ T cells. The tumor also produces immunosuppressive cytokines such as transforming growth factor β (TGF-β) or soluble Fas ligand to mediate regulatory T cells (T_{reg}s) response on suppressing antitumor effector T cells. To overcome the immunosuppressive barrier built by TME or reduced-function CTL, immunomodulating agents are introduced to the clinics. Immune checkpoint blockade (ICB) agents can help to resume CTL functions by inhibiting negative regulator signals on the T cell surface. Typical targets are cytotoxic T-lymphocyte-associated antigen 4 (CTLA-4) or programmed cell death 1 (PD-1). Blocking the CTLA-4 or the PD-1 with monoclonal antibodies showed enhancement in effector T cell activation and proliferation.

An alternative pathway to upregulate immune system is to activate T cell functions by stimulating dendritic cells. Dendritic cells are the most potent and efficient antigen-presenting cells (APCs) of the innate immune system that can present exogenous antigens to CD4⁺ T cells and CD8⁺ T cells through MHC class II and I, respectively. DC cell maturation and activation are initiated through the antigen uptake and recognition by pattern recognition receptors (PRRs) such as toll-like receptors (TLRs). Matured DCs migrate to draining lymph nodes with high expression of MHCs and antigen-presenting ability. In draining lymph nodes, DCs meet with naïve T cells and instigate T cell activation. T cell activation requires three signals: antigen-associated MHC interaction with T cell receptor (TCR), co-stimulatory ligand (CD80, CD86, CD40) recognition and pro-inflammatory cytokines. All those signals facilitate CD4⁺ naïve T cells to differentiate into Tₕ₁ cells and enhance the cytotoxic response of CTLs. Therefore, the basic principle behind DC vaccines entails the incubation of DCs with tumor antigens/stimulator cocktail to produce DC maturation and the boost of T cell activation after injecting into patients. However, the objective response rate (ORRs) to DC vaccines in cancer patients rarely passed 15%.

To enhance the DC maturation, antigen uptake, and T cell activation, TLR agonists are introduced as stand-alone antitumor agents or as adjuvants combined with TAAs. TLR signal transduction cascades induce the production of type-1 interferons and inflammatory modulators, which facilitate the further T lymphocyte activities. Previously published research suggested injecting either free-form CpG ODNs or CpG-ODN nanoparticles with tumor antigens showed potent antitumor response in vivo. Besides CpG ODNs, synthetic TLR3 agonist poly(I:C) was used in several studies to show the facilitated induction of DC maturation and T cell-related antitumor activity.

CpG oligonucleotides are well-known for their TLR9 activation ability with type-1 interferon production, triggering the downstream processes of both innate and adaptive immunity activation. Many studies have been done using different subclasses of CpG ODNs as vaccine adjuvants to stimulate dendritic cell maturation and initiate T-cell (either Tₕ₁ or Th2) mediated immunity. In a research study, the researchers indicated that applying CpG ODN 2006 (a class-B CpG ODN) could increase the infiltrated T cells and activated DCs within draining lymph nodes, suggesting CpG ODN 2006 has the ability to stimulate DC activation as a vaccine adjuvant. Also, administering cell lysate together as the antigen source could facilitate not only the DC maturation process but also CTL generation. Besides, they demonstrated that applying CpG ODN 2006/lysate combination could significantly prolong the tumor-burden mice survival, compared to CpG ODN 2006 single treatment, indicating the supporting role of CpG ODNs in the vaccine construct, working as adjuvants.

CpG ODNs are ligands for TLR9, which is primarily expressed in human plasmacytoid dendritic cells specializing in secreting type-1 interferons. However, different subclasses of CpG ODNs have distinct abilities in stimulating type-1 interferon production due to the oligonucleotide structures. Class-A CpG ODNs include poly-G motifs at both 5′ and 3′ ends and a self-complementary palindrome containing one or more CpG motifs. Class-A CpG ODNs are strong IFN-α inducer. On the other hand, class-B CpG ODNs have complete phosphorothioate backbones but without high level structures, giving weak ability to induce IFN-α production but strong ability to stimulate B cell TLR9. It is reported that class-A CpG is more active in supporting natural killer cells (NK cells) and CTL function and granzyme-B content in CTLs, which is mediated mainly by type-1 interferons. Besides, IFN-α is an essential cytokine in activating NK cells and in partial activation and proliferation of memory CD8⁺ T cells. Interferon-y (IFN-γ) secreted by activated NK cells also triggers Tₕ₁ cell-mediated immunity in combination with IL-12 secreted by activated DCs. As a result, applying class-A CpG ODNs instead of class-B CpG ODNs should initiate substantial innate and adaptive immunity as well as pro-inflammatory Tₕ₁ and CTL response.

In this example, we develop a series of highly fusogenic nanoemulsion-based lipid nanoparticle constructs to co-encapsulate negative-charged ODNs with different immune system booster factors, such as anti-PDL1 antisense oligonucleotides or tumor antigen peptides, at high loading amount as well as using a TLR agonist cocktail (CpG ODNs, poly(I:C), or Resiquimod) as the vaccine adjuvant to react on different DC subpopulations for the antigen-presenting enhancement. We also evaluated a TLR agonist combination to demonstrate the synergism over single agonist. The co-encapsulated TLR agonists give higher pro-inflammatory cytokine secretion and more intensive T cell activation. These strategies have the potential to overcome the current barrier of low stimulating effect on the immune system in vivo due to the delivery deficiency to APCs.

### Materials and Methods

### Materials.

Squalene, polyinosinic:polycytidylic acid (polyI:C), monophosphoryl lipid A (MPLA) and D-α-tocopheryl polyethylene glycol 1000 succinate (TPGS) were purchased from Sigma-Aldrich (St. Louis, MO, USA). DSPC, DOPC, DOPE, and cholesterol were purchased from Avanti Polar Lipids (Alabaster, AL, USA). DODMA and DMG-mPEG₂₀₀₀ were purchased from NOF America (White Plains, NY, USA). DLin-MC3-DMA was purchased from DC Chemical (Shanghai, China). Resiquimod (R848), imiquimod (R837), and amphotericin B were purchased from MedChemExpress (Monmouth Junction, NJ, USA). Tocopherol succinate (TS) and didodecyldimethylammonium bromide (DDAB) were purchased from TCI America (Tokyo, JP). SD-101 CpG oligodeoxynucleotides (SD-101), CpG 2216 oligodeoxynucleotides, and 2′-OMethyl-modified murine anti-PDL1 antisense oligodeoxynucleotide gapmer (2′-OMe anti-PDL1 ASO) were synthesized by Alpha DNA (Montreal, CA). Locked nucleic acid-modified murine anti-PDL1 antisense oligodeoxynucleotide gapmer (anti-PDL1 LNA ASO) was synthesized by Integrated DNA Technologies (IDT, Coralville, IA, USA). Any chemicals or buffers otherwise stated were purchased from Fisher Scientific (Hampton, NH, USA).

### Preparation of Cationic Nanoemulsion Incorporating TLR Agonists.

For CpG 2216 and poly(I:C) cationic nanoemulsion, squalene, DDAB and TPGS were mixed into a lipid ethanol mixture at a molar ratio of 50:35:15. Next, the lipid-ethanol solution was rapidly injected into 20 mM HEPES buffer at pH 7.4, forming empty cationic nanoemulsions at a lipid concentration of 25.Omg/mL. To produce oligonucleotide-encapsulated cationic nanoemlusions, CpG 2216 or poly(I:C) oligonucleotide solution (in DEPC water) at 2.Omg/mL was added dropwise to the empty lipid nanoemulsions on slow vortex at a weight ratio of 1 to 12.5 until nucleic acid-encapsulated cationic nanoemulsions were formed. For imiquimod nanoemulsions, squalene, TS and TPGS were mixed into a lipid-ethanol mixture at a molar ratio of 75:15:10. Imiquimod (in DMSO) was added to the lipid-ethanol mixture to form homogeneous solution prior to ethanol diffusion. Next, the lipid-ethanol solution was rapidly injected into 20 mM Acetate buffer at pH 4.0, forming imiquimod-incorporated nanoemulsions at a lipid concentration of 30.Omg/mL and an imiquimod concentration of 1.5 mg/mL. The particle sizes and zeta potential of nucleic acid-loaded pH-sensitive nanoemulsion were analyzed by dynamic light scattering with a NICOMP Z3000 Nano DLS/ZLS Systems (Entegris, Billerica, MA).

### Preparation of Cationic Nanoemulsion Incorporating Tumor Antigen Peptides and TLR Agonists.

Squalene, DOTAP and TPGS were mixed into a lipid ethanol mixture at a molar ratio of 68:30:2. Next, the synthetic peptide P1A (LPYLGWLVF, SEQ ID NO:3) dissolved in DMSO, Resiquimod or MPLA was added to the lipid mixture to form homogeneous solution followed by the rapid injection of lipid/peptide/TLRa mixture into 20 mM phosphate buffer, pH 7.4 with 10% sucrose, to form peptide/TLRa loaded cationic nanoemulsions at a lipid concentration of 10.Omg/mL. To form oligonucleotide-encapsulated cationic nanoemulsions, the SD-101 oligonucleotide solution (in DEPC water) was added dropwise to the empty lipid nanoparticles on slow vortex at a weight ratio of 1 to 10 until nucleic acid-encapsulated cationic nanoemulsions were formed. The final concentrations of P1A peptides, MPLA, Resiquimod, and SD-101 were 1.0, 0.2, 0.2, and 0.2 mg/mL, respectively (or according to the formulation table). The control for each group was performed at the same formulation but without P1A peptides. The products were stored in −20° C. before use. The particle sizes and zeta potential of nucleic acid-loaded pH-sensitive nanoemulsion were analyzed by dynamic light scattering with a NICOMP Z3000 Nano DLS/ZLS Systems.

### Preparation of Standard Lipid Nanoparticles Incorporating CpG Oligonucleotides and Anti-PDL1 LNA Gapmers.

DSPC, cholesterol, DLin-MC3-DMA and DMG-mPEG₂₀₀₀ were mixed into a lipid ethanol mixture at a molar ratio of 10:38.5:50:1.5. Next, the lipid ethanol mixture was rapidly injected into an acidic phosphate buffer to form empty lipid nanoparticles at a lipid concentration of 10.0 mg/mL. To form oligonucleotide-encapsulated lipid nanoparticles, the oligonucleotide solution (in DEPC water) was added dropwise to the empty lipid nanoparticles on slow vortex at a weight ratio of 1 to 10 until nucleic acid-encapsulated lipid nanoparticles were formed. The products were incubated at 37° C. for 10 minutes and stored in 4° C. before use. The particle sizes and zeta potential of nucleic acid-loaded pH-sensitive nanoemulsions were analyzed by dynamic light scattering with a NICOMP Z3000 Nano DLS/ZLS Systems.

Preparation of pH-Sensitive Nanoemulsion (PSNE) Lipid Nanoparticles.

DOPC, DOPE, Squalene, DODMA and DMG-mPEG₂₀₀₀ were mixed into a lipid-ethanol mixture at a molar ratio of 15:28:10:45:2. Next, the lipid ethanol mixture was rapidly injected into an acidic phosphate buffer to form empty pH-sensitive nanoemulsion at a lipid concentration of 8.Omg/mL. In the meanwhile, nucleic acid cargos, including SD-101 and anti-PDL1 lucked nucleic acid (LNA) antisense oligonucleotides (ASO), were dissolved in DEPC water at 0.4 mg/mL. Empty pH-sensitive nanoemulsions and nucleic acid cargos were heated up to 60° C. prior to the mixing. Then, the cargo was added dropwise to the empty pH-sensitive nanoemulsion on slow vortex at a weight ratio of 1 to 20 until nucleic acid-encapsulated lipid nanoparticles were formed. The products were incubated at 37° C. for 10 minutes and stored in 4° C. before use. The particle sizes and zeta potential of nucleic acid-loaded pH-sensitive nanoemulsion were analyzed by dynamic light scattering with a NICOMP Z3000 Nano DLS/ZLS Systems.

Preparation of Next Generation pH-sensitive Nanoemulsion (PSNE-Chol) Lipid Nanoparticles.

DOPE, cholesterol, squalene, DLin-MC3-DMA and DMG-mPEG₂₀₀₀ were mixed into a lipid-ethanol mixture at a molar ratio of 7:38:5:48:2. Next, the lipid-ethanol mixture was rapidly injected into an acidic phosphate buffer/tris buffer to form empty pH-sensitive nanoemulsions at a lipid concentration of 8.Omg/mL. In the meanwhile, nucleic acid cargos, including SD-101, anti-PDL1 lucked nucleic acid (LNA) antisense oligonucleotides (ASO) or the mixture of both, were dissolved in DEPC water at 0.4 mg/mL. Empty pH-sensitive nanoemulsions and nucleic acid cargos were heated up to 60° C. prior to the mixing. Then, the cargo was added dropwise to the empty pH-sensitive nanoemulsions on quick vortex at a weight ratio of 1 to 20 until nucleic acid-encapsulated lipid nanoparticles were formed. The products were titrated to pH 7 by 0.1N NaOH, incubated at 37° C. for 10 minutes, and stored at 4° C. before use. The particle sizes and zeta potential of nucleic acid-loaded pH-sensitive nanoemulsions were analyzed by dynamic light scattering with a NICOMP Z3000 Nano DLS/ZLS.

### Cell Culture.

Hepa1-6, MC-38 and RAW264.7 were kind gifts obtained from Drs. Kalpana Ghoshal at The Ohio State University College of Medicine, Christopher Coss and Peixuan Guo at The Ohio State University College of Pharmacy, respectively, and were cultured in DMEM (Millipore Sigma) supplemented with 10% FBS (Millipore Sigma) and antibiotics-antimycotics (Invitrogen) under 37° C. humidified atmosphere with 5% CO₂.

### In Vitro Gene Regulation Evaluation by RT-qPCR.

Cells were seeded at a density of 0.25-0.5 million per well in 6-well plates 24 hours prior to the LNP treatments. Next, cells were treated with the LNPs in complete media and were incubated for 24 hours before harvest. Total RNA in the cells was extracted using TRI reagent (Zymo Research) per manufacturer's protocol. cDNA was prepared by High-Capacity cDNA Reverse Transcription Kit (Invitrogen). Real-time PCR was conducted on a QuantStudio™ 7 Flex Real-Time PCR System using SsoAdvanced® Universal SYBR Green Supermix (Bio-Rad). The relative amount of RNA level was calculated and compared according to the 2^{-ΔΔCt} method.

### In Vitro Surface Protein Expression Evaluation by Flow Cytometry.

Cells were seeded at a density of 0.5-0.7 million cells per well in 60 mm cell culture dishes 24 hours prior to the LNP treatments. Next, cells were treated with the LNPs, murine interferon gamma, or LPS at various concentrations in complete media. Cells were washed with PBS twice followed by harvested using enzyme free cell dissociation solution, hank's based (Millipore Sigma). Cell suspensions were spun down at 500×g at 4° C. and the pellets were washed with PBS once followed by the resuspension in FACS staining buffer. Single cell suspensions were fixed in 1% PFA in PBS for 45 minutes at room temperature before stained with two biomarkers: mCd86-BV650 and mCD274-PE (Biolegend, San Diego, CA, USA) per manufacturer's protocol. The stained cells were analyzed on a BD LSRForetessa Flow Cytometer in the Flow Cytometry Shared Resource Core at The Ohio State University Comprehensive Cancer Center. Data were analyzed in FlowJo.

### Cytotoxicity Under Macrophage Condition Media.

RAW264.7 cells were seeded at a density of 0.5 million per well in 6-wells plates 24 hours prior to the LNP treatments. Next, RAW cells were treated with next generation PSNE (PSNE-Chol) LNPs incorporating SD-101 CpG ODN or anti-PDL1 LNA, LPS, or combinations. Cultured condition media were harvested, spun down to remove cell debris, and stored at −80° C. before use. MC-38 and Hepa1-6 were seeded at a density of 3000-5000 cells per well in 96-well plates 24 hours prior to the condition media treatments. Cells were treated with 100 μL of the condition media in quadruplets and were incubated for 72 hours. The cell viability was examined by CellTiter Glo (Promega) utilizing a Biotek Synergy Hi Plate Reader.

### Mice Study and MC-38/Hepa1-6 Syngeneic Tumor Models.

CD-1 Swiss mice and C57BL/6N mice were purchased from Charles River Laboratory. Animals were housed in a temperature-controlled room under a 12 hr light/12 hr dark cycle and fed normal chow diet. All animal studies were reviewed and approved by The Ohio State University Institutional Laboratory Animal Care and Use Committee (IACUC). Both male and female mice were used for experiments.

MC-38 murine colorectal cancer cell line MC-38 syngeneic subcutaneous murine colorectal cancer model was developed by injecting one million MC-38 cells in PBS onto the right flank of each C57 mouse subcutaneously. Hepa1-6 syngeneic subcutaneous murine liver cancer model was developed by injecting one million Hepa1-6 cells in FBS onto the right flank of each C57 mouse subcutaneously. Tumor sizes and mice weights were monitored daily using a digital caliper and analytical scale, respectively.

### In Vivo Antitumor Activity Evaluation of Cationic Nanoemulsion Incorporating TLR Agonists on Hepa1-6 Murine HCC Syngeneic Model.

The treatments were started when the average tumor sizes reached 80-100 mm³. Mice were randomized into 4 groups with 5 mice per group. The treatments, including normal saline, TLR9a CNE (CpG 2216), TLR3a CNE (poly(I:C)), and TLR7a NE at a dose of 50, 50, and 100pg per injection, were given twice a week with a total of 8 doses. Mice were sacrificed upon the tumor sizes reached the early removal criteria and tumors were collected for further analysis.

### In Vivo Vaccine Efficacy Evaluation and Tumor Challenge on J558 Murine Myeloid Syngeneic Model.

Wild type (WT) Balb/c mice (2 mice/control group and 3 mice/peptide-containing group) were treated with 100 μL of the peptide-included/control vaccines subcutaneously for 7 days. Mice were bled for Pl-CTL (cytotoxic T lymphocytes, CD8⁺ T lymphocytes) detection 7 days after immunization, and mice were challenged with J558 murine myeloma cells on the 10^{th} day. Mice were sacrificed 40 days after immunization and spleens and tumors examined for activated PI-CTL using flow cytometry.

### In Vivo Antitumor Activity Evaluation of Standard Lipid Nanoparticles Incorporating CpG Oligonucleotides and Anti-PDL1 LNA Gapmers on MC-38 Murine Colon Cancer Syngeneic Model.

The treatments were started when the average tumor sizes reached 80-100 mm³. Mice were randomized into 6 groups with 5 mice per group. The treatments, including normal saline control, anti-PDL1 2′-OMe ASO gapmer in PBS, doxorubicin in PBS solution, SD-101 in LNP, anti-PDL1 ASO in LNP, SD-101 in LNP and anti-PDL1 ASO in LNP combination, and anti-PDL1 ASO in LNP with doxorubicin combination, were given once every three days with a total of 5 doses. Mice were sacrificed upon the tumor sizes reached the early removal criteria and tumors were collected for further analysis.

In Vivo Antitumor Activity Evaluation of pH-Sensitive Nanoemulsion (PSNE) Lipid Nanoparticles on MC-38 Murine Colon Cancer Syngeneic Model.

The treatments were started when the average tumor sizes reached 80-100 mm³. Mice were randomized into 6 groups with 5 mice per group. The treatments, including normal saline control, SD-101 oligonucleotides, SD-101 in PSNE, anti-PDL1 LNA in PSNE, SD-101 and anti-PDL1 LNA in PSNE, and Resiquimod and SD-101 in PSNE, were given once every three days with a total of 5 doses. Mice were sacrificed 6 hours after the final dose and various organs as well as whole blood samples were collected for further analysis.

In Vivo Antitumor Activity Evaluation of Next Generation pH-Sensitive Nanoemulsion (PSNE-Chol) Lipid Nanoparticles on MC-38 Murine Colon Cancer Syngeneic Model.

The treatments were started when the average tumor sizes reached 80-100 mm³. Mice were randomized into 5 groups with 6 mice per group. The treatments, including normal saline control, SD-101 in PSNE-Chol, anti-PDL1 LNA in PSNE-Chol, SD-101 and anti-PDL1 LNA in PSNE-Chol, and the mixture of SD-101 in PSNE-Chol and anti-PDL1 LNA in PSNE-Chol, were given once every three days with a total of 5 doses. Mice were sacrificed upon the tumor sizes reached the early removal criteria and various organs as well as whole blood samples were collected for further analysis.

### Splenocyte and Tumor Tissue Messenger RNA Quantification by RT-qPCR.

Splenocyte messenger RNA for gene expression analysis was extracted from splenocyte suspensions using TRI reagent per manufacturer's protocol. cDNA was prepared using High-Capacity cDNA Reverse Transcription Kit (Invitrogen). Real-time PCR was conducted on a QuantStudio 7 Flex Real-Time PCR System. The relative amount of RNA level was calculated and compared according to the 2−ΔΔCt method.

### Splenocytes and Tumor Infiltrated Immune Cells Population Examination by Flow Cytometry.

Single cell splenocyte suspensions were harvested from spleens by the following procedures. Spleens were gently meshed through 70 um nylon meshes (Thermo Scientific) using a sterile 5-mL syringe plunge and washed with cold RPMI medium twice into six-well plates. Crude splenocyte suspensions were then centrifuged at 500×g for 5 minutes at 4° C. to obtain cell pellets. Cell pellets were resuspended in 1×RBC lysis buffer to lyse red blood cells for 5 minutes at RT and 10-fold volume of PBS was added to stop the reaction. After 500×g centrifugation for 5 minutes, the cell pellets were then resuspended in FACS staining buffer to form single cell splenocyte suspensions.

Antibodies were purchased from Biolegend (San Diego, CA), and surface cell staining was done per manufacturer's protocol. Intracellular staining was done by using Biolegend True-Nuclear™ Transcription Factor Buffer Set per manufacture protocol. The stained cells were analyzed on a BD LSRForetessa Flow Cytometer in the low Cytometry Shared Resource Core at The Ohio State University Comprehensive Cancer Center.

### Cytokine ELISA.

Serum samples were obtained from whole blood samples by incubating blood at RT for 30 minutes, followed by 2,000×g centrifugation for 20 minutes. Sera were collected and stored at −80° C. prior to cytokine analysis. Cytokine concentrations were measured by uncoated ELISA kits purchased from Invitrogen per manufacturer's protocols.

### Statistical Analysis.

Statistical analyses were performed with GraphPad PRISM software version 9 (GraphPad Software, San Diego, CA, USA). P-values 0.05 were considered significant.

### Results

### In Vivo Antitumor Activity Evaluation of Cationic Nanoemulsion Incorporating TLR Agonists on Hepa1-6 Murine HCC Syngeneic Model.

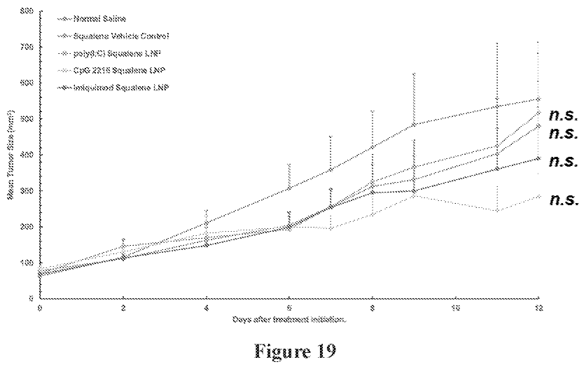

In the first preliminary study, several different toll-like receptor agonists were examined utilizing squalene-based nanoemulsions and intratumoral injections. Toll-like receptors are known to initiate immune responses as different receptors are responsible for sensing different kinds of PAMPs. Here, TLR3 agonist poly(I:C), TLR7 agonist imiquimod and TLR9 agonist CpG 2216 were selected and were encapsulated into the nanoemulsions either through hydrophobic interactions (imiquimod) or electrostatic interactions (poly(I:C) or CpG 2216). The drug nanoparticles were stable without forming aggregates while stored at 4° C. Due to the limited cavities in the tumor tissues, the injection volumes were limited to 50-100 μL per injection. The administrations were done by directly injecting the nanoemulsions into the tumor center cavities with careful monitoring. In FIG. 19, the tumor progression curves showed the apparent trends of the effect of TLR agonists in tumor growth suppression. Although the suppression of the tumor growth did not reach statistically significant, it was clear that CpG 2216, a TLR9 agonist and type A CpG oligodeoxynucleotide, worked the best among all in suppressing tumor growth. Surprisingly, poly(I:C), a TLR3 agonist shown to be able to trigger dendritic cell maturation and tumor regression, did not have an antitumor effect compared with the squalene vehicle control. The potency comparison of the TLR agonist squalene nanoemulsions was as follows: CpG 2216 (TLR9) being the more potent among all, then imiquimod (TLR7), and poly(I:C) (TLR3) being the least potent agent.

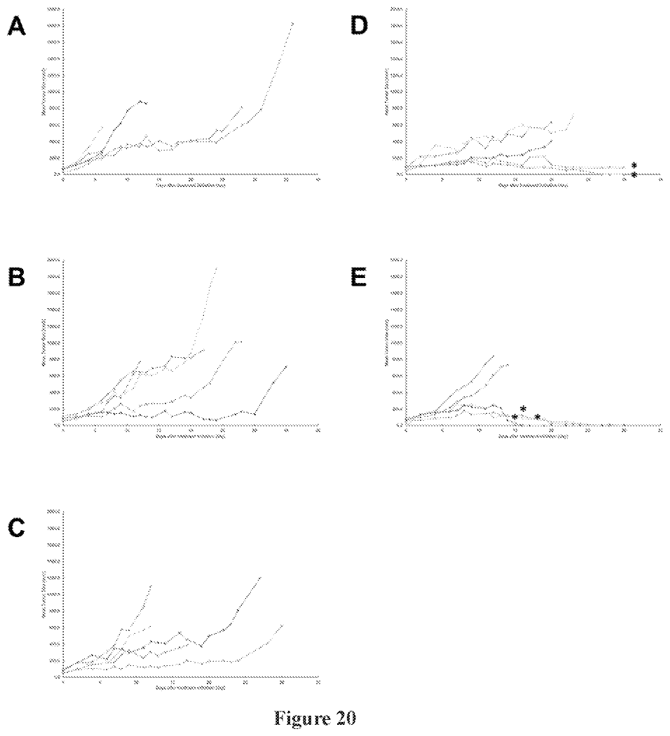

FIG. 19 was based on the treatment period before the early removal criteria. Since Hepa1-6 syngeneic tumor is an aggressive tumor during progression and may form bleeding ulcers at late stages, the mice had to be sacrificed early to meet the regulations. Therefore, if the survival of those well-conditioned mice were considered (FIG. 20), the results indicated that both CpG 2216 squalene nanoemulsions and imiquimod squalene nanoemulsion could lead to complete tumor regression on two or three mice, respectively (FIG. 20, Panel D and 20, Panel E). The antitumor outcome was promising, and additional tumor challenges were done on those complete regression mice. To perform tumor challenge on immunized mice, 1-2 million Hepa1-6 cells were injected subcutaneously at a similar site to the previous tumor, and the mice were monitored for additional two weeks for tumor uptake and progression. In naïve C57BL/6N mice, the cancer cells should be uptaken and progress rapidly after a week after inoculation. During the tumor challenge period, the monitoring time was extended to two weeks to ensure the total cleanout of the inoculated cancer cells. The results turned out that those five complete-tumor-regression mice were all free from tumor uptake after tumor challenge, suggesting that the mice developed anticancer immunity specifically against Hepa1-6 HCC.

### In Vivo Cancer Vaccine Efficacy Evaluation and Tumor Challenge on J558 Murine Myeloid Syngeneic Model.

In the first preliminary study, only TLR agonists were supplied utilizing nanoparticle carriers. The tumor-associated antigens were supplied endogenously within the tumor microenvironment. During the intratumoral injection, dendritic cells and other phagocytes could uptake both TLR agonist nanoparticles and/or tumor-associated antigens to initiate inflammatory responses and dendritic cell activation or maturation. Antigen presentation in dendritic cells is a highly specified and regulated process that requires degraded pathogen proteins (antigens) and activation signals (TLR agonists). Plasmacytoid dendritic cells (pDC) activated by CpG TLR9 agonists are professional antigen-presenting cells (APC) to CD8⁺ T lymphocytes through cross antigen presentation. However, a successful antigen presentation procedure requires the simultaneous occurrence of PAMP (here, TLR agonists) and antigen proteins in APCs. Therefore, to develop a well-defined cancer vaccine against a specific cancer subtype, a specific, well-defined antigen peptide was added to the squalene nanoemulsion, along with the combinations of TLR agonists in the second preliminary study.

Since the single TLR activation did not give substantial responses, dual or triple TLR agonist combinations were considered. Poly(I:C) was abandoned due to its low potency. MPLA, which is a TLR4 agonist working similarly to lipopolysaccharide (LPS) by mimicking bacterial infections, was selected as one of the components. Besides, imiquimod was substituted by the modified version, Resiquimod, which is a TLR7/8 agonist, to enhance the drug solubility and improve the process manipulation. Furthermore, SD-101 CpG oligonucleotide, a type-C CpG oligonucleotide, was added to the portfolio along with CpG 2216, a type-A CpG oligonucleotide. The evaluation of the potency of each combination (Table 2) was done by immunizing the mice with peptide-incorporated TLR agonist nanoemulsions subcutaneously, followed by tumor challenge.

TABLE 2
TLR agonist composition of cationic nanoemulsions incorporating
tumor antigen peptides and TLR agonists.

| | Positive | | | | | |
|---|---|---|---|---|---|---|
| μg/injection | Control | 1 | 2 | 3 | 4 | 5 |
| TLR4a, MPLA | 20 | 0 | 30 | 20 | 0 | 0 |
| TLR7/8a, Resiquimod | 20 | 30 | 0 | 20 | 30 | 0 |
| TLR9a, CpG-2216 | 20 | 30 | 30 | 20 w/.SD-101 | 30 w/.SD-101 | 50 w/.SD-101 |

P1A is a well-defined antigen peptide for murine myeloma cells J558. The peptide can be synthesized by solid-state peptide synthesis with high purity and high precision. The sequence of the P1A peptide is LPYLGWLVF (SEQ ID:3), with over half of the residues being hydrophobic or aromatic amino acids (highlighted bold). The high lipophilicity of P1A antigen peptide was be dissolved in the squalene core and formed cationic nanoemulsions by adding permanently charged DOTAP to the formulations. DOTAP was used as the electrostatic charge source to pair with oligonucleotides to form stable cationic nanoparticles or nano-lipoplex.

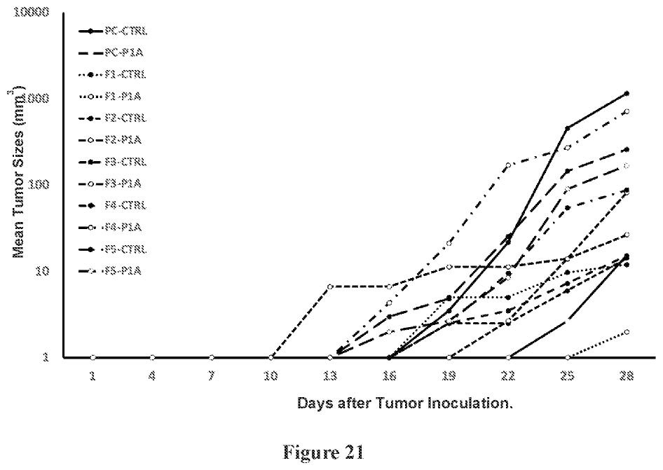

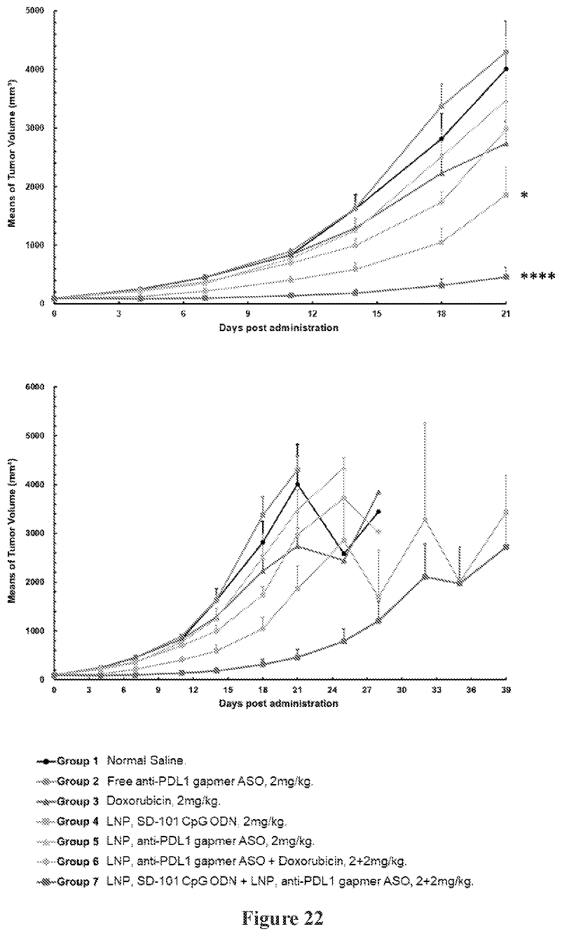

Worked as the cancer vaccine candidate, the mice were immunized once to illustrate the immune system stimulation potency, and a tumor challenge was done to demonstrate the protective ability after immunization. In FIG. 21, the result clearly showed that the PC, which was the combination of CpG 2216, MPLA, and Resiquimod at a weight ratio of 1:1:1, could generate J558-specific anti-peptide cytotoxic T lymphocytes, and that led to the delayed growth of J558 tumor during the tumor challenge process. Other combinations, such as F1 to F3 with dual TLR agonists at single lipid nanoparticles along with the PIA antigen peptides, showed substantial activation of J558-specific immunity and could delay, or eliminate, the onset of the J558 challenge. The preliminary study using TLR agonist combinations, and the addition of tumor-specific antigen peptides indicated that the enhancement of cancer vaccines relied on the co-delivery of tumor-associated antigens and TLR agonists as the crucial stimulating factors.

### In Vivo Antitumor Activity Evaluation of Standard Lipid Nanoparticles Incorporating CpG Oligonucleotides and 2′-OMe Anti-PDL1 Gapmers on MC-38 Murine Colon Cancer Syngeneic Model.

In a third study, SD-101 was combined with anti-PDL1 antisense oligonucleotides (anti-PDL1 ASO) to examine the antitumor efficacy by not only stimulating immune responses but also downregulating the checkpoint molecule PD-L1, or programmed cell death-ligand 1. PD-L1, also known as cluster of differentiation 274 (CD274), is a protein that acts as a brake to stop the immune responses by binding to its receptor PD-1 and inducing inhibitory signals. By downregulating the PD-L1 expression in either immune cells or cancer cells, T lymphocyte responses would not be diminished by the signaling pathways triggered by PD-1/PD-L1 interactions. To demonstrate the activity of both SD-101 CpG ODN and anti-PDL1 ASO, both oligonucleotides were fully encapsulated in standard lipid nanoparticles with the golden standard ionizable lipid DLin-MC3-DMA at a golden composition of DSPC/cholesterol/DLin-MC3-DMA/DMG-mPEG₂₀₀₀=10/38.5/50/1.5 (m/m). Mice bearing with MC-38 tumors were treated once every three days for five doses. The results (FIG. 22) demonstrating that the mice treated with SD-101 LNP or the combination of SD-101 LNP and anti-PDL1 ASO LNP had significant tumor regression 21 days after treatment initiation over saline controls suggesting that the overall immunosuppressive environment in tumor-bearing animals could be reversed by applying TLR agonists systemically. On the other hand, anti-PDL1 ASO only or anti-PDL1 ASO LNP only group did not show significant tumor growth suppression over saline controls. Moreover, while comparing the tumor burden of the mice treated with doxorubicin with those treated with the combination of doxorubicin and anti-PDL1 ASO LNP, the outcome indicated that anti-PDL1 ASO LNP had no therapeutic activities over doxorubicin chemotherapeutics. When the monitoring period was extended three weeks after the final doses (FIG. 22), the results showed that the mice treated with the LNP combinations had a slower tumor growth rate, but the mice would eventually be suffered from the large tumor burden, and no preventive or curative effect was developed by the combination.

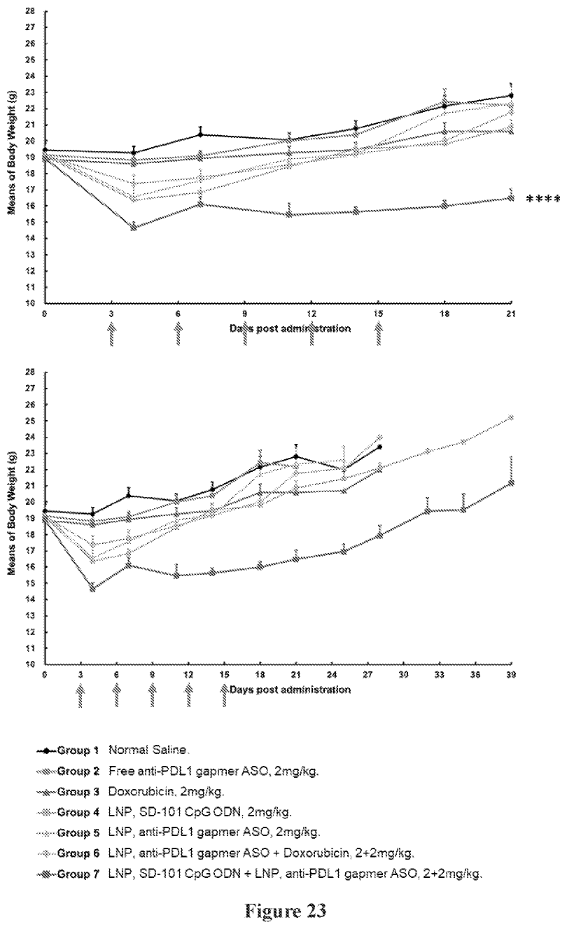

Besides the tumor burden analysis, the weight analysis during the treatment period was an important parameter to evaluate the overall systemic toxicity caused by the therapeutic agents. In FIG. 23, the result clearly showed that mice of the LNP combination treatment group had suffered from significant weight loss during the treatment period (the initial 15 days), and the treatment had to be paused due to the significant toxicity. Moreover, the LNP single treatment groups both experienced at least 10% weight loss during the first dose, and that suggested the LNP platform had significant intrinsic toxicity, which was reported before that the LNPs were known to trigger cytokine release syndrome that might deteriorate the mice health conditions. However, those mice treated with LNP single agents were adopted to the LNP carrier as the treatment continued and had a similar weight gain profile as the saline controls. The overall results suggested that SD-101 LNP treatment was an effective treatment option against cancer, while the 2′-OMe anti-PDL1 gapmer ASO had a minor effect on tumor regression.

### In Vitro Gene Regulation Activity Evaluation of Anti-PDL1 Gapmer ASO by RT-qPCR.

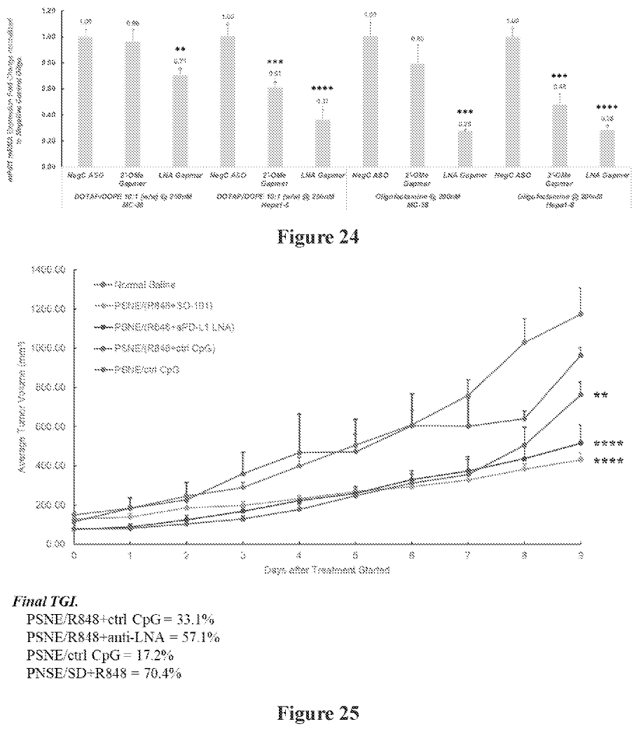

The anti-PDL1 gapmer ASO was originally designed by Roche in locked nucleic acid (LNA) modifications at two ends and tagged with a GalNac moiety for liver targeting. In our study, the GalNac moiety was removed to obtain a single-stranded, 16-mer anti-murine PD-L1 gapmer antisense oligonucleotide. In the initial testing, 2′-OMe modified nucleotides were used at two ends to protect the antisense oligonucleotides from nuclease degradation. However, studies have shown that 2′-OMe modification had minor improvement over the original deoxynucleotides, with minor enhancement in messenger RNA binding. The newer version of nucleotide modification is the locked nucleic acid (LNA), in which the 2′-O on the ribose is linked with the 4′-C through the methylene bridge. The binding affinity of the LNA modified ASO is significantly stronger than 2′-OMe modified ASO, and the former can increase RNase H binding to the complementary sequences after binding with target messenger RNA for mRNA degradation. Therefore, the target gene downregulation of the original design of the LNA-modified anti-murine PD-L1 gapmer ASO was examined against the 2′-OMe modified ASO through different transfection methods on different murine cell lines in vitro. In FIG. 24, the evaluation was done using either DOTAP/DOPE as the transfection agent in serum-free media or oligofectamine in complete media on Hepa1-6 murine HCC cell line and MC-38 murine colon cancer cell line. The results indicated that LNA-modified ASO had higher gene downregulation activity over the 2′-OMe version, and the downregulation efficiency was a cell line-dependent process. It was clearly shown that the Pdl1 gene in MC-38 could not be downregulated easily as that in Hepa1-6, and the trend was shown in both DOTAP/DOPE and oligofectamine-treated cells. In short, to get a better therapeutic response by downregulating PD-L 1 in murine tissues, LNA-modified ASO had to be introduced to the LNP construct to ensure the substantial downregulation of the target gene.

In Vivo Antitumor Activity Evaluation of pH-Sensitive Nanoemulsion (PSNE) Lipid Nanoparticles on MC-38 Murine Colon Cancer Syngeneic Model.

To evaluate the anticancer activity of the PSNE lipid nanoparticles encapsulating SD-101 CpG ODN, anti-PDL1 LNA ASO, and Resiquimod in vivo, mice were treated with the single agents or combinations at doses of 2.0 mg/kg each once every three days for five doses. In FIG. 25, the tumor growth curves showed that the PSNE encapsulated Resiquimod had minor effects on tumor growth suppression, while the combinations, including Resiquimod with anti-PDL1 LNA ASO and Resiquimod with SD-101 CpG oligonucleotides, had significant effects in tumor growth inhibition, with TGIs around 57.1% and 70.4%, respectively.

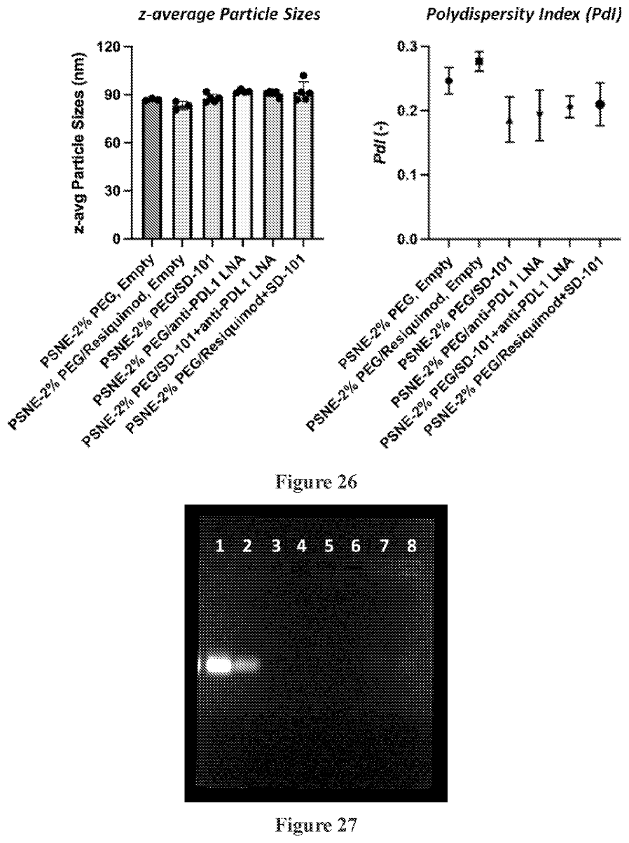

In the second trial, different parameters and characterizations of PSNE lipid nanoparticles were examined and evaluated. PSNE lipid nanoparticles encapsulating oligonucleotides and small molecules were successfully formulated with high colloidal stability. Small particle sizes ˜80-100 nm were achieved for both empty PSNE LNP and encapsulated LNP with either SD-101 CpG ODNs or anti-PDL1 LNA ASOs. The polydispersity indexes (Pd1) were ˜0.15-0.25, indicating uniformly distributed particle sizes were obtained in all samples (FIG. 26). Empty PSNE lipid nanoparticles had a mean zeta potential at +8.06 mV in 20 mM PB at pH 4 and encapsulated PSNE lipid nanoparticles had mean zeta potentials around neutral in 10 mM PB at pH 7.

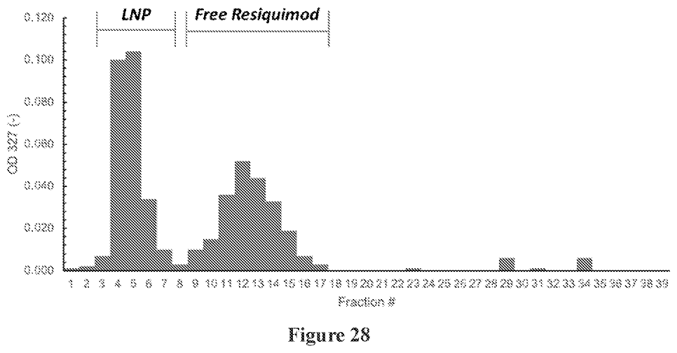

Next, the encapsulations of ODN and small hydrophobic molecule cargos in PSNE LNP were analyzed by gel retardation assay (FIG. 27) and size exclusion chromatography (FIG. 28), respectively. The gel image showed that ODNs were highly encapsulated in PSNE LNP, with a minor amount of SD-101 adsorbed onto the PSNE surface while Resiquimod was added to the formulation. The encapsulation percentage of Resiquimod in PSNE lipid nanoparticles was ˜53.8% by calculating the ratios of two peaks on the chromatogram obtained by OD327 using a NanoDrop spectrophotometer (FIG. 28).

TABLE 3
Lane description of the samples loaded on
agarose gel retardation assay (FIG. 27).

| Lane | |
|---|---|
| 1 | SD-101 |
| 2 | anti-PDL1 LNA |
| 3 | PSNE-2% PEG |
| 4 | PSNE-2%/R848 |
| 5 | PSNE/SD-101 |
| 6 | PSNE/LNA |
| 7 | PSNE/SD + LNA |
| 8 | PSNE/R848 + SD |

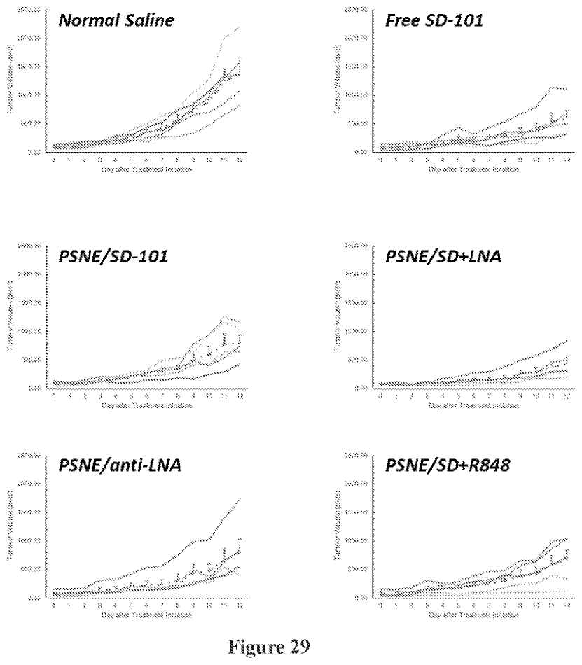

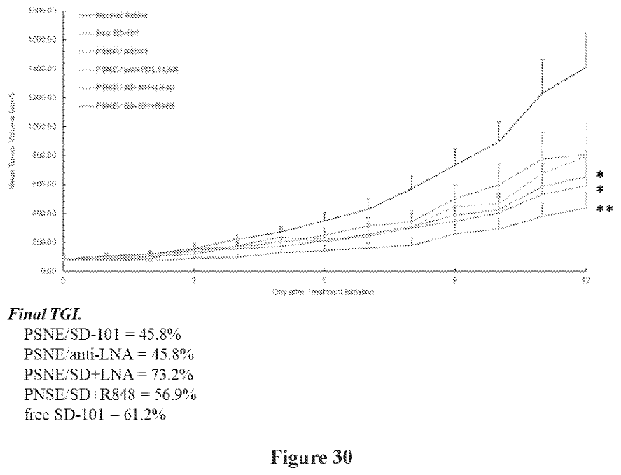

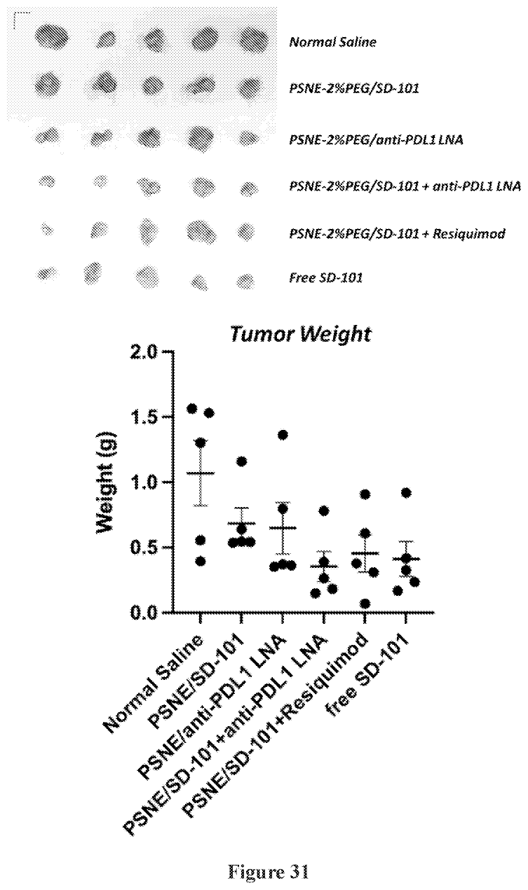

The combination of SD-101 CpG ODNs and anti-PDL1 LNA ASOs in PSNE lipid nanoparticles showed a good tumor growth inhibition outcome on the MC-38 murine syngeneic colon cancer model (FIG. 29, 30) with a mean tumor growth inhibition (TGI) ˜73%. Two groups of mice treated with single-agent in PSNE (either SD-101 ODNs or anti-PDL1 LNA ASOs) both showed TGI ˜46%, while mice treated with PSNE encapsulating SD-101 and Resiquimod had a mean TGI of 57% (FIG. 30). The tumor weights were recorded and represented the actual tumor sizes after the treatments. The results showed a similar trend with the tumor volumes, as the PSNE combination group showed significant tumor growth inhibition compared to normal saline control using one-way ANOVA. (FIG. 31)

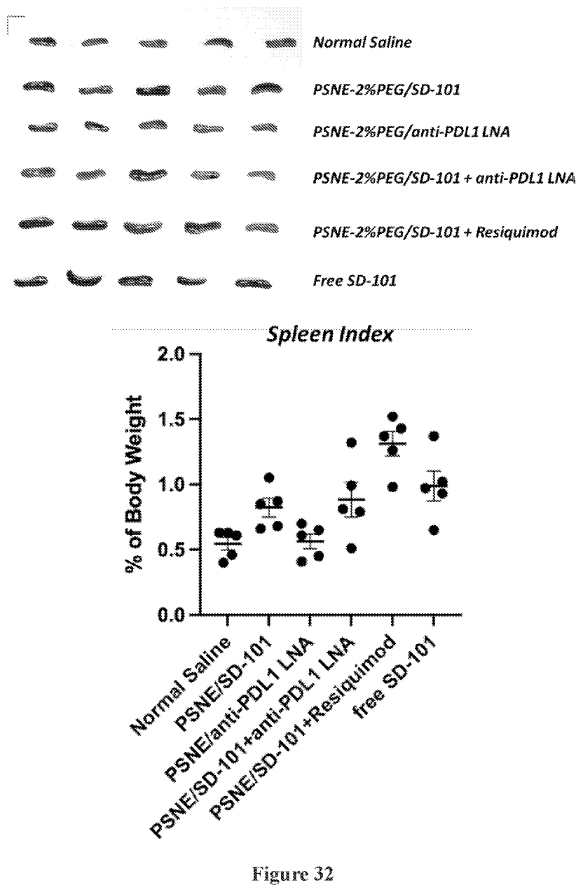

The spleen index (spleen weight presented in the percentage of body weight, FIG. 32) result indicated that PSNE has a similar immune system activation ability as SD-101 oligonucleotides. Also, treating dual TLR agonists in combinations did further activate the immune system as well (by expanding immune cell populations). Besides, the results also indicated that PSNE lipid nanoparticles encapsulating anti-PDL1 LNA ASOs did not have immune system activation ability. The tumor growth inhibition of PSNE/anti-PDL1 LNA ASOs could be led by the PD-L 1 downregulations in tumor sites, either LNA delivery to cancer cells or TME immune cells.

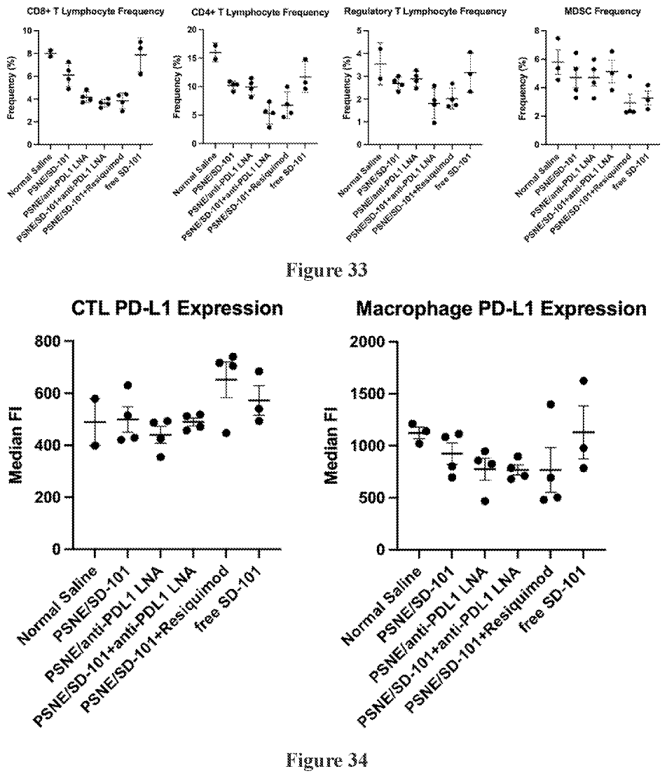

In flow cytometry analysis (FIG. 33), all PNSE-based treatment groups had significant decreases in T lymphocyte population frequencies, either CD4⁺ T helper cells, CD8⁺ cytotoxic T cells, or regulatory T cells, compared to the normal saline control group. The results could be described as T cell activation, therefore, infiltrating to TME for immune reactions. IHC staining T cell populations can be done to verify the hypothesis. MDSC frequencies had no significant downregulations after all treatments in the MC-38 tumor model.

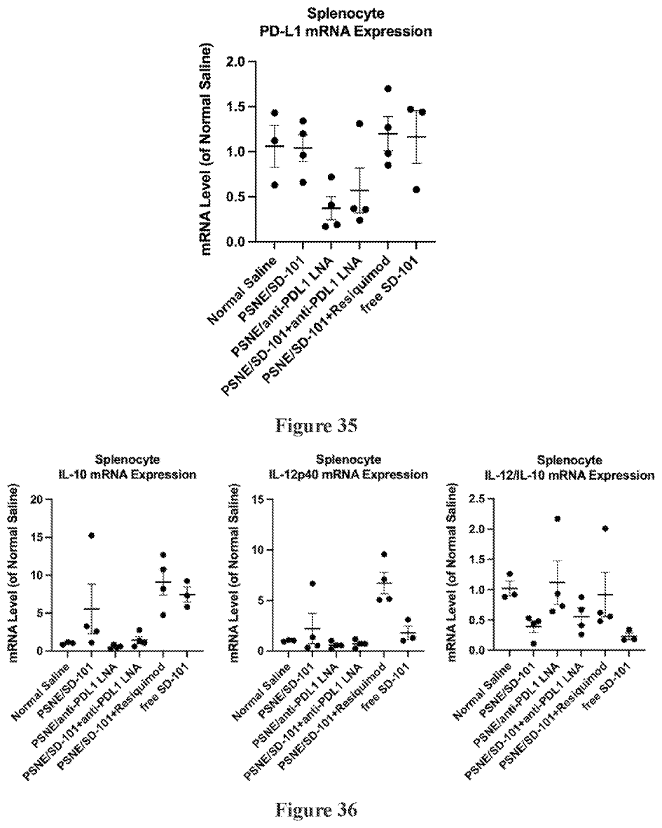

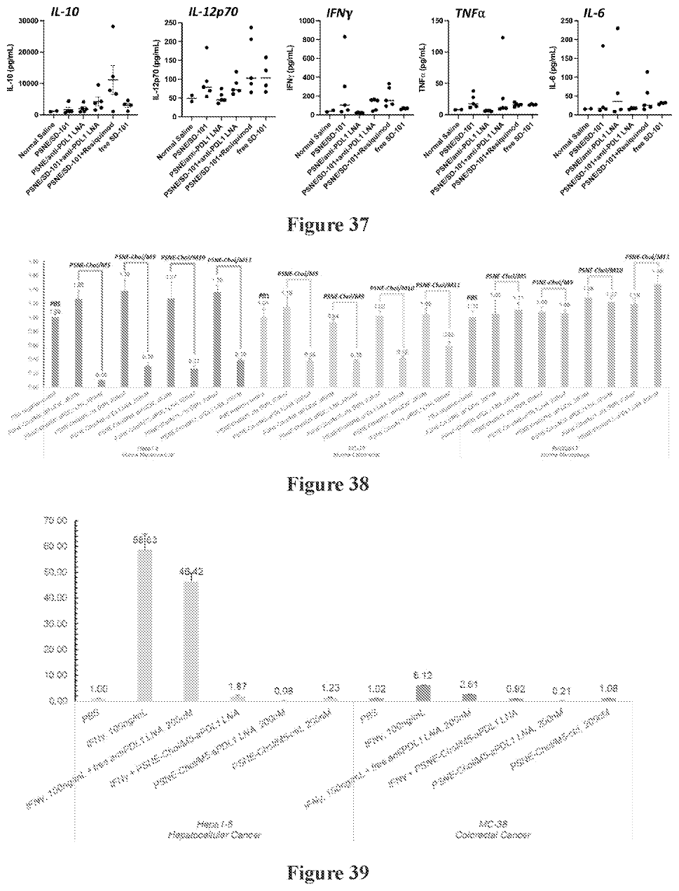

Surface PD-L1 markers on both lymphocytes and myeloid cells were examined by flow cytometry to investigate the PD-L 1 protein expressions on immune cells after treatments (FIG. 34). The results showed that surface PD-L1 protein levels on macrophages were downregulated in groups treated with anti-PDL1 LNA ASOs, given that macrophages had the ability to phagocytose LNPs and uptake LNA for mRNA downregulation. Nonetheless, PD-Li protein expressions on cytotoxic T lymphocytes did not have significant downregulations after being treated with anti-PDL1 LNAs, indicating the difficulties of transfecting T cells. As we expected, PD-L1 protein expression levels in TLR (SD-101 ODNs and Resiquimod) treatment groups were upregulated due to the NFκB-MyD88 activation pathway. This also indicated that anti-PDL1 LNA is able to overcome the PD-L1 upregulation by TLR agonists. By doing splenocyte mRNA analysis, we could get ideas of the cytokine secretion profile as well as overall PD-L1 levels after treating with TLR agonists and/or anti-PDL1 LNA. The results demonstrated the excellent activity of LNA in downregulating PD-L1 mRNA among splenocytes (FIG. 35). Based on the cytokine ELISA results (FIG. 36, 37), we could easily notice that mice treated with PSNE/anti-PDL1 LNA were not in an inflammatory state (basal levels of pro-inflammatory cytokines TNF-α and IFN-γ); therefore, the tumor growth inhibition was related to the anti-PDL1 LNA ASO delivery to the tumors. Also, the results also showed that TLR agonists were able to prime the immune system toward pro-inflammatory stage by elevating IL-12 levels.

### In Vitro Gene Regulation Evaluation by RT-qPCR.

The PSNE was further developed by applying the knowledge from the COVID-19 vaccines in lipid nanoparticle delivery. The next-generation PSNE was composited of DOPE, squalene, DLin-MC3-DMA, cholesterol, and DMG-mPEG₂₀₀₀. The evaluation of the gene delivery (antisense oligonucleotides, siRNAs, mRNAs) was done by RT-qPCR in vitro and Luciferase bioluminescence in vivo (data not shown). The compositions were fine-tuned, and 11 different formulations were examined and presented as a series of next-generation PSNE for different applications. Based on the evaluation results (data not shown), there were several candidates that had high in vivo expression administered systemically, such as PSNE-Chol/M5. Additionally, PSNE-Chol/M9-M11 were tested in vitro along with M5 as M9, M10, and MI I showed high mRNA delivery efficiency in vitro on various cell lines (HEK293T, A549, Hepa1-6, and HepG2).

In the gene delivery evaluation study, three different signature murine cell lines were used, including Hepa1-6 HCC cell line, MC-38 colon cancer cell line, and RAW264.7 macrophage cell line. The former two represented the tumor burden while the latter represented the immune cell populations. All the gene expression levels were normalized to the PBS control group, giving us a clear comparison among each treatment group. As shown in FIG. 38, the result again indicated that Hepa1-6 was more sensitive to anti-PDL1 LNA ASO treatments using various formats. Surprisingly, all next-gen PSNE platforms would induce murine PD-L 1 expression in Hepa1-6, but the upregulations were significantly inhibited by the anti-PDL1 LNA ASO. That is, the high gene regulation potency of murine PD-L 1 was shown by using next-gen PSNE as the delivery platform. Compared with Hepa1-6, MC-38 was not highly sensitive to the anti-PDL1 LNA ASO treatment, as shown in the previous section. However, in both cell lines, it was obvious that PSNE-Chol/M5 had the best overall murine PD-L1 downregulation efficiency among all next-gen PSNE tested.

On the other hand, the result on RAW cells suggested that immune cells were not susceptible to anti-PDL1 LNA ASO gene regulation using next-gen PSNEs. The expression level of the murine PD-L1 had no significant changes while treated with control oligonucleotides or anti-PDL1 LNA ASO. The murine PD-L1 expressions were slightly upregulated in each treatment groups indicating that the next gen PSNE platforms might have the ability to activate RAW cells, such as cytokine release, which was reported for most lipid nanoparticle formulations.

Next, the next-gen PSNE, specifically PSNE-Chol/M5, was tested in both Hepa1-6 and MC-38 in vitro to evaluate the murine PD-L1 downregulation efficiency under the stimulation of cytokines. Cancer cells are highly sensitive to the surrounding cytokines and chemokines and will respond to the environmental stimulations accordingly. It is well-known that pro-inflammatory cytokine interferon gamma (IFN-γ) would induce surface PD-L1 expression on cancer cells in response to the pro-inflammation status and activation of adaptive immune systems. In FIG. 39, the RT-qPCR result confirmed that both Hepa1-6 and MC-38 were responsive to IFN-γ induction in PD-L1 expression, and Hepa1-6 was more predominant in the upregulation. Next, the PSNE-Chol/M5 LNP encapsulated with anti-PDL1 LNA ASO was treated along with IFN-γ to validate the downregulation efficacy of the LNPs. The outcome indicated that PSNE-Chol/M5 could successfully deliver anti-PDL1 LNA ASO to the cells and downregulated PD-L 1 mRNA expression back to basal level after the induction of IFN-γ. In addition, the RT-qPCR results suggested that even free anti-PDL1 LNA antisense oligonucleotides could be uptaken by cells at a certain level and resulted in a significant downregulation in PD-L1 mRNA, but not as efficient as the PSNE-Chol/M5 delivery platform. Overall, the RT-qPCR demonstrated the solid efficacy of gene-level regulation of PSNE-Chol/M5 LNP with anti-PDL1 LNA ASO, even on the cells with PD-L1 induction by pro-inflammatory cytokines

### In Vitro Surface Protein Expression Evaluation by Flow Cytometry.

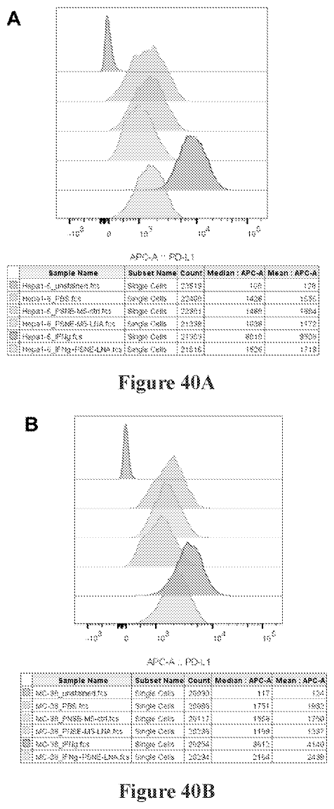

In the previous section, the gene regulation of murine PD-Li mRNA by IFN-γ and/or PSNE-Chol/M5-anti-PDL1 LNA ASO was confirmed by RT-qPCR, and the result demonstrated the promising outcome of using PSNE-Chol/M5-anti-PDL1 LNA ASO as an agent to decrease the expression level of mRNA at mRNA stage. However, the protein expression level of the surface PD-L1 must be confirmed to further demonstrate the correlation of using PSNE-Chol/M5-anti-PDL1 LNA ASO as an anticancer agent in regulating immune checkpoint expression. To investigate the surface PD-L1 expression on cancer cells and immune cells, Hepa1-6 and MC-38 cells treated with IFN-7 and PSNE-Chol/M5-anti-PDL1 LNA ASO were scanned using flow cytometry to quantify the surface immune checkpoint expression. Moreover, RAW264.7 cells treated with PSNE-Chol/M5-SD-101 were examined for the surface activation marker CD86 as well as surface PD-L1 expression. In FIGS. 40A-40B, the results illustrated that the surface expression of PD-L1 was highly correlated with the gene expression results obtained from RT-qPCR. There was a clear upshift of protein expression on Hepa1-6 while incubated with IFN-γ and the protein expression was suppressed by PSNE-Chol/M5-anti-PDL1 LNA ASO. The cells treated with IFN-γ and free anti-PDL1 LNA ASO had no significant changes in surface marker expression compared with the IFN-γ-treated positive control group, which indicated that free antisense oligonucleotides had minor effect in downregulating target gene without any delivery or targeting ligands or platforms. On the other hand, the increase of surface PD-L1 expression was less significant in MC-38 cells compared with Hepa1-6 cells, which correlated with the RT-qPCR data as well. However, the protein expression in MC-38 after IFN-γ induction could not be fully reversed by PSNE-Chol/M5-anti-PDL1 LNA ASO, even at a higher treatment level of 200 nM. The overall data suggested that MC-38 might not be a good model for PSNE-Chol/M5-anti-PDL1 LNA ASO with elevated expression of PD-L1 expression.

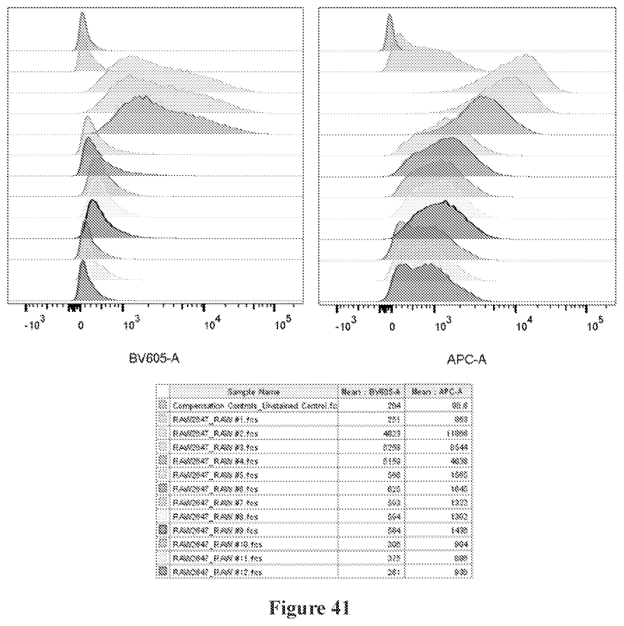

RAW264.7 cell surface markers were examined after the treatments to investigate the immune activation effect of each agent and combination. As a positive control of macrophage activation, LPS, a TLR4 agonist, showed a significant increase in CD86 expression as well as PD-L1 expression after the overnight treatment. However, the cells treated with free SD-101 oligonucleotides, a TLR9 agonist, did not exhibit strong activation (increase in surface CD86 expression) as LPS at the 5pg/mL level. On the other hand, RAW cells treated with LPS and PSNE-Chol/M5-anti-PDL1 LNA ASO showed a decrease in PD-L1 expression compared with LPS only group but were still significantly induced in PD-L1 expression by LPS (FIG. 41).

### Cytotoxicity Under Macrophage Condition Media

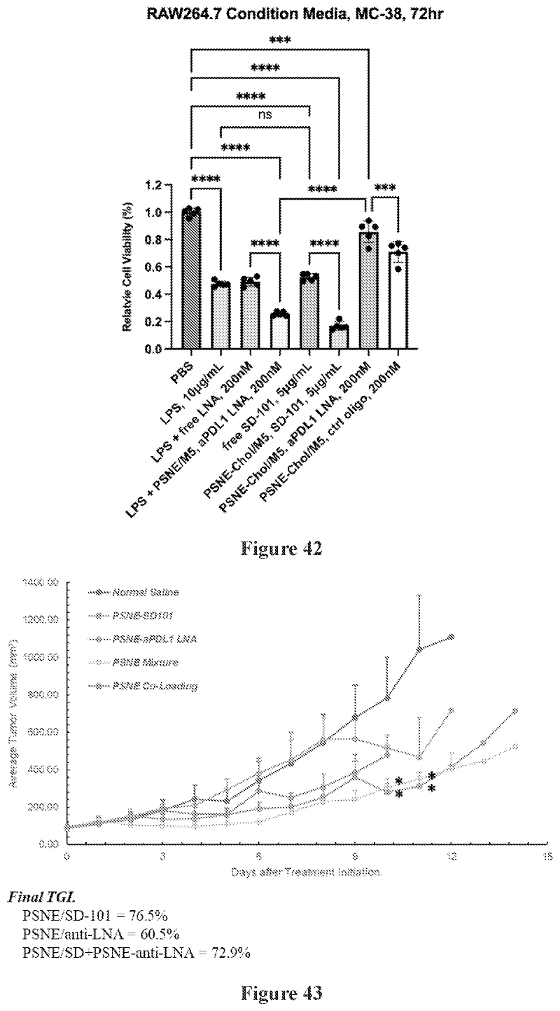

After verifying the direct effect of PSNE-Chol/M5-SD-101 LNP and PSNE-Chol/M5-anti-PDL1 LNA ASO LNP formulations on cancer cells and macrophages through RT-qPCR and flow cytometry, the indirect effects of PSNE-Chol/M5-SD-101 and PSNE-Chol/M5-anti-PDL1 LNA ASO LNP formulations on cancer cells were determined by examining the cancer cell viability after incubated with macrophage condition media. Condition media were prepared by harvesting the cytokine-containing media from macrophages treated with next-gen PSNE formulations overnight. The short-lived cytokines secreted by macrophages were preserved and directly interacted with cancer cells. Cancer cells, both Hepa1-6 and MC-38, were treated with RAW264.7 macrophage cytokine-containing condition media, and the viability was taken after 72 hours. Interestingly, Hepa1-6 (data not shown) has no significant cytotoxic effect on drug-treated condition media compared with normal condition media (PBS control). The morphology of Hepa1-6 cells was found to no change, or unaffected, under a bright-view microscope. On the other hand, MC-38 cells (FIG. 42) were more sensitive to the condition media treatments, and cells undergo apoptosis or necrosis in morphology, examined under a bright-view microscope. The positive control group, which was the cells treated with LPS condition media that was known to contain a substantial amount of cytotoxic cytokine TNFα, could induce significant cancer cell death after 72-hour incubation. The addition of free anti-PDL1 LNA ASO to the macrophage treatment made no changes to the cytotoxic effect on MC-38, but the addition of PSNE-Chol/M5-anti-PDL1 LNA ASO LNP along with LPS could further secret more cytotoxic cytokines, might or might not be limited to TNF-α, and cause higher cell death rate. Next, free SD-101 CpG oligonucleotide treatment was compared with LPS, and the result suggested that 5pg/mL of SD-101 CpG oligonucleotides had a similar effect as 10 μg/mL of LPS in the condition media treatment. Furthermore, while encapsulating SD-101 oligonucleotides into next-gen PSNE (PSNE-Chol/M5), the nanoparticle carrier effect further added to the responses and further enhanced the cell-killing effect of the condition media, and the carrier effect was also shown on the cells treated with the condition media from the macrophages treated with either PSNE-Chol/M5-anti-PDL1 LNA ASO or PSNE-Chol/M5-control ODN, which has significant cytotoxic effects on MC-38 compared with the PBS condition media or fresh complete media.

In Vivo Antitumor Activity Evaluation of Next Generation pH-Sensitive Nanoemulsion (PSNE-Chol) Lipid Nanoparticles on MC-38 Murine Colon Cancer Syngeneic Model.

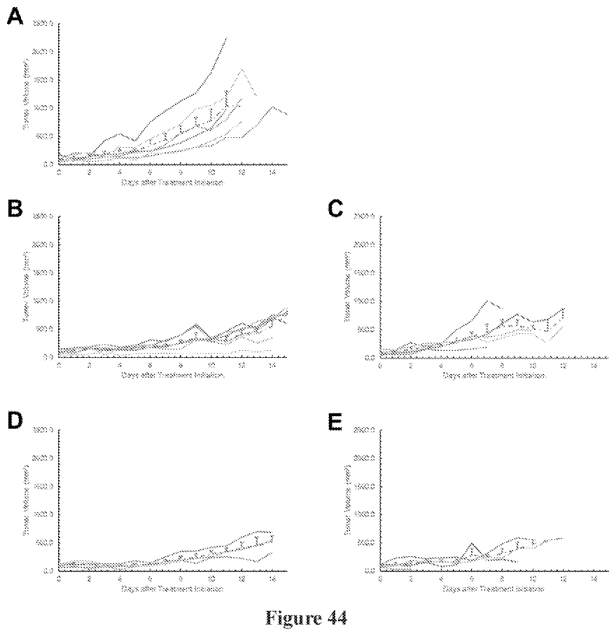

The in vitro validation of next-gen PSNE LNPs in cancer cell lines and macrophage cell lines gave us some insight into the therapeutic effects in vivo. Therefore, next-gen PSNE LNPs incorporating SD-101 and anti-PDL1 LNA ASO were tested on the MC-38 syngeneic model as single agents or as combinations. Based on the hypothesis, the anticancer efficacy of the next-gen PSNE should be higher than the previous generation PSNE without cholesterol. The delivery efficiency of anti-PDL1 LNA ASO to cancer cells and splenocytes should be higher than that using previous generation PSNE as well. The mice were treated with either saline control, PSNE-Chol/M5-SD-101 LNPs, PSNE-Chol/M5-anti-PDL1 LNA ASO LNPs, the 1:1 mixture of both LNPs, or the co-loading LNPs with the oligonucleotides at a weight ratio of 1:1. In FIGS. 43 and 44, the tumor growth curve showed that, again, the PSNE-Chol/M5-anti-PDL1 LNA ASO LNP single-agent treatment group did not work as efficiently as PSNE-Chol/M5-SD-101 LNP single-agent treatment group in suppressing MC-38 tumor growth. Also, none of the combination groups worked better than the PSNE-Chol/M5-SD-101 LNP single agent. The tumor growth inhibition (TGI) rates of the PSNE-Chol/M5-SD-101 LNP, PSNE-Chol/M5-anti-PDL1 LNA ASO LNP, and the mixture combination were 76.5%, 60.5%, and 72.9%, respectively. Both PSNE-Chol/M5-SD-101 LNP and the mixture combination group showed significant tumor growth inhibition in vivo compared with the saline control group. Moreover, from FIG. 43, the tumor growth curves suggested that these two groups could suppress the MC-38 tumor growth during the treatment period, but the tumors would start progressing after the treatment stopped.

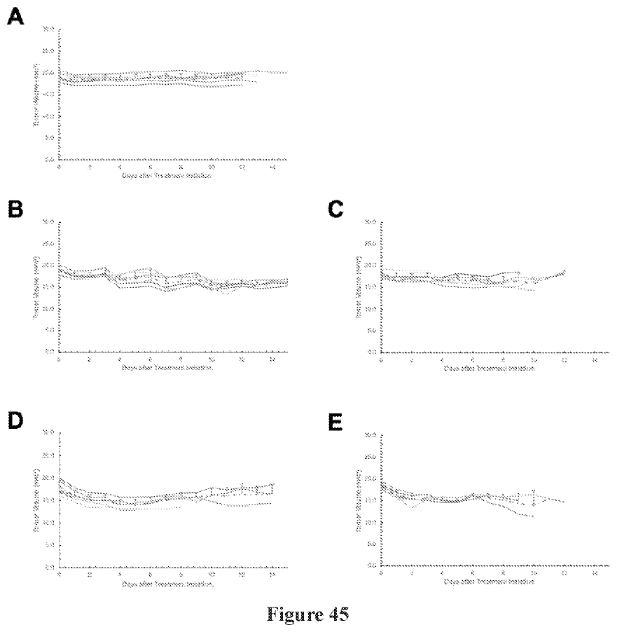

However, the toxicity of the LNP agents was monitored by the body weight loss and the early-stage viability during the treatment period. The body weight curves (FIG. 45) showed that the original combination groups (including mixture combination and co-loading combination) had significant weight loss of around 20% after the first dose and led to several early-stage death on the third day of the treatments, indicating the severe toxicity resulted from the LNP delivery platform, especially DLin-MC3-DMA ionizable lipid, which was shown in the previous section that the high-dose of DLin-MC3-DMA containing LNPs could lead to severe toxicity. The toxicity might come from the cytokine release syndrome or off-targeting effects on vital tissues. The combination groups were treated with reduced (half) doses to manage the delivery platform toxicity, which had the same lipid dose as the single-agent group, starting from the second dose, and all the mice that survived could tolerate the drug effect until treatment termination.

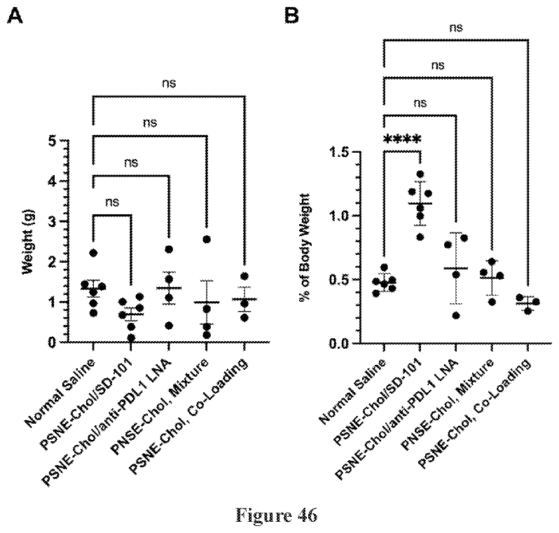

After the treatments were finished and the mice were sacrificed, several important parameters were measured and examined (FIG. 46) to investigate the possible mechanisms for tumor growth inhibition. Since the mice were sacrificed for survival at the point meeting the removal criteria, i.e., 1.6 cm in length, the average tumor sizes did not have a significant difference upon post-sacrifice measurement, still, PSNE-Chol/M5-SD-101 LNP single-agent group showed lower tumor burden in average. Nonetheless, while examining for splenomegaly, the result was clear that TLR agonist treatments could induce immune cell proliferation, leading to spleen enlargement. As illustrated in FIG. 46, Panel B, PSNE-Chol/M5-SD-101 LNP single-agent group exhibited a significant increase in spleen sizes after five doses of treatment, which matched the previous trend but worked even better than the dual TLR agonist group using first-gen PSNE LNPs. The finding indicated that next-gen PSNE could induce immune system activation at a greater level.

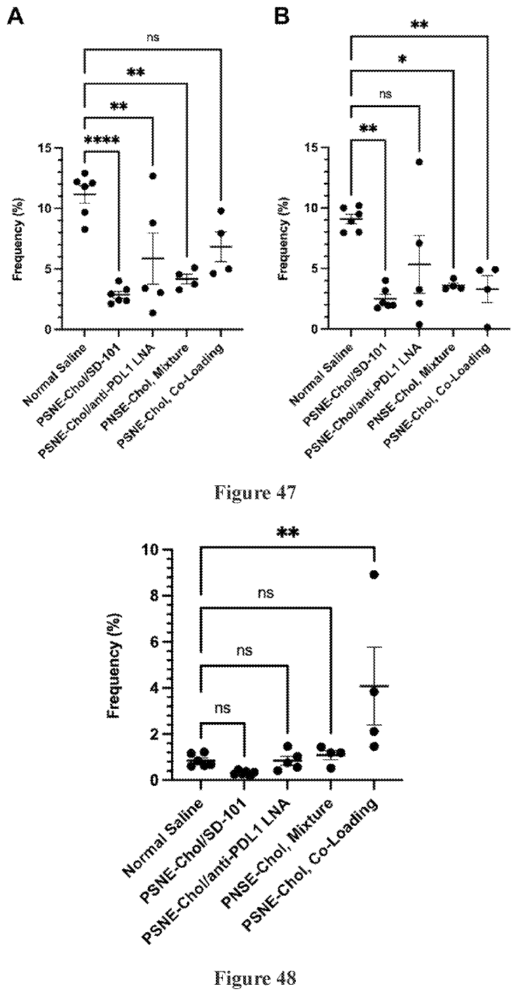

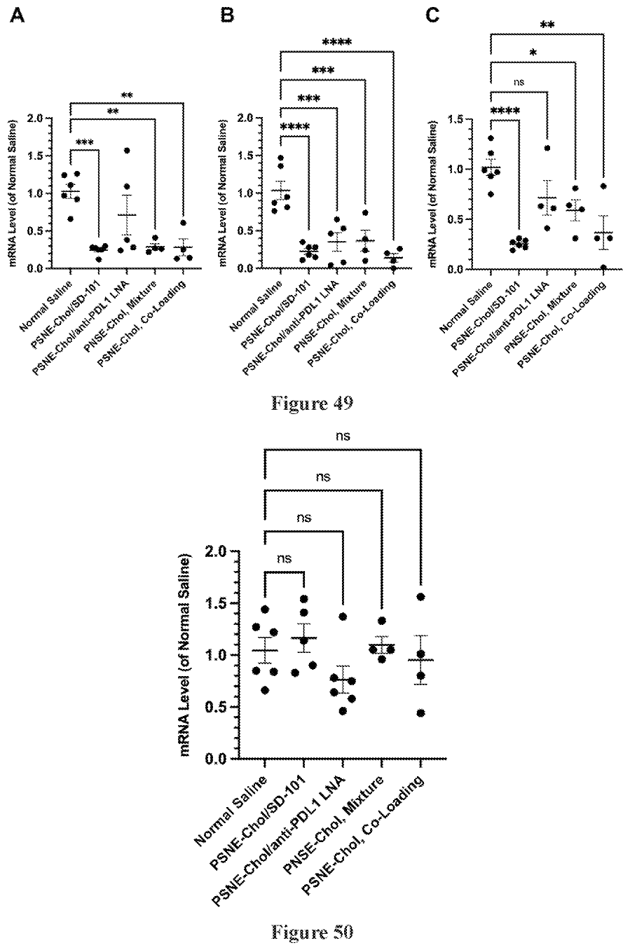

Next, splenocyte populations were examined to elucidate the effects of next-gen PSNE delivery platform on the immune cells. As shown in FIG. 47, both the population frequencies of CD4⁺ and CD8⁺ T lymphocytes in spleens were decreased after treatments in all treatment groups, matching the previous trends as well, but at further lower frequencies. It was interesting that the PSNE-Chol/M5-anti-PDL1 LNA ASO LNP single-agent group had broader distributions on both T lymphocyte populations. The decrease in both T lymphocyte populations was related to the dosing amount of the SD-101 CpG ODN, as all the treatments had the same lipid doses throughout the treatment period. In the meanwhile, the regulatory T lymphocyte (FIG. 48, CD4⁺ Foxp3⁺) populations were not significantly affected by the treatments, as the PSNE-Chol/M5-SD-101 LNP single-agent group had a trend in lowering the T_{reg} population after the treatment. Surprisingly, the co-loading combination group had a significant increased population after the treatment but not the mixture combination. Those mice were administered with the same weight amounts of oligonucleotides and lipids but in different loading formats, suggesting that the encapsulation methods could lead to different therapeutic outcomes. Furthermore, the genes of interest in splenocytes were examined by RT-qPCR. The results were shown in FIG. 49, demonstrating some interesting findings. First, the splenocyte Pdl1 expressions were significantly downregulated in groups treated with SD-101 CpG ODN, but not anti-PDL1 LNA ASO, thought the PSNE-Chol/M5-anti-PDL1 LNA ASO LNP still gave a decreasing trend in Pdl1 expression. Siglech and Foxp3 mRNA levels were examined as well, as those genes represented the signature cell populations plasmacytoid dendritic cells (pDC) and regulatory T lymphocytes, respectively. The RT-qPCR results demonstrated that all the treatment groups could significantly decrease the expression level of Siglech among the splenocytes, indicating the next-gen PSNE treatments could lead to a pDC population decrease, and the decrease might result from the re-distribution of pDC in spleens after DC activations or immune system activation. A similar trend was shown in Foxp3 expression as well, with the PSNE-Chol/M5-SD-101 LNP single-agent group having the least expression in Foxp3 mRNA, matching the result obtained from flow cytometry.

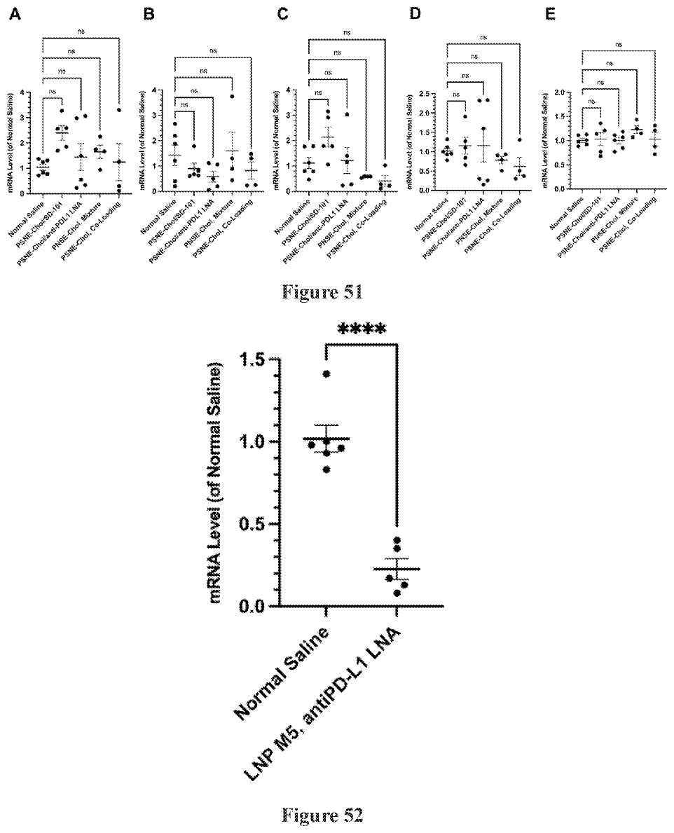

Not only the splenocyte mRNA expressions, but also the tumor single-cell mRNA expressions were examined as well. Tumor single-cell suspensions were obtained by dissociating the tumor tissues using proteases to remove the extracellular matrixes. The RT-qPCR results from tumor single-cell suspensions illustrated that there were no significant differences in Pdl1 expression, though PSNE-Chol/M5-anti-PDL1 LNA ASO LNP single-agent group showed lower Pd/J expression (FIG. 50). Furthermore, different cytokine mRNA expression levels were evaluated to elucidate the cytokine level within the tumor microenvironment indirectly. Although the results (FIG. 51) did not demonstrate any significant differences in Tnfa, Ifng, Il10, Il6, or Tgfb expressions, several key findings could be concluded from the results. Compared with the saline control group, the Tnfa expression was obviously increased in the PSNE-Chol/M5-SD-101 LNP single-agent group, suggesting that the secretion of the pro-inflammatory cytokine TNF-α, was higher in the tumor microenvironment, leading to the more cytotoxic environment.

Furthermore, based on the nature of nanoparticle biodistribution, anatomy of the liver, and the biodistribution assay utilizing DiR lipid near-infrared fluorescence dye-decorated PSNE-Chol/M5, the majority of lipid nanoparticles administered systemically accumulated in the liver with a minor (3% FL intensity of that at the liver, data not shown) in the spleen, livers of the PSNE-Chol/M5-anti-PDL1 LNA ASO LNP treatment group were analyzed for Pdl1 mRNA level using RT-qPCR. The RT-qPCR results (FIG. 52) indicated significant downregulation of hepatic Pdl1 mRNA, suggesting the majority of the PSNE-Chol/M5-anti-PDL1 LNA ASO LNP went to the liver and were uptaken by hepatocytes efficiently, leading to the mRNA degradation by LNA ASO and RNase H pathway.

### Discussion

In this Example, several strategies were tested to stimulate the immune system to fight against cancers. We selected the activation of dendritic cells through toll-like receptor activation as the central concept, and multiple strategies and combinations were tested utilizing different lipid nanoparticle formulations. In the early stage of the project, cationic nanoemulsions were used to encapsulate both hydrophobic agents, such as lipophilic TLR agonists or antigen peptides, and oligonucleotides into a single particle for delivery. In the late stage of the project, a newly developed pH-sensitive nanoemulsion lipid nanoparticle delivery platform was used to enhance the delivery of antisense oligonucleotides and CpG oligonucleotides.

To enhance the immune system activation, tumor associated antigens and toll-like receptor agonists are needed. In our first series of constructs, different TLR agonists were encapsulated into cationic nanoemulsions and were injected intratumorally. The antigen sources for dendritic cell activation were defined as the tumor cell debris from necrotic tumor cells, released proteins/fragments in exosomes or the phagocytosed dead cancer cells.

While the tumor microenvironment was full of useful antigens, the internal environment was anti-inflammatory, and the cytotoxic immune cell functions were inhibited by the released anti-inflammatory cytokines, such as TGF-β. Therefore, TLR agonists were needed to reactivate the immune cells, and the agonists were supplemented by nanoemulsions carrying imiquimod, CpG 2216, or poly(I:C). The preliminary study did not show significance in tumor growth inhibition, but some of the mice did have complete regression after the treatment, both in imiquimod (TLR7 agonist) and CpG 2216 (TLR9 agonist) treatment groups. Also, the tumor model selection was an important factor for evaluation, as Hepa1-6 syngeneic tumor model tend to generate larger ulcers and necrosis at the center of the tumor tissue, which was a good key factor for tumor associated antigen generation but would also lead to early removal as the illness and unhealthy ulcers could affect the overall animal health. The bleeding ulcers generated by Hepa1-6 model caused the early removal of mice in saline control group, generating bias against the treatment groups. Furthermore, mice in the treatment groups were removed due to the bleeding ulcer at the early stage of tumor progression, and that led to the bias in judging the therapeutic effect of the cancer vaccine. Overall, the preliminary experiment still gave us some useful information including the potency of different TLR agonists delivering intratumorally in vivo.

Next, we evaluated an alternative strategy in which defined tumor antigens along with TLR agonists or in combinations as a series of comprehensive cancer vaccine constructs were encapsulated. P1A peptide is a well-defined peptide antigen for murine J558 myeloma and has been reported to be successful to trigger protective response against J558 myeloma. Since P1A is a hydrophobic peptide, it can be successfully dissolved in squalene oil core as a solvate. Besides adding P1A to the nanoemulsion constructs, several TLR agonist combinations were tested, including MPLA (TLR4), Resiquimod (TLR7/8) and CpG 2216 or SD-101 (TLR9). MPLA and Resiquimod are lipophilic substances and can be dissolved in the squalene core as well. One the other hand, CpG 2216 and SD-101 are oligonucleotide-based cargos and have to be encapsulated into lipid nanoparticles through electrostatic interactions. The combination protective therapy, or the cancer vaccines, did show some promising results while co-loading three different TLR agonists together in a single particle instead of two or one of them. The result was interesting since the cancer vaccines were given subcutaneously and might have to boost the response as the general concept suggested to reactivate memory immune cells. The data suggested that a single shot of cancer vaccines might not be useful for generating anticancer immunity or other routes of administration might be needed.

Besides applying tumor associated antigens along with TLR agonists, we further considered delivering anti-PDL1 antisense oligonucleotides to downregulate PD-L 1 surface expressions on tumor cells to enhance the immune response as what was achieved by anti-PDL1 or anti-PD1 antibodies as immune checkpoint blockade therapies. The immune checkpoint signaling required the interaction between PD-L1 and PD-1 or PD-1 and CTLA-4 to inhibit the immune activation response on CD4⁺ and CD8⁺ T lymphocytes, which are the two of the main populations for anticancer immunity. Therefore, SD-101, the TLR9 agonist, and anti-PDL1 antisense oligonucleotides were encapsulated separately in standard lipid nanoparticles to enhance the delivery of oligonucleotides to cells and increase the circulation time. However, the overall outcome was not favorable for using anti-PDL1 ASO LNP as single therapy, while SD-101 LNP worked efficiently as a single agent. As the matter of fact, lipid nanoparticles were intended to deliver the cargos to liver, as liver is the major organ for foreign substance elimination. The sinusoids and capillaries in the liver could entrap the nanoparticles as the sizes were similar. The majority of the nanoparticles went to the liver, which was the nature of the lipid nanoparticle physiology. Further, we found that the modification on the ASO affected the efficacy significantly, as previously reported and as the in vitro evaluation done in-house by comparing 2′-OMe modified ASO with LNA modified ASO. The result also indicated that the failure of the anti-PDL1 ASO LNP single therapy could be resulted from the inefficient cargo carried in the lipid nanoparticles, as the standard lipid nanoparticle with DLin-MC3-DMA being a golden standard from delivery with high delivery efficiency.

To figure out a solution and develop a delivery platform for immune system activation therapy against cancer, a pH-sensitive nanoemulsion-based lipid nanoparticle delivery platform was developed to accommodate both hydrophobic substances and nucleic acid substances in a single construct. Several combinations were evaluated including TLR agonist combinations or combinations of TLR agonists with anti-PDL1 LNA ASO. The preliminary results presented in this Example were promising, with a high TGI ˜70%.

### Example 4: Squalene Emulsion Co-loaded Ivermectin and Resiquimod Promotes Systemic Antitumor Immunity

### Overview

Endosomal TLR agonists worked effectively as anticancer agents by facilitating antigen-presenting processes in dendritic cells (DCs) and augmenting CD8+ T lymphocyte (or cytotoxic T lymphocyte, CTL) maturation, which could rapidly recognize and kill cancer cells by T cell-mediated immunity. In the present study, a squalene-based nanoemulsion (NE) formulation was developed to co-deliver resiquimod (R848), a TLR7/8 agonist, and ivermectin (IVM), an anti-parasitic drug used worldwide since 1975. R848-IVM co-loaded NE was developed and characterized for stability. Antitumor activity of R848-IVM NE was also evaluated in vitro and in vivo. In vivo studies demonstrated that IVM could augment the immunogenic cell death induced by R848 and showed strong antitumor activity with over 80% tumor growth inhibition. Significant HMGB1 release into the tumor microenvironment was observed in mice treated with R848-IVM co-loaded NE. Over 3-fold increase in Cd8a expression was also observed in tumor tissues. The results suggested a potential combination therapy of systemic co-delivering IVM with other immune stimulation agents against solid cancer.

### Introduction

Toll-like receptors (TLRs) play critical roles in immune responses by recognizing pathogen-associated molecule patterns (PAMP) followed by inducing cytokine production and activating adaptive immunity. TLRs are expressed either on cellular surfaces (TLR1/2/4/5/6/10) or on endosomal surfaces (TLR3/7/8/9) of antigen-presenting cells (APCs) such as dendritic cells (DCs) or macrophages. TLRs are poised to recognize foreign molecular patterns, initiate MyD88/NF-κB transduction pathway, and activate naïve T cell repertoires in the adaptive immune systems. Studies have demonstrated that endosomal TLR agonists worked effectively as adjuvants in cancer vaccines due to their strong immunostimulatory abilities and antitumor efficacies. Endosomal TLR agonists have been shown to facilitate antigen-presenting processes in DCs and augment CD8+ T lymphocyte (or cytotoxic T lymphocytes, CTL) maturation, which could eventually suppress cancer cell growth through T cell-mediated immunity. The antitumor activity carried out by endosomal TLR agonists suggests that cell-mediated immunity derived from TLR activations could be beneficial for anticancer therapies while combining with personalized antigen-based therapies, immune checkpoint blockades, chemotherapeutics, or radiotherapies. Three TLR agonists have been approved by FDA for cancer treatments, including bacillus Calmette-Guerin (TLR2&4 agonists mixture), monophosphoryl lipid A (TLR2/4 agonist), and imiquimod (TLR7 agonist). Overall, the clinical outcomes of TLR agonists were mixed. Intra-tumoral or intradermal injection of TLR agonists has been investigated recently. However, these routes of administration are difficult to access during clinical practice for most solid tumors

During the treatment of TLR agonists against cancer, immunogenic cell death (ICD) plays an important role in antitumor immunity by rapidly inducing surface exposure of high-mobility group box 1 protein (HMGB1), calreticulin, as well as tumor antigens release to immune cells. Chronic exposure of these damage-associated molecular patterns (DAMPs) will recruit DCs and facilitate CTL activation through antigen presentation. The activated DCs and CTLs will accelerate the pace of engulfment of antigenic components in the tumor microenvironment (TME) and result in long-term antitumor immunity. Patients treated with traditional chemotherapies exhibited ICD-mediated antitumor immunity with an increased ratio of CTLs to regulatory T cells (Tregs) in their TMEs. The ICD-mediated antitumor response could also be augmented by immune checkpoint blockade which impedes immune escape between tumor cells and immune cells. However, chemotherapies are often associated with high cytotoxicity which causes dose-dependent damage to normal cells even if been administered locally to lower the systemic side effects. On the other hand, immune checkpoint blockade became a revolutionary approach to activate patients' immune systems to treat cancer. However, anticancer efficacy by immune checkpoint inhibitors is limited to “hot tumors” with abundant tumor-infiltrating immune cells, whereas “cold tumors” with limited immune cell infiltration showed minor responses to immune checkpoint inhibitors. Therefore, an immunomodulating therapy with enhanced systemic immune activation, rapid induction of ICD, and low toxicity would be an ideal approach for systemic anticancer treatment.

Resiquimod (R848) is a TLR7/8 agonist that has been shown to have ideal antitumor activities in murine tumor models. However, a single treatment of R848 was insufficient to induce systemic immune responses against tumors. Ivermectin (IVM) is an anti-parasitic drug used worldwide since 1975. Research has shown that IVM had the potential to alter the release of HMGB1 and calreticulin in TME, making it an ideal candidate to induce ICD in addition to R848 treatment. Nonetheless, due to the limited solubility of R848 and IVM, an injectable formulation is needed for clinical translation. Oil-in-water nanoemulsions (NE) have been proposed as promising non-viral delivery systems for hydrophobic drugs. NE consists of an oil core encapsulated by surfactants, where the oil core could work as an efficient reservoir to solubilize poor water-soluble drugs. Our work above demonstrated a squalene-based NE to encapsulate R848, a TLR7/8 agonist, which showed moderate antitumor activity through systemic administration.

In this Example, squalene-based NE was developed to co-encapsulate R848 and IVM. The squalene NE greatly increased the solubility of R848 and IVM in an aqueous solution, which turned the hydrophobic drugs feasible for systemic administration. The squalene-based NE eliminated the autophagy-associated cytotoxicity from IVM but maintained its ability to promote ICD. The R848-IVM co-loaded NE showed high stability when stored at 4° C. and −20° C. IVM NE treatment successfully induce HMGB1 releasement out from TMEs and increase Cd8a mRNA expression in tumor tissues. The antitumor efficacy of R848-IVM co-loaded NE (R848-IVM NE) was superior to R848 NE or IVM NE, suggesting the potential of combination therapies using TLR agonists along with IVM.

### Materials and Methods

### Materials.

Squalene was obtained from Sigma-Aldrich (St. Louis, MO, USA). 1,2-dioleoyl-sn-glycero-3-phosphocholine (DOPC) was purchased from Avanti Polar Lipids (Birmingham, AL, USA). Polysorbate 80 (Tween 80) was purchased from Fisher Scientific (Hampton, NH, USA). R848 and IVM were purchased from MedChemExpress (Monmouth Junction, NJ, USA) and Sigma-Aldrich, respectively. Any other reagents, including but not limited to buffers, were all obtained from Fisher Scientific.

### R848-IVM NE Formulation and Characterization.

Squalene-based NE was prepared by hand-rapid injection of oil-lipid mixture into phosphate-buffered saline (PBS). Squalene, DOPC, and Tween 80 were prepared at a molar ratio of 1/1/1 in ethanol. R848 and IVM were then added to the lipid-ethanol solution individually or in combination, maintaining lipid to R848/IVM at 10:1 (w/w). The final lipid concentration of the NE was 8 mg/mL, and the final drug concentrations were 0.8 mg/mL for both. Particle sizes were measured by dynamic light scattering (DLS) using a NICOMP NANO ZLS Z3000 (Entegris, Billerica, MA, USA). Empty NE was generated using the same procedures without R848 or IVM. Empty NE and R848-IVM NE were stored at 4° C. before characterization, and at −20° C. for long-term stability test. Sepharose CL-4B size exclusion chromatography was performed to examine the encapsulation efficiency of R848 or IVM within the squalene nanoemulsions. Drug concentrations were determined by UV-Vis spectrometry at 320 nm (R848) or 245 nm (IVM) using a NanoDrop 2000 spectrophotometer. The loading efficiency was determined by the equation below:

$$Encapsulation Efficiency \% = \frac{\sum UV absprbance of fractions 3 - 6 \times volume}{\sum UV absorbance of all fractions \times volume} \times 100 \%$$

In the saturated solubility study, R848 or IVM were dissolved in PBS or formulated in NE at 2 mg/ml and incubated at room temperature for 24 hours. Insoluble solid drugs were pelleted down by centrifugation. Supernatants were collected for concentration analysis. R848 concentrations were quantified by UV-Vis spectrometry at 320 nm using a NanoDrop 2000 spectrophotometer. IVM concentrations were quantified by high-performance liquid chromatography (HPLC) using an isocratic mobile phase composed of water/methanol/acetonitrile (8:36:56, v/v/v), an C18 column (Kromasil 150-C-18, 4.6×150 mm), and PDA detection at 230 nm.

### Cell Culture.

RAW 264.7 murine macrophage cell line and MC38 murine colorectal carcinoma cell line were kind gifts given by Dr. Peixuan Guo and Dr. Christopher Coss at The Ohio State University College of Pharmacy, respectively. RAW 264.7 and MC38 were grown in DMEM supplemented with 10% FBS and 1× antibiotic-antimycotic. Cells were maintained at 37° C. and grown under a humidified atmosphere containing 5% CO₂.

### Cell Viability Assay.

MC38 cells were seeded at 3000 cells/well in 96-well plates 24 hours before treatments. Cells were treated with R848, IVM, R848 NE, or IVM NE with escalated concentrations from 1 μM to 400 μM. A separate experiment was set up to evaluate the potential cytotoxicity of empty NE. Empty NE was treated with concentrations ranging from 10Ig/mL to 4000pg/mL. After 72 hour-treatment, cell viability was examined by CellTiter 96R AQueous One Solution (Promega, Madison, WI) per manufacturer protocol. The IC50 for R848 or IVM was determined by R programming.

### In Vitro Gene Regulation by R848 NE, IVM NE, and R848-IVM NE.

MC38 cells were seeded at 3×10⁵ cells/well in 6-well plates 24 hours before treatments. Cells were treated with R848, IVM, R848 NE, IVM NE, or R848-IVM NE in complete media and incubated for 24 hours. R848 and IVM were both treated at 8 μM, either as free drug solution or in squalene NE. Total RNA was extracted using TRI reagent (Zymo Research) per manufacturer protocol. cDNA was prepared by high-capacity cDNA reverse transcription kit (Invitrogen, Waltham, MA, USA), and real-time qPCR (RT-qPCR) was done using SsoAdvanced™ Universal SYBRR Green Supermix (Bio-Rad Laboratories, Hercules, CA) on a QuantStudio 7 Flex Real-time PCR System. RT-qPCR primers for murine Calreticulin, Hmgb1, Lc3b, and Actb were purchased from Sigma-Aldrich. Actb was selected as the housekeeping gene control. The relative amount of RNA level was calculated and compared according to the 2−ΔΔCt method.

### In Vitro Tumor Cell Migration Assay.

A scratch wound healing model was conducted to examine the migratory ability of MC38 cells following treatment. MC38 cells were seeded at a density of 5×10⁵ cells/well in 6-well plates 24 hours before treatments. A scratch wound across the well was made using a 10 ul pipet tip immediately before treatment. Cells were washed by PBS and incubated with complete media containing 8 μM of R848, IVM, R848 NE, IVM NE, or R848-IVM NE. Cells were allowed to proliferate at 37° C. for 24 hours. Distances between edges of the wound were measured by Nikon Eclipse Ti-S microscope (Nikon, Tokyo, Japan).

### In Vivo Antitumor Activity.

MC38 murine syngeneic colorectal cancer model was generated by inoculating 1×10⁶ cells/mouse in PBS on the right flanks of C57BL/6N mice (Charles River Laboratories). Treatments were initiated once tumor sizes reached approximately 100 mm3. Mice (n=5) were intraperitoneally treated with saline, 4 mg/kg R848 NE, 4 mg/kg IVM NE, or R848-IVM NE (4 mg/kg R848 and 4 mg/kg IVM) every 3 days for 3 doses. Tumor growth and body weight were monitored, and the tumor volumes were calculated according to the equation below:

$$Tumor Volume = \frac{Length \times {Width}^{2}}{2}$$

All mice were maintained and treated according to the guidelines from the Institutional Animal Care and Use Committee (IACUC) at The Ohio State University. All groups were euthanized on day 9, and whole-blood samples were collected through cardiac puncture. Mouse sera were collected by placing whole-blood samples at room temperature for 30 minutes followed by 2000×g centrifugation for 20 minutes at RT. Samples were stored at −80° C. before cytokine quantification. Murine TNF-α and IL-6 concentrations were determined by TNF-α and IL-6 mouse uncoated ELISA kits (Invitrogen, Waltham, MA, USA) per manufacturer protocol. Tumor and spleen tissues were harvested and weighed for comparison. Spleen weight was normalized to individual bodyweight for comparison between treatment groups. Tumor growth inhibition (% TGI) on day 10 was determined by the equation below:

$$\% TGI = \frac{1 - ( \frac{{T}_{10}}{{T}_{0}} / \frac{{C}_{10}}{{C}_{0}} )}{1 - \frac{{C}_{10}}{{C}_{0}}} \times 100 \%$$

where T₁₀ stands for average tumor volume of the treatment group at day 10, To stands for average tumor volume of the treatment group at day 0, Cio stands for average tumor volume of the control group at day 10, and Co stands for average tumor volume of the control group at day 0. % TGI>50% was considered meaningful.

### In Vivo Gene Regulation by R848 NE, IVM NE, and R848-IVM NE.

Tumor and spleen tissues were immediately harvested at the end of treatment. Spleens were homogenized into single-cell suspensions by pressing through 70 μm cell strainers with syringe plungers. Splenocytes were washed and resuspended in FACS staining buffer at 2×10⁷ cells/mL. 1×10⁶ cells of splenocytes were saved for flow cytometry analysis. The remaining splenocytes were lysed in TRI reagent for total RNA extraction. Tumor tissues were homogenized directly in TRI reagent through probe sonication. Total RNAs were isolated per manufacturer protocol. RT-qPCR was completed according to the procedures stated in section 2.5. The sequences of real-time qPCR assay primers for murine Cd3e, Cd4, and Cd8a were listed below.

| Target | | | GenBank |
|---|---|---|---|
| Gene | Forward Sequence | Reverse Sequence | Number |
| Cd3e | GCTCCAGGATTTCTCGGA | ATGGCTACTGCTGTCAGG | NM_007648.5 |
| | AGTC (SEQ ID: 4) | TCCA (SEQ ID: 5) | |
| Cd4 | GTTCAGGACAGCGACTTC | GAAGGAGAACTCCGCTG | NM_013488.3 |
| | TGGA (SEQ ID: 6) | ACTCT (SEQ ID: 7) | |
| Cd8a | ACTACCAAGCCAGTGCTG | ATCACAGGCGAAGTCCAA | NM_001081110.2 |
| | CGAA (SEQ ID: 8) | TCCG (SEQ ID: 9) | |

### In Vivo Protein Expression Analysis by Western Blot.

Tumors were harvested and homogenized in Pierce RIPA buffer (Thermo Fisher Scientific) using a handheld homogenizer. Total proteins were extracted after incubating on ice for 30 minutes and centrifuged at 14000×g for 30 minutes at 4° C. Protein samples were denatured, quantified, and equal amounts of proteins were loaded and electrophoresed on 10% SDS-polyacrylamide gels and transferred onto nitrocellulose membranes. The transferred membranes were blocked with 5% nonfat milk in Tris-buffered saline (TBS). HMGB1 rabbit monoclonal antibody and anti-rabbit HRP conjugated secondary antibody were purchased from Cell Signaling (Danvers, MA, USA). Ki-67 rabbit monoclonal antibody was purchased from Thermo Fisher Scientific. Specific target protein bands were developed with enhanced chemiluminescence (ECL) detection system and total proteins were detected by Ponceau S as control.

### Flow Cytometry.

APC/Cyanine7 anti-mouse CD3e (145-2C11), FITC anti-mouse CD4 (RM4-5), PE/Cyanine7 anti-mouse CD8a (53-6.7), PE anti-mouse FOXP3 (MF-14), and True-Nuclear Transcription Factor Buffer Set for FOXP3 staining were purchased from Bio-Legend (San Diego, CA, USA). Single-cell suspensions of splenocytes in FACS staining buffer were stained per manufacturer protocol. Stained cells were analyzed using an LSR II flow cytometer in Flow Cytometry Shared Resources (FCSR) at The Ohio State University Comprehensive Cancer Center.

### Statistical Analysis.

All studies were done in triplicate. Data are presented as means±standard deviations unless otherwise indicated. Statistical analysis will be conducted using Microsoft Excel. One-way ANOVA was used to determine variances in means between two or more treatment groups. Student's t test was used as a post-hoc analysis to determine statistically significant differences between any two groups. A p-value of 0.05 was selected as the cutoff for statistical significance.

### Results and Discussion

### Particle Characterization and Solubility.

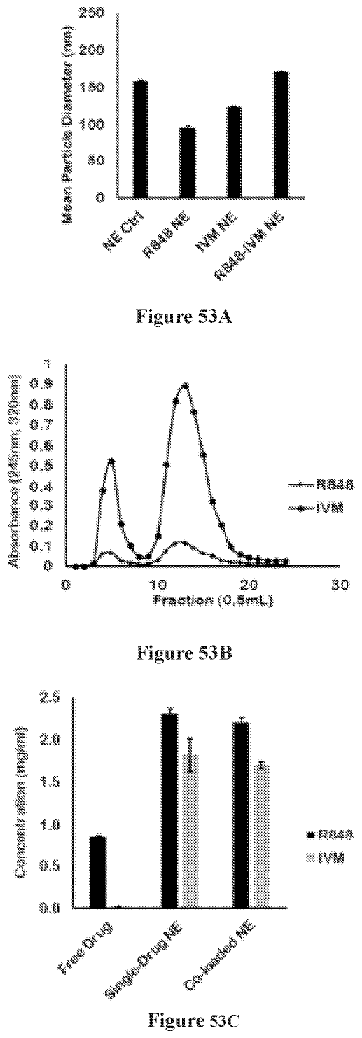

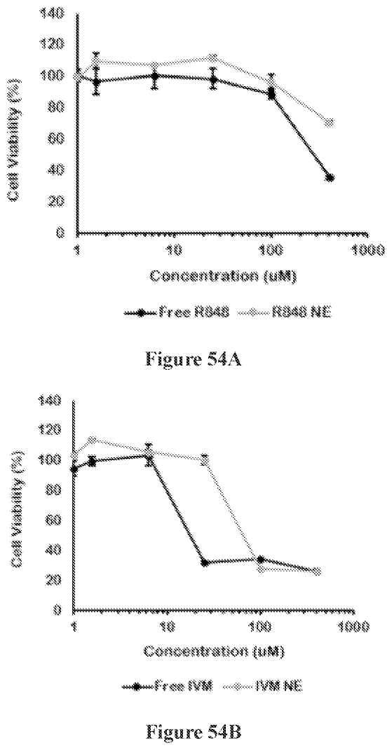

IVM is a broad-spectrum antiparasitic agent against many endo- and ecto-para-sites. Recent studies suggested that IVM exhibited certain antitumor activities through multiple pathways in many types of tumors. However, severe side effects have been reported when high doses of IVM were administered. Previous research has introduced different lipid-based nanoparticles to deliver IVM against parasites. However, a systemic delivery platform for IVM against cancers has not been well established. In our previous Examples, a squalene-based NE was capable to encapsulate R848 and achieved great potential of eliminating tumor growth through systemic administration in tumor-bearing mice when combined with other TLR agonists. Herein, squalene-based NE was capable of co-encapsulating IVM and R848. R848-IVM NE was approximately 140-160 nm in size,
which was larger than IVM NE or R848 NE (FIG. 53A). Nonetheless, the particle size of R848-IVM NE is considered suitable for cellular uptake based on published studies. R848-IVM NE also exhibited high colloidal stability under 4° C. and −20° C. storage for over 6-month with encapsulation efficiency (%) 21.77±2.10 (STD) for R848 232 and 22.80±0.13 (STD) for IVM. The squalene-based NE successfully increased the solubility of R848 by 2-fold and IVM by 100-fold compared with the solubility in PBS solvent (FIG. 53C) which provides an efficient systemic delivery platform and could expand the indications for both R848 and IVM in clinics.

### In Vitro Cell Viability.

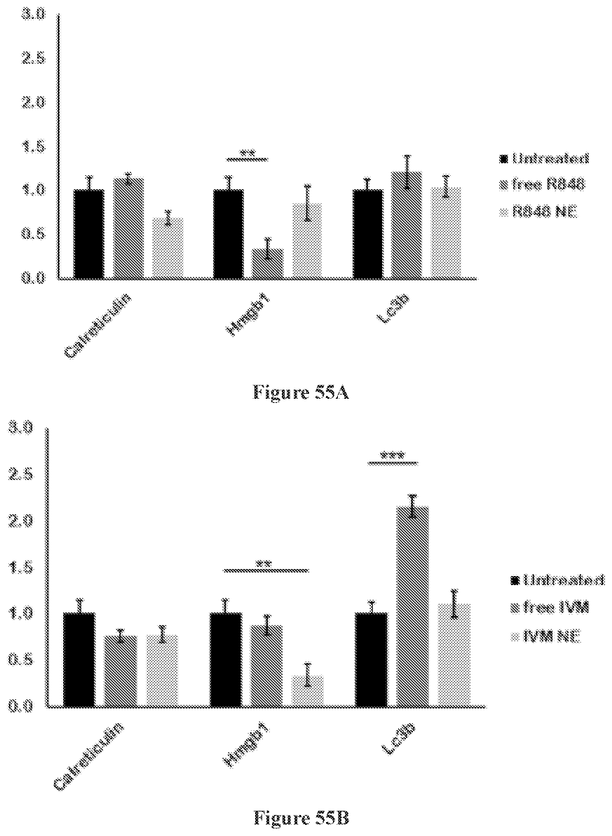

Although most TLR7/8 agonists, such as R848, have no direct cytotoxic effects in vitro or in vivo, many reports have shown that IVM confers significant toxicity through autophagy and DNA damages. Treatments with empty NE in MC38 cells showed no significant cytotoxicity at the highest dose of 4000pg/ml. Likewise, treatments with free R848 and R848 NE did not result in significant cytotoxicity (IC50>100 μM), analyzed by MTS assay (FIG. 54A). However, IVM NE exhibited a higher IC50 of 43.52+23.53 μM (SEM) compared with free IVM of IC5o at 10.94±4.15 μM (SEM) (FIG. 54B), suggesting that squalene-based NE successfully reduced the cytotoxicity carried out by the IVM.

### In Vitro Gene Regulation by Free Drugs and NE Formulation.

The process of immunogenic cell death in tumors is mainly mediated by DAMPs released from cancer cells, which includes surface exposure of calreticulin, releasement of HMGB1 and type I interferons (IFNs). DAMPs are further recognized by APCs and CTLs to induce antitumor immunity. Many studies have shown that calreticulin translocation and exposure were two important components as ICD checkpoints, and knockdown of calreticulin completely abolished the immunogenicity in tumors during the ICD process. In addition, HMGB1 has been shown to facilitate ICD from the extracellular compartment including triggering tumor necrosis factor-alpha (TNFa) releasement, APC maturation, and CTLs recruitment. However, endogenous caireticulin and HMGB1 exhibit controversial roles in cancer progression. Endogenous HMGB1 was demonstrated to promote cancer cell proliferation and CRT has pro-angiogenic functions due to its ability to promote the expression of vascular endothelial growth factor (VEGF) which leads to cancer cell proliferation and migration as well.

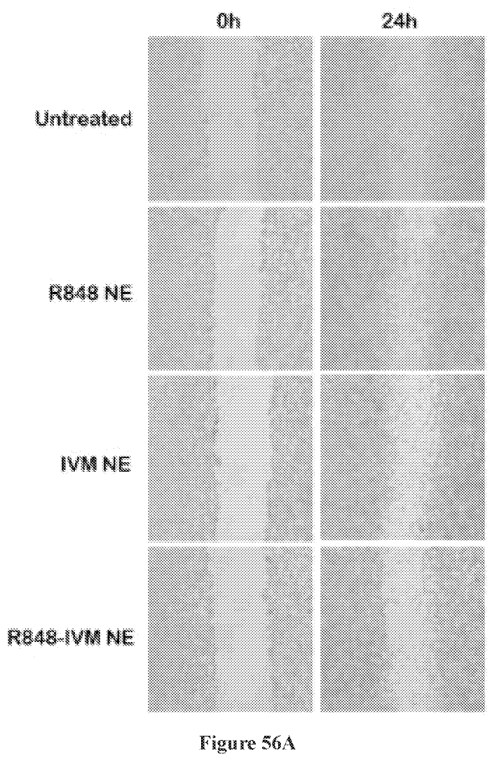

In MC38 cells, Calreticulin mRNA level was slightly decreased after R848 NE treatment, indicating the potential of cell growth inhibition (FIG. 55A). No significant Hmgb1 mRNA regulation was observed in R848 NE treatment, though free R848 showed Hmgb1 downregulation (FIG. 55A). IVM and IVM NE did not show significant Calreticulin regulation at the mRNA level, whereas IVM NE significantly downregulates Hmgb1 (FIG. 55B), suggesting a new antitumor pathway of IVM NE by inhibiting cell proliferation.

IVM has been demonstrated to trigger autophagy-mediated cell death by blocking PAK1/Akt axis and generating LC3-II in autophagosomes, where LC3 is a key protein participating in initiating autophagy. In the present study, squalene-based NE mitigated the autophagy-mediated cell death from IVM, which can be identified by reduced Lc3b mRNA expression to normal level in IVM NE treatment compared with free IVM treatment (FIG. 55B). The result suggested that IVM NE would be suitable for systemic administration with low cytotoxicity which corresponded with the cell viability results of free IVM and IVM NE (FIG. 54B).

### Wound Healing.

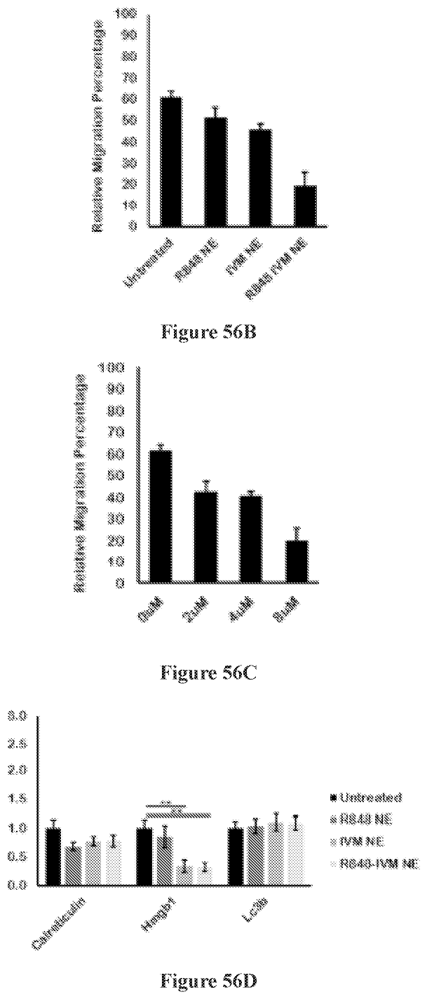

Wound healing studies were performed to monitor the relative mobility of MC38 following treatment with R848 NE, IVM NE, or R848-IVM NE (FIG. 56A). R848 NE did not confer a significant decrease in cell migration in the wound region since R848 does not possess any inhibitory effects on cell growth but only immunostimulatory properties. The cell mobility was reduced to 37.57%±8.33% with IVM NE treatment and to 19.37%±4.89% with R848-IVM NE treatment (FIG. 56B). The decreased cell mobility can be due to significant Hmgb1 downregulation in IM NE and R848-IVM NE treatments (FIG. 56D), which correlates with the established role of HMGB1 in promoting cell proliferation. The lower mobility in R848-IVM NE may be attributed to the addictive cell growth inhibition through calreticulin (no statistical significance) and Hmgb1 mRNA downregulation by R848 and IVM in NE. The R848-IVM NE also exhibited concentration-dependent mobility inhibition of 43.85%, 40.32%, and 19.37% treated at 2 μM, 4 μM, and 8 μM, respectively (FIG. 56C).

### In Vivo Antitumor Activity.

In the animal study to evaluate the antitumor efficacy for R848-IVM NE, 4 mg/kg R848 NE and IVM NE were administered individually or as R848-IVM NE to show a combined antitumor efficacy through intraperitoneal administration. Moderate antitumor efficacy by R848 NE treatment was observed compared with saline control and was consistent with our previous results. Studies have shown that IVM potentiated ICD processes and promoted antitumor immunity for immune checkpoint inhibitors. However, no significant antitumor effect has been observed in mice treated with IVM NE compared with saline control, which corresponds with previous research on the IVM (FIG. 57, Panel C, Panel F-Panel H). Nonetheless, a strong antitumor effect has been observed in mice treated with R848-IVM NE compared with R848 NE and saline control (FIG. 57, Panel F-Panel H).

R848-IVM NE has reached 88.66%±14.91% in tumor growth inhibition (TGI %) at the end of the study, which was superior to R848 NE and IVM NE treatments (Table 4). No significant weight loss was observed during the treatment regimen, suggesting minor systemic toxicity of R848-IVM NE treatment. Mice treated with R848 NE or R848-IVM NE exhibited slight splenomegaly. The increase in spleen weights was caused by the immune activation carried out by R848. No significant changes in spleen weight were observed in mice treated with IVM NE. No significant changes in serum cytokine level were observed.

TABLE 4
Tumor growth inhibition (TGI %) at day 10
for R848 NE, IVM NE, and R848-IVM NE.

| Treatment | Mean | Standard |
|---|---|---|
| Group | TGI % | Deviation |
| R848 NE | 64.33 | 10.75 |
| IVM NE | 43.27 | 22.12 |
| R848-IVM NE | 88.66 | 14.91 |

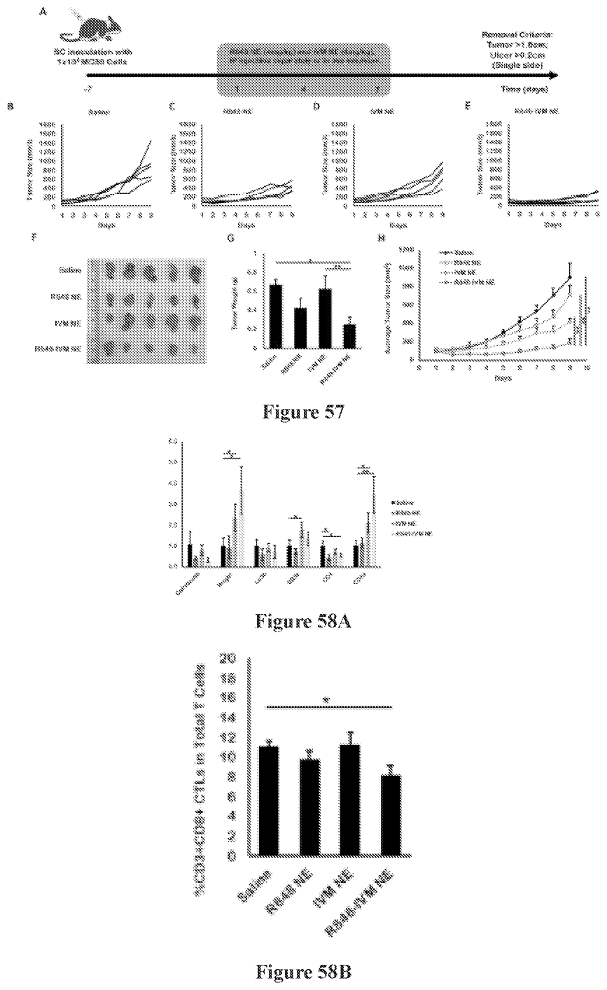

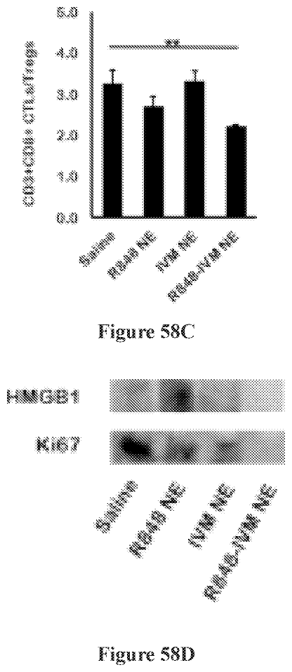

Effects of R848 and IVM on Gene Regulation and Immune Cell Population in vivo. Although no significant cytokine increase in serum was observed in mice treated 359 with R848 NE, IVM NE, or R848-IVM NE, RT-qPCR results of tumor tissues suggested that IVM NE and R848-IVM NE significantly induce significant Hmgb1 mRNA upregulation (FIG. 58A). Meanwhile, western blot results showed low detection of HMGB1 in tumor tissues collected from mice treated with IVM NE and R848-IVM NE, indicating the ICD process that Hmgb1 mRNA was overexpressed within TME but HMGB1 protein was released into the extracellular compartment, which corresponds with previous studies on the regulation of HMGB1 by IVM treatment. Significant Calreticulin mRNA downregulation and low Ki67 protein level were observed in tumor tissues from mice treated with R848-IVM NE (FIGS. 58A and 58D), suggesting a proliferation-unfavorable TME was generated by R848-IVM NE treatment.

Flow cytometry data indicated that CTL populations in mouse spleens were reduced after treatment with R848 NE and R848-IVM NE (FIGS. 58B and 58C), which did not correlate with the immune activation effects in splenomegaly carried out by R848. However, a significant increase in Cd8a mRNA level was observed in tumor tissues collected from mice treated R848-IVM NE (FIG. 58A), which potentially suggested that R848-IVM NE treatment could successfully induce CTLs and/or NK cells infiltration into TME from spleens. The upregulation of Cd8a mRNA would also be attributed to IVM, which was shown in the tumor tissues from IVM NE single treatment group (FIG. 58A).

### Conclusion

Although ICD-mediated antitumor immunity has been demonstrated to facilitate the antitumor response by both traditional chemotherapies and immune checkpoint blockades in many cancer patients, chemotherapies are often correlated with unwanted side effects and toxicity, which causes dose-dependent damage to normal cells even being administered locally to lower drug concentrations within systemic circulation. On the other hand, the anticancer efficacy by immune checkpoint inhibitors is limited to tumors that have already been infiltrated by T cells, whereas tumors with less T cell infiltration showed minor responses to immune checkpoint inhibitors. The present study showed a promising strategy to achieve systemic antitumor activity through augmenting the process of ICD by using combination therapy of R848 and IVM co-encapsulated squalene-based NE. R848-IVM NE is highly stable during long-term storage at 4° C. and −20° C. The squalene-based NE greatly reduced the cytotoxicity carried out by IVM. Ultimately, R848-IVM NE strongly enhanced the antitumor activity in vivo by greatly suppressing tumor growth over 80%. The results indicated that intraperitoneal administration of R848-IVM NE could be a promising strategy to induce ICD and recruit CTLs and NK cells to TME, suggesting a broader application of IVM in inducing ICD while combining with TLR agonists or other immunotherapeutic against cancer.

### Example 5: pH Sensitive Nanoemulsions (PSNEs) for the Delivery of Nucleic Acid Cargos

PNSEs comprise similar components as conventional LNPs (e.g., ionizable lipid, neutral lipid, cholesterol, and PEG-lipid). In some embodiments, the PNSEs can further include a fusogenic oil such as squalene or squalene to promote membrane fusion and endosomal release of nucleic acid (NA) drugs. The addition of the fusogenic oil can increase the efficiency of intracellular delivery of the PSNE cargo.

PSNE or LNP-based delivery of NA is heavily influenced by the surface properties of the particles. Because the lipid particles include an ionizable lipid (e.g., DLinMC3DMA, SM-102, or ALC-0315), the pH of the composition (and the pKa of the ionizable lipid) can significantly influence delivery efficiency. However, previously reported formulations all have a final pH that is either neutral or slightly basic. For example, Patisiran (LNP with siRNA) has a pH of ˜7; both Moderna and Pfizer covid vaccines have a pH of (7-8, or 7.5). At these pH ranges, the ionizable lipid is mostly uncharged, resulting in a very low zeta potential for the particles. This in turn results in minimum cellular uptake since electrostatic interaction between the particles and the negatively charged cell surface is negligible, and low delivery efficiency in vitro and in vivo at physiological extracellular pH (7.4).

In some embodiments, the PSNEs can be buffered at an acidic pH (e.g., a pH of less than 6.5, such as a pH of from 4 to 6.5, or a pH of from 5 to 6.5), which results in a dramatic increase in cellular delivery efficiency both in vitro and in vivo.

### Materials and Methods

Squalene was purchased from Sigma-Aldrich (St. Louis, MO). DOPC and DOPE were purchased from Avanti Polar Lipids (Alabaster, AL). DODMA and DMG-PEG2000 were purchased from NOF America (White Plains, NY). DLinDMA and DLin-MC3-DMA were purchased from MedChemExpress (Monmouth Junction, NJ). Any chemicals or buffers otherwise stated were purchased from Fisher Scientific (Hampton, NH).

DOPC, DOPE, squalene, ionizable lipids (including, but not limited to DODMA and DLin-MC3-DMA) and DMG-PEG2000 were mixed into a lipid ethanol mixture at a molar ratio of 15:28:10:45:2. Next, the lipid ethanol mixture was rapidly injected into an acidic phosphate buffer to form empty pH-sensitive nanoemulsion. In the meanwhile, nucleic acid cargos, including but not limited to messenger RNA (mRNA), small interference RNA (siRNA) or antisense oligonucleotides (ASO), were dissolved in DEPC water at desired concentrations. Empty pH-sensitive nanoemulsions and nucleic acid cargos were heated up to 60° C. prior to the mixing. Then, the cargo was added dropwise to the empty pH-sensitive nanoemulsion on slow vortex at a weight ratio of 1 to 15-25 until nucleic acid-encapsulated lipid nanoparticles were formed. The products were incubated at 37° C. for 10 minutes and stored in 4° C. before use. The particle sizes and zeta potential (ζ) of nucleic acid-loaded pH-sensitive nanoemulsion were analyzed by dynamic light scattering with a NICOMP Z3000 Nano DLS/ZLS Systems (Entegris, Billerica, MA).

Cells were maintained in either Dulbecco's Modified Eagle Medium (DMEM) or Minimum Essential Media (MEM) supplemented with L-glutamine (2 mM), 10% FBS, sodium pyruvate (1 mM) and antibiotics-antimycotic (Thermo Fisher Scientific, Pittsburgh, PA) at 37° C. with 5% C02. Cells were homogenized in TRI reagent, and total RNA was extracted per manufacturer's protocol. cDNA was prepared using High-Capacity cDNA Reverse Transcription Kit per manufacturer's protocol. Real-time PCR was conducted on a QuantStudio 7 Flex Real-Time PCR System. The relative amount of RNA level was calculated and compared according to the 2^{-ΔΔCt} method.

C57BL/6 and ICR mice were purchased from Charles River Laboratory. Animals were housed in a temperature-controlled room under a 12 hr light/12 hr dark cycle and under helicobacter-free conditions and fed normal chow diet. All animal studies were reviewed and approved by the Ohio State University Institutional Laboratory Animal Care and Use Committee. Both male and female mice were used for experiments.

In vivo mRNA expression was quantified by measuring firefly luciferase bioluminescence. Mice treated with IM injection of FFLuc mRNA lipid nanoparticles were injected with luciferin intraperitoneally 5 minutes prior to bioluminescence measurements. Bioluminescence images were taken by a in vivo imaging system (IVIS) under optimal exposure settings.

### Results

### Evaluation of Squalene as a Fusogenic Oil.

To compare the fusogenicities of different oils, simple anionic nanoemulsions were prepared. The compositions evaluated were: oil/DOPC/CHEMS (30:40:40 mole/mole). Oil was selected from squalene, medium-chain triglyceride (MCT) from Abitec (Captex 300), long-chain triglyceride (LCT, soybean oil from Sigma-Aldrich). DOPC: dioleoylphosphatidylcholine, CHEMS: cholesterol hemisuccinate. The various formulations were prepared at pH 8.0 in PBS and then diluted into various buffers with different pH values and incubated for 5 min at room temperature prior to OD measurement. OD500 and OD600 were measured to indicate particle size, with larger particle size indicating increased fusogenicity.

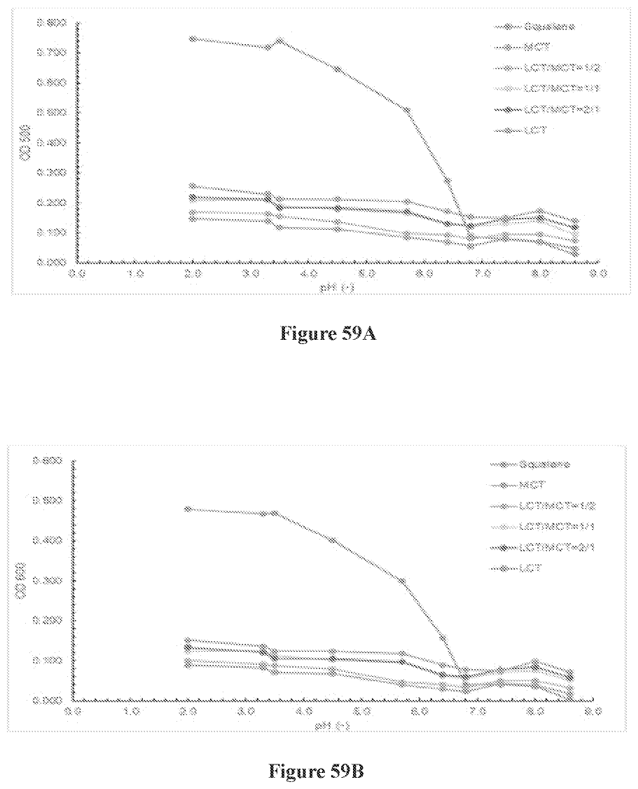

The results are shown in FIGS. 59A and 59B. As shown in FIGS. 59A and 59B, squalene exhibited improved fusogenicity as opposed to other representative oils (medium-chain triglyceride and/or long-chain triglyceride).

In Vitro Activity of PSNEs Loaded with siRNA Against Akt-1 in KB Cells.

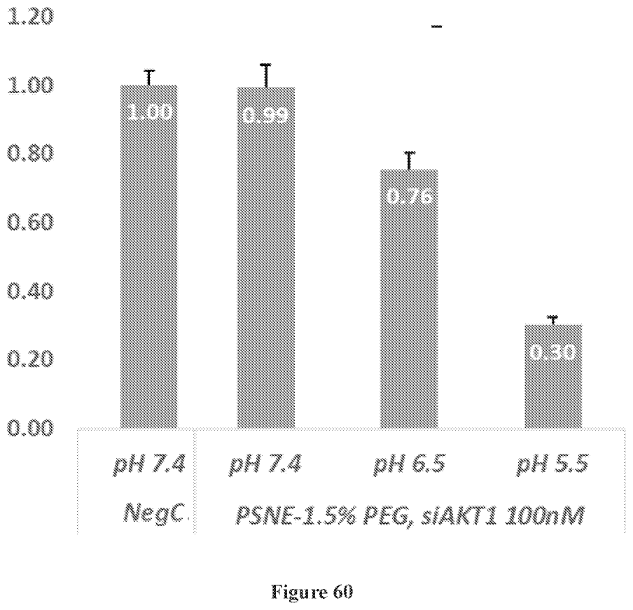

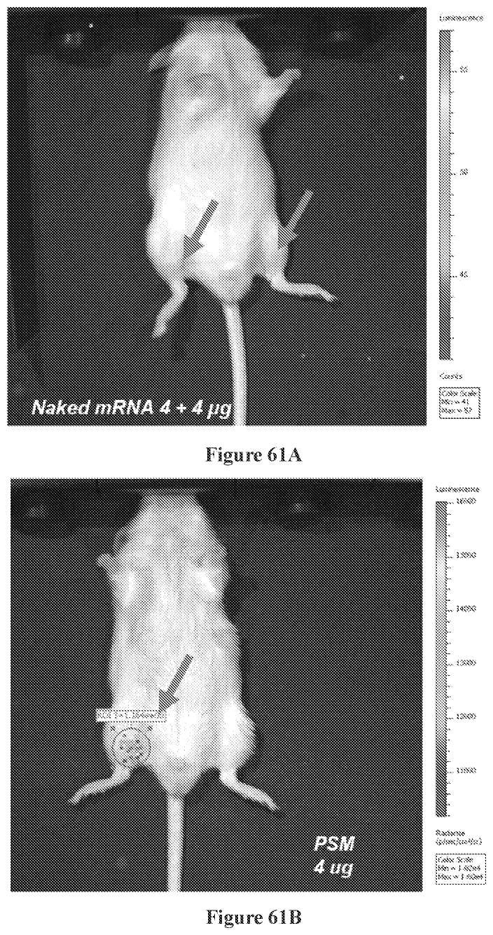

In this proof-of-principle study, a PSNE formulation loaded with siRNA showed much greater target gene Akt-1 downregulation when cellular incubation was performed at acidic pH values (5.5 and 6.5) compared to when the incubation was performed at the typical media pH of 7.4 (see FIG. 60). The results have since been confirmed using various siRNA and mRNA cargos and are highly reproducible.

### Evaluation of PSNE Nucleic Acid Delivery In Vivo.

The efficacy of PSNE formulations for delivery in vivo were evaluated using luciferase mRNA reporter gene-loaded PSNE formulations. PSNEs loaded with FFLuc mRNA were buffered at pH 6 were administered to mice by intramuscular injection. For comparison, mice were similarly treated with FFLuc mRNA alone (naked mRNA), FFLuc mRNA encapsulated in LNPs, FFLuc mRNA encapsulated in pH-sensitive micelles (PSMs).

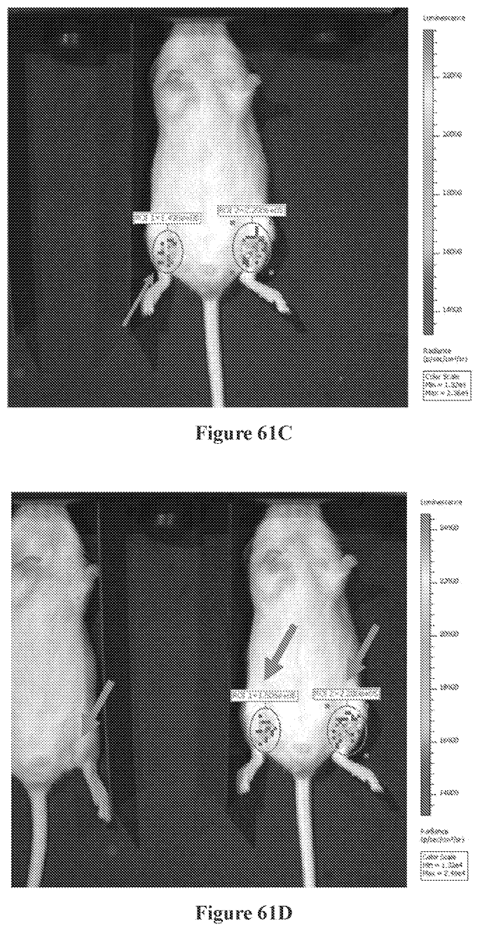

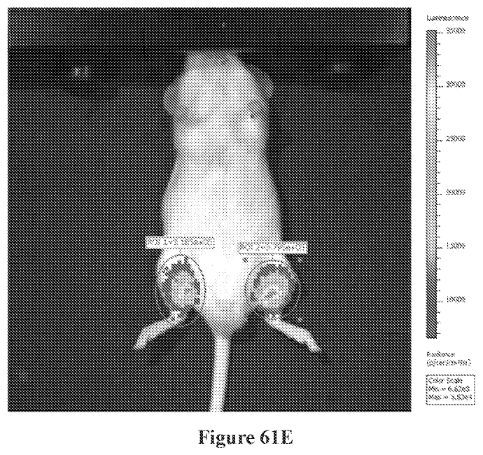

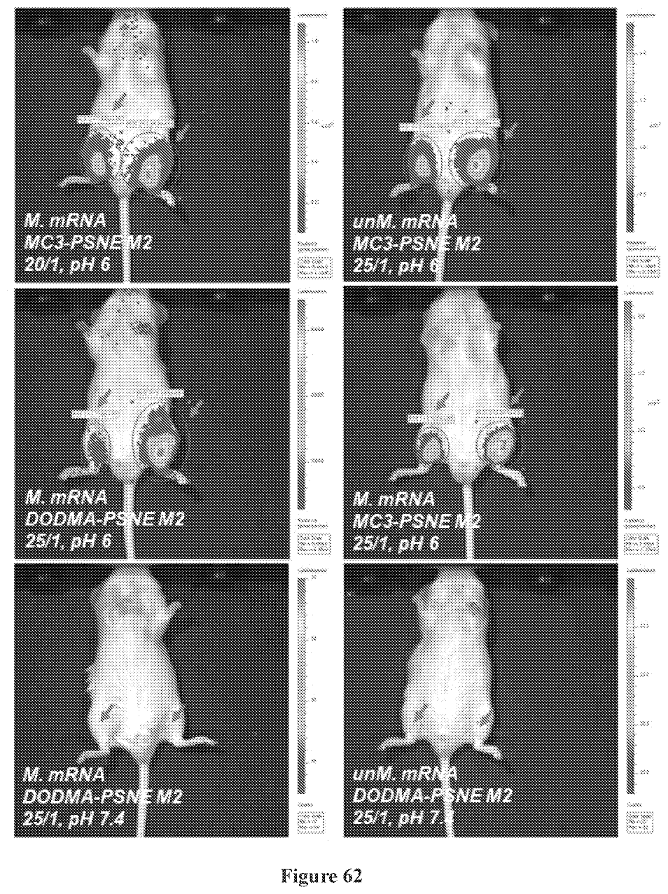

FIGS. 61A-61E show in vivo bioluminescence imaging of the mice treated with i.m. injection of FFLuc mRNA (naked mRNA), FFLuc mRNA encapsulated in LNPs, FFLuc mRNA encapsulated in pH-sensitive micelles (PSMs), and FFLuc mRNA encapsulated in PSNEs (buffered at pH 6). As shown in FIGS. 61A-61E, the PSM and PSNE formulations were far more active than the LNP formulation, with the PSNE formulation exhibiting the highest activity in mRNA delivery.

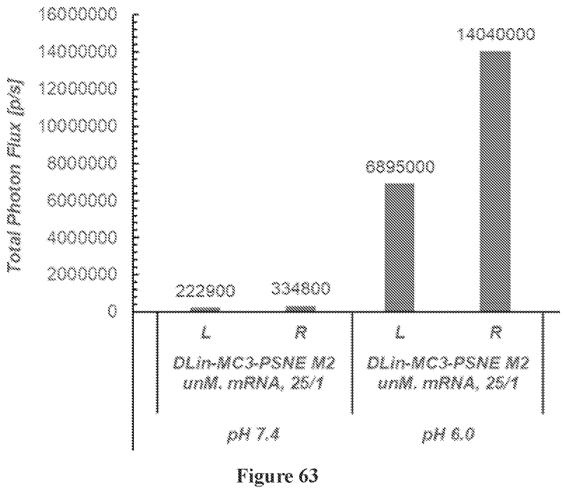

FIG. 62 shows the delivery afforded by PSNEs based on Dlin-MC3-DMA and based on DODMA carrying luciferase mRNA given to mice by i.m. injection. A high level of gene expression was seen for PSNEs in 50 mM pH 6 histidine buffer. However, when the formulations were adjusted to pH 7.4 using phosphate buffer, in vivo gene expression was no longer detectable. This result is quantified in FIG. 63, which includes a plot of the increase in bioluminesnce observed following i.m. injection of PSNEs based on Dlin-MC3-DMA carrying luciferase mRNA buffered in 50 mM pH 6 histidine buffer versus 50 mM phosphate buffer pH 7.4. A 30-50× enhancement in luminsence was observed by lowering the pH from 7.4 to 6. These results suggest a very strong pH dependence for the formulation for i.m. delivery of mRNA.

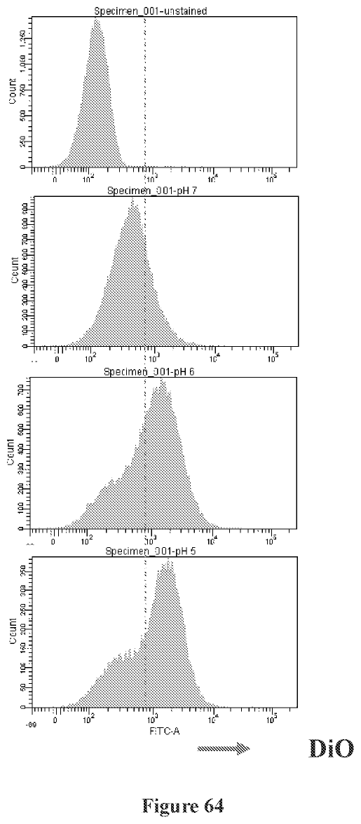

Similar results were observed in ex vivo studies. As shown in FIG. 64, the cellular uptake of fluorescence-labeled PSNEs in KB cells at differing buffering pH showed much higher levels of cellular uptake (increased mean fluorescence intensity) by flow cytometry analysis as pH 5 and pH 6 as compared to pH 7. This illustrates a mechanism of pH-dependent cellular uptake/delivery of PSNEs.

There are no previous examples of LNP formulations with an acidic final pH for in vitro or in vivo applications. All previously reported formulations have a neutral or slightly basic pH for the final formulation that roughly matches the pH of physiological value (7.4).

### Discussion

Without wishing to be bound by theory, it is believed that inducing a positive zeta potential on lipid nanoparticles will increase cellular uptake due to electrostatic interaction with the negatively charged cell surface. In the case of lipid-based delivery vehicles that include ionizable lipids, this positive charge is only present at acidic pH. By buffering such compositions at acidic pH values, the compositions can exhibit improved cellular uptake.

In vivo, a low pH formulation of PSNE or LNP (therefore having a positive surface charge) can similarly interact with the target cell (e.g., during i.m. injection). This can result in greatly increased i.m. delivery efficiency compared to a formulation in a buffer with pH of 7-8. This is demonstrated by the luciferase mRNA reporter gene and i.m. injection into mice in a pH 6 histidine buffer, showing vastly superior gene expression compared to the same formulation in pH 7 phosphate buffer. Alternatively, the particles can become coated by plasma proteins (e.g., during i.v. injection) to a greater extent at a lower pH than at a pH of 7-8 (when the particles are mostly uncharged). The protein coating rapidly achieved based on cationic surface charge will result in a different biodistribution pattern, and the adsorbed proteins can facilitate target cell uptake (e.g., binding of acidic albumin (pI 4.7) can facilitate albumin receptor-mediated endocytosis).

There are several important consequences of LNP or PSNE formulations in an acidic final buffer. First, the optimal pKa of the ionizable lipid may now significantly deviate from the previous reports. A pKa of 6.2-6.5 may no longer be the optimal pKa due to the change in buffering pH, so commonly available ionizable lipids such as DODMA can now show much higher activity compared to their use in previous studies (when formulations were in pH 7-8).

The practical applications include the following:
(1) Previously it has been difficult to show NA delivery in vitro using formulations containing ionizable lipid, making comparisons between ionizable lipids difficult due to low activity. With the acidic pH strategy, it is now possible to produce highly efficient delivery in vitro, even in difficult to transfect cells. This has potential application in ex vivo transfection protocols, using ionizable lipids which are less cytotoxic than permanently charged lipids. Ex vivo NA delivery does have application in AAV production plasmid transfection, CAR-T and gene editing. This concept of acidic pH transfection can be extended to ionizable polymers such as poly-histidine and polyethylenimine by enhancing their in vitro gene transfer activity for ex-vivo applications.
(2) For i.m. and other local injection routes (intrathecal, intracerebral, intravitreal etc.), acidic buffer (e.g., 50 mM histidine pH 6) can maintain local pH and promote PSNE or LNP delivery of NA (e.g., mRNA) into the local tissue (e.g., muscle fiber). Clinical applications include mRNA vaccines for viruses and cancer (e.g., COVID vaccines).
(3) When LNP or PSNE in acidic buffer is given i.v., pH is equilibrated to the plasma pH, however, before that happens, the particles would have been coated with negatively charged plasma protein such as albumin and this facilitate cellular uptake in the liver or in the tumor, and results in increased tissue uptake and cellular delivery efficiency.

In summary, lipid nanoparticles including an ionizable lipid component can be formulated in an acidic buffer to improve delivery efficiency. In addition, we have also demonstrated that a fusogenic oil (e.g., squalene) can be incorporated into lipid nanoparticles including an ionizable lipid to improve delivery efficiency. These concepts can be employed independently or in combination. Applications include formulations for the in vitro, ex vivo, in vivo local injection, and in vivo systemic administration of active agents such as (but not limited to) nucleic acids.

### Example 6: Device for Rapid Synthesis of Ethanol-Free Lipid Nanoparticles

Lipid nanoparticles are typically synthesized by mixing lipids dissolved in ethanol and nucleic acid in an aqueous buffer at a 1:3 volume ratio. However, this process generates nanoparticles containing 25% ethanol. Removal of ethanol is typically accomplished by dialysis at a small scale or diafiltration at a large scale, which is a time-consuming process that is clearly beyond the capability of hospital pharmacies thus precludes bedside or hospital pharmacy-based manufacturing.

In this example, we integrated a cartridge of size-exclusion resin in a syringe-pump system for ethanol removal. As a result, ethanol-free and buffer-exchanged nanoparticles are generated in a single step in real-time. This has never been accomplished before and greatly simplified the nanoparticle synthesis process, enabling nanoparticle generation at the bedside using kit formulations or pre-filled syringe assemblies. This will be a decisive advantage for personalized cancer vaccine applications where nanoparticle synthesis is required at a small scale and individualized. Nanoparticle manufacturing can be accomplished by the push of a button in a hospital pharmacy setting by a technician with limited training. This is crucial in the implementation of individualized cancer vaccine production. Tumor antigens coded by different mRNAs can be produced in a large variety and individualized nanoparticles generated using the selected mRNA.

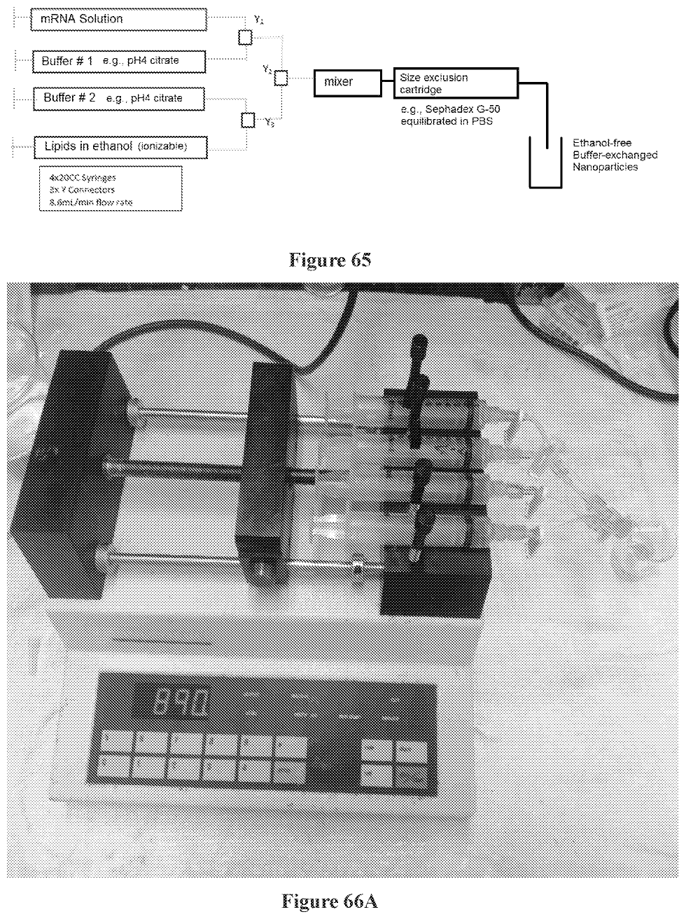

FIG. 65 shows the design of a syringe assembly with integrated size-exclusion cartridge for in-line ethanol removal and buffer exchange. Nanoparticle formation and ethanol removal are accomplished by a push of a single button using a pre-assembled single-use syringe-cartridge assembly. The syringes can be pre-filled or loaded on-site from kit-based vials labeled for each syringe (a total of 4 vials per kit). This system was able to generate nanoparticles with a high degree of reproducibility. The Mixer can be a simple narrowing of the fluid path via a double-hub needle or an inline micromixer. The pump pushes the solutions through the mixer element and then through the size exclusion cartridge resulting in ethanol-free nanoparticles by limiting the total volume to less than 75% of the volume of the total volume of the cartridge. Ethanol is trapped in the dead volume of the assembly and the resin in the cartridge. For example, an 8-mL cartridge enabled the production of 6 mL of ethanol-free nanoparticle product.

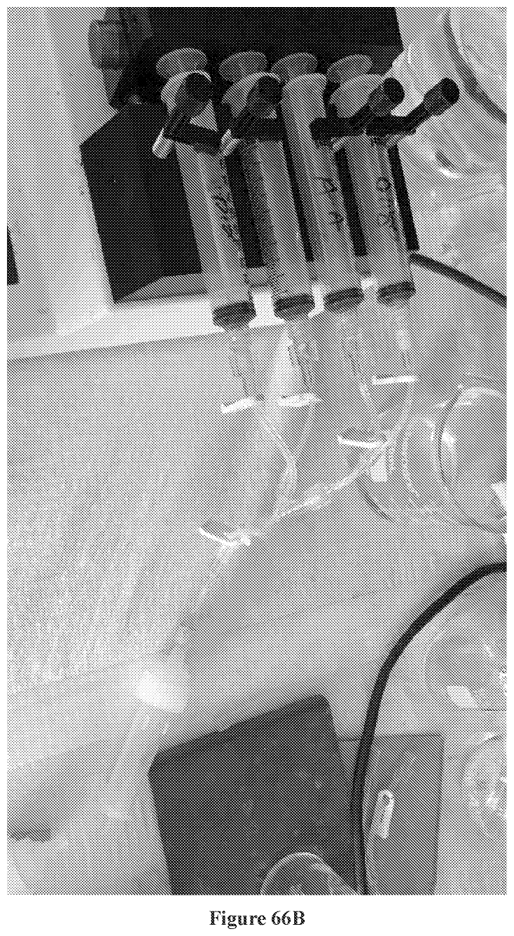

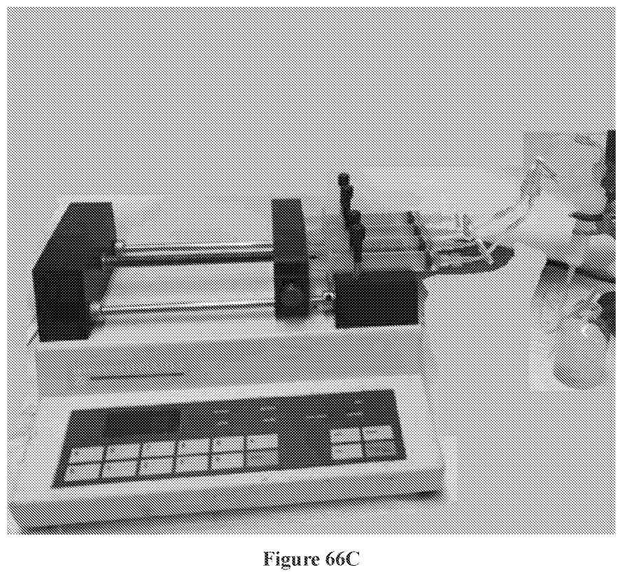

FIGS. 66A-66C are photographs showing a 4-syringe+1-cartridge assembly for instant generation of ethanol-free nanoparticles. The cartridge shown was a Sorbent Technologies Flash Cartridge with 8-mL volume. In a typical run, the cartridge is packed with Sephadex G-50 resin preequilibrated in PBS. The syringes are loaded with 1.5-ml each of the mRNA, lipid, or buffer solutions. The pump is started by the start button and 6 mL of ethanol-free and buffer exchanged nanoparticles (in PBS) is generated in a receiving vial instantly. The particle size obtained was 100-200 nm by dynamic light scattering. The yield (% recovery of nucleic acid compared to input) for producing ethanol-free nanoparticles with this device was 68.8%.

So the “injection device” is basically a pump-driven device that mixes ethanolic lipid solution with an aqueous nucleic acid solution for instant self-assembly of nanoparticles (via nanoprecipitation). The specific configuration enabling 1:3 mixing ratio and the pump setup is unique, convenient to operate, and compatible with standard syringe pump drivers and standard syringe sets. The integration with in-line cartridge for instant ethanol removal is also a unique feature. Although recovery nucleic acid is limited to ˜50%, the product is ethanol-free. Instant ethanol removal has not been achieved previously and is critical for individualized nanoparticle manufacturing. This device is equally efficacious for producing microparticles or microspheres instantly. This device can be used to make individualized cancer vaccines by making the mRNA reagent interchangeable while maintaining the remainder of the single-use assembly. In addition, ethanol can be removed instantly without requiring dialysis or diafiltration, making on-site nanoparticle preparation feasible.

The compositions and methods of the appended claims are not limited in scope by the specific compostions and methods described herein, which are intended as illustrations of a few aspects of the claims. Any compositions and methods that are functionally equivalent are intended to fall within the scope of the claims. Various modifications of the compositions and methods in addition to those shown and described herein are intended to fall within the scope of the appended claims. Further, while only certain representative compounds, components, compositions, and method steps disclosed herein are specifically described, other combinations of the compounds, components, compositions, and method steps also are intended to fall within the scope of the appended claims, even if not specifically recited. Thus, a combination of steps, elements, components, or constituents may be explicitly mentioned herein or less, however, other combinations of steps, elements, components, and constituents are included, even though not explicitly stated.

The term “comprising” and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments of the invention and are also disclosed. Other than where noted, all numbers expressing geometries, dimensions, and so forth used in the specification and claims are to be understood at the very least, and not as an attempt to limit the application of the doctrine of equivalents to the scope of the claims, to be construed in light of the number of significant digits and ordinary rounding approaches.

Unless defined otherwise, all technical and scientific terms used herein have the same meanings as commonly understood by one of skill in the art to which the disclosed invention belongs. Publications cited herein and the materials for which they are cited are specifically incorporated by reference.

SEQ ID NO: 1
mrifavfifm tywhllnaft vtvpkdlyvv eygsnmtiec kfpvekqldl aalivyweme dkniiqfvhg
eedlkvqhss yrqrarllkd qlslgnaalq itdvklpqdag vyrcmisygg adykritvkv napynkinqr
ilvvdpvtse heltcqaegy pkaeviwtss dhqvlsgktt ttnskreekl fnvtstlrin tttneifyct
frrldpeenh taelvipelp lahppnerth lvilgaillc lgvaltfifr lrkgrmmdvk kcgiqdtnsk
kqsdthleet
SEQ ID NO: 2
cgaggctccg caccagccgc gcttctgtcc gcctgcaggg cattccagaa agatgaggat
atttgctgtc tttatattca tgacctactg gcatttgctg aacgcattta ctgtcacggt
tcccaaggac ctatatgtgg tagagtatgg tagcaatatg acaattgaat gcaaattccc
agtagaaaaa caattagacc tggctgcact aattgtctat tgggaaatgg aggataagaa
cattattcaa tttgtgcatg gagaggaaga cctgaaggtt cagcatagta gctacagaca
gagggcccgg ctgttgaagg accagctctc cctgggaaat gctgcacttc agatcacaga
tgtgaaattg caggatgcag gggtgtaccg ctgcatgatc agctatggtg gtgccgacta
caagcgaatt actgtgaaag tcaatgcccc atacaacaaa atcaaccaaa gaattttggt
tgtggatcca gtcacctctg aacatgaact gacatgtcag gctgagggct accccaaggc
cgaagtcatc tggacaagca gtgaccatca agtcctgagt ggtaagacca ccaccaccaa
ttccaagaga gaggagaagc ttttcaatgt gaccagcaca ctgagaatca acacaacaac
taatgagatt ttctactgca cttttaggag attagatcct gaggaaaacc atacagctga
attggtcatc ccagaactac ctctggcaca tcctccaaat gaaaggactc acttggtaat
tctgggagcc atcttattat gccttggtgt agcactgaca ttcatcttcc gtttaagaaa
agggagaatg atggatgtga aaaaatgtgg catccaagat acaaactcaa agaagcaaag
tgatacacat ttggaggaga cgtaatccag cattggaact tctgatcttc aagcagggat
tctcaacctg tggtttaggg gttcatcggg gctgagcgtg acaagaggaa ggaatgggcc
cgtgggatgc aggcaatgtg ggacttaaaa ggcccaagca ctgaaaatgg aacctggcga
aagcagagga ggagaatgaa gaaagatgga gtcaaacagg gagcctggag ggagaccttg
atactttcaa atgcctgagg ggctcatcga cgcctgtgac agggagaaag gatacttctg
aacaaggagc ctccaagcaa atcatccatt gctcatccta ggaagacggg ttgagaatcc
ctaatttgag ggtcagttcc tgcagaagtg ccctttgcct ccactcaatg cctcaatttg
ttttctgcat gactgagagt ctcagtgttg gaacgggaca gtatttatgt atgagttttt
cctatttatt ttgagtctgt gaggtcttct tgtcatgtga gtgtggttgt gaatgatttc
ttttgaagat atattgtagt agatgttaca attttgtcgc caaactaaac ttgctgctta
atgatttgct cacatctagt aaaacatgga gtatttgtaa aaaaaaaaaa aaa

What is claimed is:

1. A pharmaceutical composition comprising a lipid particle encapsulating an active agent, the lipid particle comprising:
one or more ionizable lipids;
one or more neutral lipids; and
one or more PEGylated lipids;
wherein the composition is buffered at a pH of from 5.0 to 6.5.

2. The composition of claim 1, one or more ionizable lipids are present in the lipid particle in an amount of from 20 mol % to 65 mol % of the total components forming the lipid particle.

3. The composition of any of claims 1-2, one or more neutral lipids are present in the lipid particle in an amount of from 35 mol % to 80 mol % of the total components forming the lipid particle.

4. The composition of any of claims 1-3, one or more PEGylated lipids are present in the lipid particle in an amount of from greater than 0 mol % to 5 mol % of the total components forming the lipid particle.

5. A pharmaceutical composition comprising a lipid particle encapsulating an active agent, the lipid particle comprising:
from 20 mol % to 65 mol % one or more ionizable lipids;
from 35 mol % to 80 mol % one or more neutral lipids;
from greater than 0 mol % to 5 mol % one or more PEGylated lipids; and
from 5 mol % to 50 mol % one or more fusogenic oils.

6. The composition of claim 5, wherein the composition is buffered at an acidic pH.

7. The composition of claim 6, wherein the acidic pH is from 5.0 to 6.5.

8. The composition of any of claims 5-7, wherein the one or more fusogenic oils are present in the lipid particle in an amount of from 10 mol % to 40 mol % of the total components forming the lipid particle.

9. The composition of any of claims 5-8, the fusagenic oil comprises a C12-C40 hydrocarbon comprising fewer than 3 rings.

10. The composition of claim 9, wherein the C12-C40 hydrocarbon comprises an alkyl or alkylene chain.

11. The composition of claim 10, wherein the C12-C40 hydrocarbon comprises an alkylene chain optionally comprises a least one cis-double bond.

12. The composition of any of claims 5-11, wherein the fusogenic oil comprises squalene, squalane, pristane, pristene, farnesene, farnesane, retinol, phytol, a carotene, a tocopherol, a tocotrienol, phytomenadione, menaquinone, where valence permits esters thereof, and combinations thereof.

13. The composition of any of claims 5-12, wherein the fusogenic oil comprises squalene.

14. The composition of any of claims 1-13, wherein the one or more ionizable lipids are present in the lipid particle in an amount of from 30 mol % to 50 mol % of the total components forming the lipid particle.

15. The composition of any of claims 1-14, wherein the one or more ionizable lipids comprise a lipid headgroup comprising a tertiary amine.

16. The composition of any of claims 1-15, wherein the one or more ionizable lipids comprise N,N-dimethyl-2,3-dioleyloxypropylamine (DODMA), [(4-hydroxybutyl)azanediyl]di(hexane-6,1-diyl)bis(2-hexyldecanoate) (ALC-0315); 9-heptadecanyl 8-{(2-hydroxyethyl)[6-oxo-6-(undecyloxy)hexyl]amino}octanoate (SM-102), DLin-MC3-DMA; DLin-KC2-DMA; or any combination thereof.

17. The composition of any of claims 5-16, wherein the fusogenic oil and the one or more ionizable lipids are present in the lipid particles at a molar ratio of from 0.25:1 to 1:1.

18. The composition of any of claims 1-17, wherein the one or more neutral lipids are present in the lipid particle in an amount of from 30 mol % to 50 mol % of the total components forming the lipid particle.

19. The composition of any of claims 1-18, wherein the one or more neutral lipids comprise dipalmitoylphosphatidylcholine (DPPC), dioleoylphosphatidylethanolamine (DOPE), palmitoyloleoylphosphatidylcholine (POPC), egg phosphatidylcholine (EPC), distearoylphosphatidylcholine (DSPC), cholesterol, or any combination thereof.

20. The composition of any of claims 1-19, wherein the one or more PEGylated lipids are present in the lipid particle in an amount of from greater than 0 mol % to 10 mol % of the total components forming the lipid particle.

21. The composition of any of claims 1-20, wherein the one or more PEGylated lipids comprise a PEG-ditetradecylacetamide, a PEG-myristoyl diglyceride, a PEG-diacylglycerol, a PEG dialkyloxypropyl, a PEG-phospholipid, a PEG-ceramide, or any combinations thereof.

22. The composition of any of claims 5-21, wherein the fusogenic oil and the one or more PEGylated lipids are present in the lipid particles at a molar ratio of from 5:1 to 20:1.

23. The composition of any of claims 1-22, wherein the lipid particles have an average diameter of less than 1 micron, such as from 50 nm to 750 nm, 50 nm to 250 nm, from 50 nm to 200 nm, from 50 nm to 150 nm, or from 50 nm to 100 nm.

24. The composition of any of claims 1-23, wherein the lipid particles have a polydispersity index (PDI) of less than 0.4.

25. The composition of any of claims 1-24, wherein the active agent comprises a nucleic acid.

26. The composition of claim 25, wherein the nucleic acid comprises siRNA, mRNA, or any combination thereof.

27. A method of delivering an active agent to a cell, the method comprising contacting the cell with the composition of any of claims 1-26.

28. A method for in vivo delivery of an active agent to a cell, said method comprising administering to a mammalian subject the composition of any of claims 1-26.

29. The method of claim 28, wherein the mammal is a human.

30. The method of any of claims 28-29, wherein the administration is intravenous.
